SEQUENCE LISTING (166 sequences)

SEQ ID NO: 1 - RNA, 22 nt, Homo sapiens
uggagugugacaaugguguuug

SEQ ID NO: 2 - RNA, 23 nt, Homo sapiens
uuaaugcuaaucgugauaggggu

SEQ ID NO: 3 - RNA, 22 nt, Homo sapiens
cugugcgugugacagcggcuga

SEQ ID NO: 4 - RNA, 22 nt, Homo sapiens
uggcagugucuuagcugguugu

SEQ ID NO: 5 - RNA, 22 nt, Homo sapiens
uagcagcacauaaugguuugug

SEQ ID NO: 6 - RNA, 22 nt, Homo sapiens
ugucaguuugucaaauacccca

SEQ ID NO: 7 - RNA, 22 nt, Homo sapiens
uagcuuaucagacugauguuga

SEQ ID NO: 8 - RNA, 22 nt, Homo sapiens
aaaccguuaccauuacugaguu

SEQ ID NO: 9 - RNA, 22 nt, Homo sapiens
uagcagcacaucaugguuuaca

SEQ ID NO: 10 - RNA, 23 nt, Homo sapiens
ucucacacagaaaucgcacccgu

SEQ ID NO: 11 - RNA, 21 nt, Homo sapiens
caagucacuagugguuccguu

SEQ ID NO: 12 - RNA, 22 nt, Homo sapiens
uccuguacugagcugccccgag

SEQ ID NO: 13 - RNA, 21 nt, Homo sapiens
uaaagugcugacagugcagau

SEQ ID NO: 14 - RNA, 22 nt, Homo sapiens
ucucccaacccuuguaccagug

SEQ ID NO: 15 - RNA, 21 nt, Homo sapiens
aggggugcuaucugugauuga

SEQ ID NO: 16 - RNA, 22 nt, Homo sapiens
ugagaacugaauuccauaggcu

SEQ ID NO: 17 - RNA, 22 nt, Homo sapiens
cagugcaaugaugaaagggcau

SEQ ID NO: 18 - RNA, 23 nt, Homo sapiens
cuggagauauggaagagcugugu

SEQ ID NO: 19 - RNA, 22 nt, Homo sapiens
gugacaucacauauacggcagc

SEQ ID NO: 20 - RNA, 22 nt, Homo sapiens
ugagaacugaauuccauggguu

SEQ ID NO: 21 - RNA, 22 nt, Homo sapiens
uagcagcacguaaauauuggcg

SEQ ID NO: 22 - RNA, 22 nt, Homo sapiens
cacuagauugugagcuccugga

SEQ ID NO: 23 - RNA, 22 nt, Homo sapiens
uggagagaaaggcaguuccuga

SEQ ID NO: 24 - RNA, 22 nt, Homo sapiens
aaggagcucacagucuauugag

SEQ ID NO: 25 - RNA, 23 nt, Homo sapiens
caaagugcuguucgugcagguag

SEQ ID NO: 26 - RNA, 22 nt, Homo sapiens
uucaccaccuucuccacccagc

SEQ ID NO: 27 - RNA, 23 nt, Homo sapiens
aacauucauuguugucggugggu

SEQ ID NO: 28 - RNA, 23 nt, Homo sapiens
uagugcaauauugcuuauagggu

SEQ ID NO: 29 - RNA, 21 nt, Homo sapiens
gcaguccaugggcauauacac

SEQ ID NO: 30 - RNA, 21 nt, Homo sapiens
uggguuuacguugggagaacu

SEQ ID NO: 31 - RNA, 22 nt, Homo sapiens
uuauaaagcaaugagacugauu

SEQ ID NO: 32 - RNA, 19 nt, Homo sapiens
aaaagcuggguugagagga

SEQ ID NO: 33 - RNA, 22 nt, Homo sapiens
cauugcacuugucucggucuga

SEQ ID NO: 34 - RNA, 21 nt, Homo sapiens
cauuauuacuuuugguacgcg

SEQ ID NO: 35 - RNA, 22 nt, Homo sapiens
ugagguaggagguuguauaguu

SEQ ID NO: 36 - RNA, 22 nt, Homo sapiens
ugagguaguagauuguauaguu

SEQ ID NO: 37 - RNA, 22 nt, Homo sapiens
ugagguaguaguuugugcuguu

SEQ ID NO: 38 - RNA, 22 nt, Homo sapiens
ucguaccgugaguaauaaugcg

SEQ ID NO: 39 - RNA, 22 nt, Homo sapiens
ugagguaguagguuguauaguu

SEQ ID NO: 40 - RNA, 22 nt, Homo sapiens
ggauuccuggaaauacuguucu

SEQ ID NO: 41 - RNA, 22 nt, Homo sapiens
agagguaguagguugcauaguu

SEQ ID NO: 42 - RNA, 23 nt, Homo sapiens
uaaagugcuuauagugcagguag

SEQ ID NO: 43 - RNA, 22 nt, Homo sapiens
cacgcucaugcacacacccaca

SEQ ID NO: 44 - RNA, 22 nt, Homo sapiens
aaaagcuggguugagagggcaa

SEQ ID NO: 45 - RNA, 22 nt, Homo sapiens
ugagguaguagguuguaugguu

SEQ ID NO: 46 - RNA, 20 nt, Homo sapiens
aaaagcuggguugagagggu

SEQ ID NO: 47 - RNA, 22 nt, Homo sapiens
gggagccaggaaguauugaugu

SEQ ID NO: 48 - RNA, 22 nt, Homo sapiens
ugccuacugagcugaaacacag

SEQ ID NO: 49 - RNA, 21 nt, Homo sapiens
ugagaugaagcacuguagcuc

SEQ ID NO: 50 - RNA, 21 nt, Homo sapiens
ucgaggagcucacagucuagu

SEQ ID NO: 51 - RNA, 22 nt, Homo sapiens
cucccacugcuucacuugacua

SEQ ID NO: 52 - RNA, 22 nt, Homo sapiens
aaaagcuggguugagagggcga

SEQ ID NO: 53 - RNA, 23 nt, Homo sapiens
ugaggggcagagagcgagacuuu

SEQ ID NO: 54 - RNA, 22 nt, Homo sapiens
ugagguaguagguugugugguu

SEQ ID NO: 55 - RNA, 23 nt, Homo sapiens
ugagugugugugugugagugugu

SEQ ID NO: 56 - RNA, 23 nt, Homo sapiens
uagcagcgggaacaguucugcag

SEQ ID NO: 57 - RNA, 23 nt, Homo sapiens
caaagugcuuacagugcagguag

SEQ ID NO: 58 - RNA, 23 nt, Homo sapiens
aacauucaacgcugucggugagu

SEQ ID NO: 59 - RNA, 21 nt, Homo sapiens
uagcagcacagaaauauuggc

SEQ ID NO: 60 - RNA, 21 nt, Homo sapiens
cuagacugaagcuccuugagg

SEQ ID NO: 61 - RNA, 22 nt, Homo sapiens
uaugugccuuuggacuacaucg

SEQ ID NO: 62 - RNA, 20 nt, Homo sapiens
acugccccaggugcugcugg

SEQ ID NO: 63 - RNA, 23 nt, Homo sapiens
aaaagugcuuacagugcagguag

SEQ ID NO: 64 - RNA, 22 nt, Homo sapiens
ucaggcucaguccccucccgau

SEQ ID NO: 65 - RNA, 21 nt, Homo sapiens
cggggcagcucaguacaggau

SEQ ID NO: 66 - RNA, 21 nt, Homo sapiens
cagcagcacacugugguuugu

SEQ ID NO: 67 - RNA, 22 nt, Homo sapiens
uccauuacacuacccugccucu

SEQ ID NO: 68 - RNA, 23 nt, Homo sapiens
ucccccaggugugauucugauuu

SEQ ID NO: 69 - RNA, 23 nt, Homo sapiens
aaugacacgaucacucccguuga

SEQ ID NO: 70 - RNA, 22 nt, Homo sapiens
caacaaaucacagucugccaua

SEQ ID NO: 71 - RNA, 22 nt, Homo sapiens
ugagguaguaguuuguacaguu

SEQ ID NO: 72 - RNA, 22 nt, Homo sapiens
uauugcacucgucccggccucc

SEQ ID NO: 73 - RNA, 24 nt, Homo sapiens
uugcagcugccugggagugacuuc

SEQ ID NO: 74 - RNA, 22 nt, Homo sapiens
ucugcccccuccgcugcugcca

SEQ ID NO: 75 - RNA, 23 nt, Homo sapiens
uaaggugcaucuagugcagauag

SEQ ID NO: 76 - RNA, 21 nt, Homo sapiens
uucaaguaauucaggauaggu

SEQ ID NO: 77 - RNA, 22 nt, Homo sapiens
ccaauauuacugugcugcuuua

SEQ ID NO: 78 - RNA, 22 nt, Homo sapiens
acuccagccccacagccucagc

SEQ ID NO: 79 - RNA, 23 nt, Homo sapiens
aaaauggugcccuagugacuaca

SEQ ID NO: 80 - RNA, 22 nt, Homo sapiens
uauugcacuugucccggccugu

SEQ ID NO: 81 - RNA, 22 nt, Homo sapiens
accacugaccguugacuguacc

SEQ ID NO: 82 - RNA, 24 nt, Homo sapiens
gcugguuucauauggugguuuaga

SEQ ID NO: 83 - RNA, 22 nt, Homo sapiens
uuaucagaaucuccagggguac

SEQ ID NO: 84 - RNA, 23 nt, Homo sapiens
uggaagacuagugauuuuguugu

SEQ ID NO: 85 - RNA, 22 nt, Homo sapiens
uuaugguuugccugggacugag

SEQ ID NO: 86 - RNA, 22 nt, Homo sapiens
aacuggccuacaaagucccagu

SEQ ID NO: 87 - RNA, 22 nt, Homo sapiens
ggagacgcggcccuguuggagu

SEQ ID NO: 88 - RNA, 22 nt, Homo sapiens
ugggucuuugcgggcgagauga

SEQ ID NO: 89 - RNA, 22 nt, Homo sapiens
uggcucaguucagcaggaacag

SEQ ID NO: 90 - RNA, 23 nt, Homo sapiens
uaauacugccggguaaugaugga

SEQ ID NO: 91 - RNA, 23 nt, Homo sapiens
ucaagagcaauaacgaaaaaugu

SEQ ID NO: 92 - RNA, 21 nt, Homo sapiens
uugugcuugaucuaaccaugu

SEQ ID NO: 93 - RNA, 23 nt, Homo sapiens
ugguuguaguccgugcgagaaua

SEQ ID NO: 94 - RNA, 19 nt, Homo sapiens
aauggauuuuuggagcagg

SEQ ID NO: 95 - RNA, 22 nt, Homo sapiens
gauuagggugcuuagcuguuaa

SEQ ID NO: 96 - RNA, 22 nt, Homo sapiens
uaauacugccugguaaugauga

SEQ ID NO: 97 - RNA, 22 nt, Homo sapiens
uaauacugucugguaaaaccgu

SEQ ID NO: 98 - RNA, 22 nt, Homo sapiens
ucgugucuuguguugcagccgg

SEQ ID NO: 99 - RNA, 22 nt, Homo sapiens
caugccuugaguguaggaccgu

SEQ ID NO: 100 - RNA, 22 nt, Homo sapiens
uucccuuugucauccuaugccu

SEQ ID NO: 101 - RNA, 22 nt, Homo sapiens
uguaaacauccuugacuggaag

SEQ ID NO: 102 - RNA, 22 nt, Homo sapiens
aauugcacgguauccaucugua

SEQ ID NO: 103 - RNA, 23 nt, Homo sapiens
uacccuguagauccgaauuugug

SEQ ID NO: 104 - RNA, 22 nt, Homo sapiens
ccucccacacccaaggcuugca

SEQ ID NO: 105 - RNA, 22 nt, Homo sapiens
uacccauugcauaucggaguug

SEQ ID NO: 106 - RNA, 22 nt, Homo sapiens
uaacacugucugguaacgaugu

SEQ ID NO: 107 - RNA, 22 nt, Homo sapiens
aacccguagauccgaucuugug

SEQ ID NO: 108 - RNA, 22 nt, Homo sapiens
aaugcaccugggcaaggauuca

SEQ ID NO: 109 - RNA, 23 nt, Homo sapiens
cccaguguucagacuaccuguuc

SEQ ID NO: 110 - RNA, 23 nt, Homo sapiens
uaauccuugcuaccugggugaga

SEQ ID NO: 111 - RNA, 17 nt, Homo sapiens
ucucgcuggggccucca

SEQ ID NO: 112 - RNA, 22 nt, Homo sapiens
acagauucgauucuaggggaau

SEQ ID NO: 113 - RNA, 22 nt, Homo sapiens
uguaacagcaacuccaugugga

SEQ ID NO: 114 - RNA, 22 nt, Homo sapiens
caaauucguaucuaggggaaua

SEQ ID NO: 115 - RNA, 20 nt, Homo sapiens
ccccagggcgacgcggcggg

SEQ ID NO: 116 - RNA, 24 nt, Homo sapiens
aauccuuggaaccuaggugugagu

SEQ ID NO: 117 - RNA, 22 nt, Homo sapiens
uguaaacauccucgacuggaag

SEQ ID NO: 118 - RNA, 22 nt, Homo sapiens
cuuucagucggauguuuacagc

SEQ ID NO: 119 - RNA, 22 nt, Homo sapiens
acaguagucugcacauugguua

SEQ ID NO: 120 - RNA, 22 nt, Homo sapiens
uguaaacauccucgacuggaag

SEQ ID NO: 121 - RNA, 22 nt, Homo sapiens
uguaaacauccccgacuggaag

SEQ ID NO: 122 - RNA, 21 nt, Homo sapiens
augaccuaugaauugacagac

SEQ ID NO: 123 - RNA, 22 nt, Homo sapiens
aacccguagauccgaacuugug

SEQ ID NO: 124 - RNA, 22 nt, Homo sapiens
uggacggagaacugauaagggu

SEQ ID NO: 125 - RNA, 22 nt, Homo sapiens
ugaaacauacacgggaaaccuc

SEQ ID NO: 126 - RNA, 22 nt, Homo sapiens
ugauugguacgucuguggguag

SEQ ID NO: 127 - RNA, 21 nt, Homo sapiens
agggagggacgggggcugugc

SEQ ID NO: 128 - RNA, 22 nt, Homo sapiens
gugaaauguuuaggaccacuag

SEQ ID NO: 129 - RNA, 25 nt, Homo sapiens
cccccacaaccgcgcuugacuagcu

SEQ ID NO: 130 - RNA, 27 nt, Homo sapiens
auugaucaucgacacuucgaacgcaau

SEQ ID NO: 131 - RNA, 21 nt, Homo sapiens
acuggacuuggagucagaagg

SEQ ID NO: 132 - RNA, 23 nt, Homo sapiens
uacccuguagaaccgaauuugug

SEQ ID NO: 133 - RNA, 23 nt, Homo sapiens
agcugguguugugaaucaggccg

SEQ ID NO: 134 - RNA, 22 nt, Homo sapiens
ucggauccgucugagcuuggcu

SEQ ID NO: 135 - RNA, 22 nt, Homo sapiens
acuggacuuagggucagaaggc

SEQ ID NO: 136 - RNA, 21 nt, Homo sapiens
uggcagggaggcugggagggg

SEQ ID NO: 137 - RNA, 22 nt, Homo sapiens
ucccugagacccuaacuuguga

SEQ ID NO: 138 - RNA, 23 nt, Homo sapiens
cgcauccccuagggcauuggugu

SEQ ID NO: 139 - RNA, 19 nt, Homo sapiens
uggauuuuuggaucaggga

SEQ ID NO: 140 - RNA, 23 nt, Homo sapiens
uguaaacauccuacacucucagc

SEQ ID NO: 141 - RNA, 17 nt, Homo sapiens
gugggggagaggcuguc

SEQ ID NO: 142 - RNA, 22 nt, Homo sapiens
ugugcgcagggagaccucuccc

SEQ ID NO: 143 - RNA, 24 nt, Homo sapiens
uuuggcaaugguagaacucacacu

SEQ ID NO: 144 - RNA, 22 nt, Homo sapiens
acucggcguggcgucggucgug

SEQ ID NO: 145 - RNA, 22 nt, Homo sapiens
acagcaggcacagacaggcagu

SEQ ID NO: 146 - RNA, 22 nt, Homo sapiens
cugggagaaggcuguuuacucu

SEQ ID NO: 147 - RNA, 22 nt, Homo sapiens
uauggcacugguagaauucacu

SEQ ID NO: 148 - RNA, 22 nt, Homo sapiens
uagcaccauuugaaaucgguua

SEQ ID NO: 149 - RNA, 22 nt, Homo sapiens
aauccuuugucccugggugaga

SEQ ID NO: 150 - RNA, 22 nt, Homo sapiens
ucuacagugcacgugucuccag

SEQ ID NO: 151 - RNA, 20 nt, Homo sapiens
gugucugggcggacagcugc

SEQ ID NO: 152 - RNA, 22 nt, Homo sapiens
uguaaacauccuacacucagcu

SEQ ID NO: 153 - RNA, 25 nt, Homo sapiens
agggaucgcgggcggguggcggccu

SEQ ID NO: 154 - RNA, 23 nt, Homo sapiens
guccaguuuucccaggaaucccu

SEQ ID NO: 155 - RNA, 24 nt, Homo sapiens
ucccugagacccuuuaaccuguga

SEQ ID NO: 156 - RNA, 22 nt, Homo sapiens
ugaccgauuucuccugguguuc

SEQ ID NO: 157 - RNA, 23 nt, Homo sapiens
agcuacauugucugcuggguuuc

SEQ ID NO: 158 - RNA, 22 nt, Homo sapiens
aacacaccuauucaaggauuca

SEQ ID NO: 159 - RNA, 22 nt, Homo sapiens
auguagggcuaaaagccauggg

SEQ ID NO: 160 - RNA, 21 nt, Homo sapiens
aauggcgccacuaggguugug

SEQ ID NO: 161 - RNA, 22 nt, Homo sapiens
aaucguacagggucauccacuu

SEQ ID NO: 162 - RNA, 22 nt, Homo sapiens
cacccguagaaccgaccuugcg

SEQ ID NO: 163 - RNA, 22 nt, Homo sapiens
ugugacugguugaccagagggg

SEQ ID NO: 164 - RNA, 24 nt, Homo sapiens
agccugauuaaacacaugcucuga

SEQ ID NO: 165 - RNA, 23 nt, Homo sapiens
uaauccuugcuaccugggugaga

SEQ ID NO: 166 - RNA, 22 nt, Homo sapiens
uaaucucagcuggcaacuguga

(19) United States
(12) Patent Application Publication
(10) Pub. No.: US 2012/0251451 A1
(43) Pub. Date: Oct. 4, 2012

(54) Renal Cell Carcinoma Biomarkers

(76) Inventors: K.W. Michael SIU, Toronto (CA); Leroi V. Desouza, North York (CA); George M. Yousef, North York (CA); Alexander D. Romaschin, Toronto (CA)

(21) Appl. No.: 13/330,421

(22) Filed: Dec. 19, 2011

Related U.S. Application Data
(63) Continuation of application No. PCT/CA2010/000946, filed on Jun. 18, 2010.
(60) Provisional application No. 61/213,563, filed on Jun. 19, 2009.

(51) Int. Cl. G01N 21/64; A61K 49/00; C40B 40/06; C40B 30/04; G01N 21/76

(52) U.S. Cl. 424/9.1; 506/9; 435/6.11; 435/7.23; 506/16; 435/6.13

(57) ABSTRACT

The invention provides markers for renal cancer, polynucleotides encoding same, and precursors thereof. The invention also provides methods for detecting, diagnosing, screening for, monitoring, assessing, and treating renal cancers and related disease conditions in a subject. The invention further provides a method of selecting for or assessing efficacy of agents against renal cancer, and a method for assessing renal cancer cell carcinogenic potential of a compound. The invention further provides localization and imaging methods for renal cancers. Also provided are diagnostic compositions and kits for carrying out methods of the invention. In addition, the invention provides therapeutic applications for renal cancers which employ protein renal cancer markers and polynucleotides encoding same, miRNA renal cancer markers and precursors thereof, and binding agents for the markers.

### CROSS REFERENCE TO PRIOR APPLICATIONS

This application is a Continuation of PCT Application No. PCT/CA2010/000946, filed on Jun. 18, 2010, which claims priority from U.S. Provisional application No. 61/213,563, filed Jun. 19, 2009. The disclosures of the aforementioned related applications are incorporated herein by reference in their entirety.

### FIELD OF THE INVENTION

The invention relates to renal cancer markers, methods for assessing the status of renal cell tissue, and methods for the detection, diagnosis, prediction, and therapy of renal cancer. One aspect of the invention relates to biomarkers of renal cell carcinoma and methods for detection, diagnosis, prediction, and therapy for renal cell carcinoma and related conditions.

### BACKGROUND OF THE INVENTION

Kidney cancer is a common urologic malignancy that accounts for about 3% of adult malignancies (1a) and causes about 90,000 deaths worldwide annually. Renal cell carcinoma (RCC) is the most common neoplasm in the adult kidney, representing 3% of all adult malignancies (2). The incidence of RCC has steadily increased over the past 20 years. Histopathologically, about 80% of RCCs are clear-cell RCCs, 15% are papillary, and the remaining 5% represent other types. Early diagnosis of kidney-localized RCC is associated with a fairly favorable prognosis: a five-year survival rate of about 89%. Unfortunately, patients often present with few signs, symptoms, or laboratory abnormalities, and are frequently (−30%) diagnosed at the metastatic stage, when the five-year survival rate is significantly lowered (9% five-year survival rate) (3). The clinical diagnosis of RCC is often confirmed by imaging studies, including X-ray and computed tomography. The possible existence of non-neoplastic mass lesions can be a serious challenge to the diagnosis of RCC, and there are currently no biomarkers available for the diagnosis of RCC. Apart from surgery, few treatment options are available because RCC is both chemotherapy- and radiotherapy-resistant.

Heretofore, biomarkers for early detection and follow-up of the disease have not been available, accounting for late diagnosis and subsequent poor prognosis. A clearer understanding of the pathogenesis of RCC is required for developing new target therapies and biomarkers that predict treatment efficacy.

Molecular expression studies of cancer provide opportunities to identify new cancer biomarkers and improve cancer detection and disease management (4). In addition to early detection, biomarkers have potential uses in prognosis and monitoring (e.g., monitoring of disease-state progress and recurrence after treatment), and as predictive markers for optimal treatment modality. Ascertaining optimal treatment options is important, as it may help patients to avoid unnecessary treatments that may be ineffective or pose serious side effects (5).

Molecular-expression studies, especially those involving quantitative, differential-expression analyses, are useful in providing insight into the changes in functional pathways leading to cancer, thereby elucidating the mechanisms of cancer development at the molecular level (6). These studies can provide the basis for identifying new molecular drug targets for therapy.

Proteomics play a significant role in the study of cancer because proteins contribute heavily to malignant phenotypes. Mass-spectrometry-based protein expression examinations have been successfully used to identify and evaluate new biomarkers for cancer, including prostate (7), endometrial (8-10), breast (11), pancreatic (12), and head-and-neck (13, 14) cancers, among many others (4). Proteomics techniques, combined with mass spectrometry (MS), offer great promise for unveiling the complex molecular events of tumorigenesis and identifying cancer biomarkers. In particular, differential tagging with isotopic reagents—e.g., isotope-coded affinity tags (ICAT) (1) or the more recent variation that uses isobaric tagging reagents, iTRAQ (Applied Biosystems, Foster City, Calif.)—followed by multidimensional liquid chromatography (LC) and tandem mass spectrometry (MS/MS) analysis can provide a powerful methodology in the search for biomarkers for various diseases.

A class of small, non-coding RNAs, called microRNAs (miRNAs), has also been identified as key regulators in many biological processes including cellular development, differentiation, apoptosis and proliferation. miRNAs regulate gene expression at the post-transcriptional level by binding, through partial sequence homology, to the 3′ untranslated region (3′ UTR) of mammalian target mRNAs, and causing translational inhibition and/or mRNA degradation (2a). Mature miRNAs are 18-25 nucleotides long, and are the result of sequential processing of primary transcripts (pri-miRNAs) mediated by a complex protein system that includes two RNase III enzymes, Drosha and Dicer, members of the Argonaute family, and Pol II-dependent transcription (3a). The pre-miRNA precursor is cleaved by cytoplasmic RNase III endonuclease Dicer into a 22-nucleotide mature double-stranded miRNA. The strand which serves as mature miRNA is incorporated into the RNA-induced silencing complex (RISC) and drives the selection of target mRNAs containing antisense sequences, thereby controlling target gene expression (4a).

It has been shown that miRNAs are aberrantly expressed or mutated in cancers, suggesting that they may play a role as a novel class of oncogenes or tumor suppressor genes (5a,6a). miRNAs have exhibited differential expression in a variety of cancers, including prostate, lung, breast, colon, and other malignancies (7a). Recent evidence has shown diverse clinical applications of miRNAs in respect of the diagnosis, prognosis, and prediction of cancers (8a).

### SUMMARY OF THE INVENTION

As described in greater detail herein, the inventors have identified markers associated with renal cell cancer, including renal cell carcinoma. The inventors used multidimensional liquid chromatography-mass spectrometry (LC-MS/MS) for the analysis of biological samples labelled with isobaric mass tags (iTRAQ) to identify proteins that are differentially expressed in renal cell carcinoma (RCC) in relation to adjacent normal counterparts obtained from nephrectomy specimens (control) for cancer biomarker discovery. Two kidney cancer samples were compared against a non-cancerous diseased kidney and normal kidney by online and offline separation. Nine hundred and thirty seven (937) proteins were identified in RCCs, including structural proteins, signalling components, enzymes, receptors, transcription factors and chaperones. Using cutoff values of 1.5 fold for overexpression, and 0.67 fold for underexpression, the inventors were able to identify 168 underexpressed proteins, and 156 proteins that were overexpressed in RCC compared to their normal tissues. These cutoff values were used by the inventors for selecting proteins for further statistical analyses in previous studies (Ralhan et al. 2008) and were found to perform satisfactorily.

Accordingly, in one aspect, the present invention provides a method for detecting one or more renal cancer markers in a subject by: (a) obtaining a sample from a subject; (b) detecting in the sample an amount of (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof; and (c) comparing the detected amount with an amount detected for a standard.

In another aspect, the present invention provides a method for diagnosing renal cancer in a subject, by comparing: (a) levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, detected in a sample from the subject; and (b) normal levels of expression of the corresponding renal cancer marker or polynucleotide in a control sample, wherein a significant difference in levels of the renal cancer marker, relative to the corresponding normal levels, is indicative of the renal cancer in the subject.

In certain embodiments of the present invention, the renal cancer marker is a protein. In other embodiments of the present invention, the renal cancer marker is miRNA.

In yet another aspect, the present invention provides a method for diagnosing renal cancer in a subject, by: (a) contacting a biological sample obtained from the subject with at least one binding agent that specifically binds to (i) at least one of the renal cancer markers listed in Table 8, or a part thereof, (ii) at least one polynucleotide encoding at least one of the renal cancer markers listed in Table 8, or a part thereof, or (iii) at least one of the renal cancer markers listed in Table 12 or a precursor thereof, or a part thereof; and (b) detecting in the sample amounts of the renal cancer marker or polynucleotide or part thereof that binds to the at least one binding agent, relative to a predetermined standard or cut-off value, and thereby determining the presence or absence of the renal disease in the subject. In one embodiment, the binding agent is an antibody.

In another aspect, the present invention provides a method of screening a subject for renal cancer by: (a) obtaining a biological sample from a subject; (b) detecting in the sample an amount of (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof; and (c) comparing the detected amount of the renal cancer marker or polynucleotide with a predetermined standard, wherein detection of a level of the renal cancer marker or polynucleotide different from that of the standard is indicative of renal cancer.

In embodiments of the invention, the level of the renal cancer marker or polynucleotide is significantly higher than the standard and is indicative of renal cancer. In certain other embodiments, the level of the renal cancer marker or polynucleotide is significantly lower than the standard and is indicative of renal cancer. In further embodiments, the sample is obtained from a tissue, extract, cell culture, cell lysate, lavage fluid, or physiological fluid of the subject. In still other embodiments, the sample is obtained from renal tumor tissue.

In a further aspect, the present invention provides a method for determining the presence or absence of a renal cancer marker associated with a renal disease in a subject, by: (a) detecting: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in a sample from the subject; and (b) relating the detected amount of the marker or polynucleotide to the presence of the renal disease. In one embodiment, the polynucleotide is mRNA. In another embodiment, the polynucleotide, Table 12 marker, or precursor thereof is detected by: (a) contacting the sample with at least one oligonucleotide that hybridizes to the polynucleotide, Table 12 marker, or precursor thereof; and (b) detecting in the sample levels of at least one nucleic acid that hybridizes to the polynucleotide, Table 12 marker, or precursor thereof, relative to a predetermined standard or cut-off value, and thereby determining the presence or absence of the renal disease in the subject.

In one embodiment, the polynucleotide, Table 12 marker, or precursor thereof is detected with an amplification reaction. In other embodiments, the amplification reaction includes a polymerase chain reaction employing oligonucleotide primers that hybridize to the polynucleotide, Table 12 marker, or precursor thereof or to a complement thereof. Additionally, in certain embodiments, the polynucleotide, Table 12 marker, or precursor thereof is detected with at least one oligonucleotide probe that hybridizes to the polynucleotide, Table 12 marker, or precursor thereof or to complement thereof.

In a further embodiment, the polynucleotide, Table 12 marker, or precursor thereof is detected by: (a) isolating RNA from the sample; (b) combining the RNA with at least one reagent, to convert the RNA to cDNA; (c) treating the cDNA with at least one amplification reaction reagent and at least one primer that hybridizes to the cDNA, to produce at least one amplification product; (d) analyzing the at least one amplification product to detect an amount of RNA encoding the at least one renal cancer marker; and (e) comparing the amount of RNA to an amount detected against a panel of expected values for normal tissue derived using similar primers.

In another aspect, the present invention provides a method for diagnosing and monitoring renal cancer in a subject by: (a) isolating at least one nucleic acid in a sample from the subject; and (b) detecting (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, wherein the presence of higher or lower levels of the marker or polynucleotide in the sample, compared to a standard or control, is indicative of the disease or prognosis.

In yet another aspect, the present invention provides a method for monitoring the progression of renal cancer in a subject, by: (a) detecting in a sample from the subject, at a first time point, (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof; (b) repeating step (a) at a subsequent point in time; and (c) comparing levels detected in steps (a) and (b), and thereby monitoring the progression of renal cancer.

In a further aspect, the present invention provides a method for determining in a subject whether renal cancer has metastasized or is likely to metastasize in the future by comparing: (a) levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in a sample from a subject; and (b) normal levels or non-metastatic levels of the renal cancer marker or polynucleotide in a control sample, wherein a significant difference between the levels in the subject sample and the normal levels or non-metastatic levels is indicative that the renal cancer has metastasized.

In yet another aspect, the present invention provides a method for assessing the aggressiveness or indolence of renal cancer, by comparing: (a) levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in a sample from a subject; and (b) normal levels of the renal cancer marker or polynucleotide in a control sample, wherein a significant difference between the levels in the subject sample and normal levels is indicative that the cancer is aggressive or indolent.

In still another aspect, the present invention provides a diagnostic composition including an agent that: binds to at least one of the renal cancer markers listed in Table 8, hybridizes to at least one polynucleotide encoding at least one of the renal cancer markers listed in Table 8, or (iii) binds to at least one of the renal cancer markers listed in Table 12 or a precursor thereof.

In a further aspect, the present invention provides a method for assessing the potential efficacy of a test agent for inhibiting renal cancer in a subject, by comparing: (a) levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in a first sample obtained from the subject and exposed to the test agent; and (b) levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in a second sample obtained from the subject, wherein the second sample has not been exposed to the test agent, wherein a significant difference in the levels of the renal cancer marker or polynucleotide in the first sample, relative to the second sample, is an indication that the test agent inhibits renal cancer in the subject.

In yet another aspect, the present invention provides a method for assessing the efficacy of a therapy for inhibiting renal cancer in a subject, by comparing: (a) levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in a first sample obtained from the subject; and (b) levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in a second sample obtained from the subject following therapy, wherein a significant difference in the levels of expression of the renal cancer marker or polynucleotide in the second sample, relative to the first sample, is an indication that the therapy inhibits renal cancer in the subject.

In one aspect, the present invention provides a method for selecting an agent for inhibiting renal cancer in a subject, by: (a) obtaining a sample including precancer or cancer cells from the subject; (b) separately exposing aliquots of the sample to a plurality of test agents; (c) comparing levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in the aliquots; and (d) selecting one of the test agents which alters the levels of the renal cancer marker or polynucleotide in the aliquot containing that test agent, relative to other test agents.

In another aspect, the present invention provides a method for inhibiting renal cancer in a subject, by: (a) obtaining a sample including precancer or cancer cells from the subject; (b) separately maintaining aliquots of the sample in the presence of a plurality of test agents; (c) comparing levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in the aliquots; and (d) administering to the subject at least one of the test agents which alters the levels of the renal cancer marker or polynucleotide in the aliquot containing that test agent, relative to other test agents.

In yet another aspect, the present invention provides a method for assessing the renal cancer cell carcinogenic potential of a test compound, by: (a) maintaining separate aliquots of renal cancer cells in the presence and absence of the test compound; and (b) comparing levels of: (i) at least one of the renal cancer markers listed in Table 8, (ii) at least one polynucleotide encoding at least one of the renal cancer markers listed in Table 8, or (iii) at least one of the renal cancer markers listed in Table 12 or a precursor thereof, in the aliquots, wherein a significant difference in levels of the renal cancer marker or polynucleotide in the aliquot maintained in the presence of the test compound, relative to levels in the aliquot maintained in the absence of the test compound, is indicative that the test compound possesses renal cancer cell carcinogenic potential.

In a further aspect, the present invention provides an in vivo method for imaging a renal disease, by: (a) injecting a subject with one or more agent that binds to a renal cancer marker listed in Table 8, the agent carrying a label for imaging the renal cancer marker; (b) allowing the agent to incubate in vivo and bind to a renal cancer marker; and (c) detecting the presence of the label localized to diseased kidney tissue. In one embodiment, the agent is an antibody that specifically reacts with a renal cancer marker.

In certain embodiments of the invention, the at least one renal cancer marker is listed in Table 8. In other embodiments, the at least one renal cancer marker is listed in Table 12 or a precursor thereof.

In one aspect, the present invention provides a set of renal cancer markers, including at least 2 of the markers listed in Table 8. In one embodiment, the at least 2 markers include YWHAH, CAPNS1, KNG1, and/or SERPING1.

In another aspect, the present invention provides a set of renal cancer markers, including at least 2 of the markers listed in Table 12 or a precursor thereof. In certain embodiments, the at least 2 markers include mir-34a, miR-155, miR-200c, miR-210, and/or miR-1974.

In a further aspect, the present invention provides kits comprising the renal cancer markers, polynucleotides, and precursors of the invention. By way of example, the present invention provides a kit for determining the presence of renal cancer in a subject, including a known amount of at least one binding agent that binds to (i) at least one of the renal cancer markers listed in Table 8, or a part thereof, (ii) at least one polynucleotide encoding at least one of the renal cancer markers listed in Table 8, or a part thereof, or (iii) at least one of the renal cancer markers listed in Table 12 or a precursor thereof, or a part thereof, wherein the binding agent includes a detectable substance or binds directly or indirectly to a detectable substance.

In yet another aspect, the present invention provides a kit for determining the presence of renal cancer in a subject, including a known amount of an oligonucleotide that hybridizes to (a) a polynucleotide encoding a renal cancer marker listed in Table 8, or (b) a miRNA renal cancer marker listed in Table 12 or a precursor thereof, wherein the oligonucleotide is directly or indirectly labelled with a detectable substance.

In other embodiments of the present invention, the renal cancer marker is a renal cell carcinoma (RCC) marker. In further embodiments of the present invention, the renal cancer markers is a clear-cell renal cell carcinoma (ccRCC) marker.

Other objects, features, and advantages of the present invention will become apparent from the following detailed description. It should be understood, however, that the detailed description, and the specific examples which indicate preferred embodiments of the invention, are given by way of illustration only, since various changes and modifications within the spirit and scope of the invention will become apparent to those skilled in the art from the detailed description which follows.

### BRIEF DESCRIPTION OF THE TABLES AND DRAWINGS

### Tables

Table 1 is a distribution of proteins and their expression levels in RCC cells compared to their normal counterparts.

Table 2 is a partial list of differentially expressed proteins in kidney cancer tissue compared to normal counterparts from the same patient.

Table 3 shows the protein expression in RCC sample compared to non-cancerous renal failure and transitional cell carcinoma (TCC).

Table 4 shows dysregulated proteins in the inventors' analysis that were found to be differentially expressed in kidney cancer by other independent reports.

Table 5 is a partial list of proteins from the inventors' analysis with documented kidney expression in the SwissProt database.

Table 6 is a comparison between protein and SAGE mRNA expression data for upregulated proteins that were detected in both runs.

Table 7 is a partial list of consistently overexpressed proteins (in both runs) in RCC compared to two different in silico EST databases.

Table 8 is a list of novel differentially expressed proteins in RCC tissue compared to normal counterparts from the same patient.

Table 9 is a list of the top 35 most significantly dysregulated miRNAs in clear-cell RCC compared to normal counterparts.

Table 10 is a partial list of the differentially expressed miRNAs in clear-cell RCC, their association with other types of cancer, and their target proteins.

Table 11 shows hypoxia-inducible miRNAs that are dysregulated in clear-cell RCC.

Table 12 is a complete list of 166 miRNAs differentially expressed in clear-cell RCC compared to normal counterparts.

### FIGURES

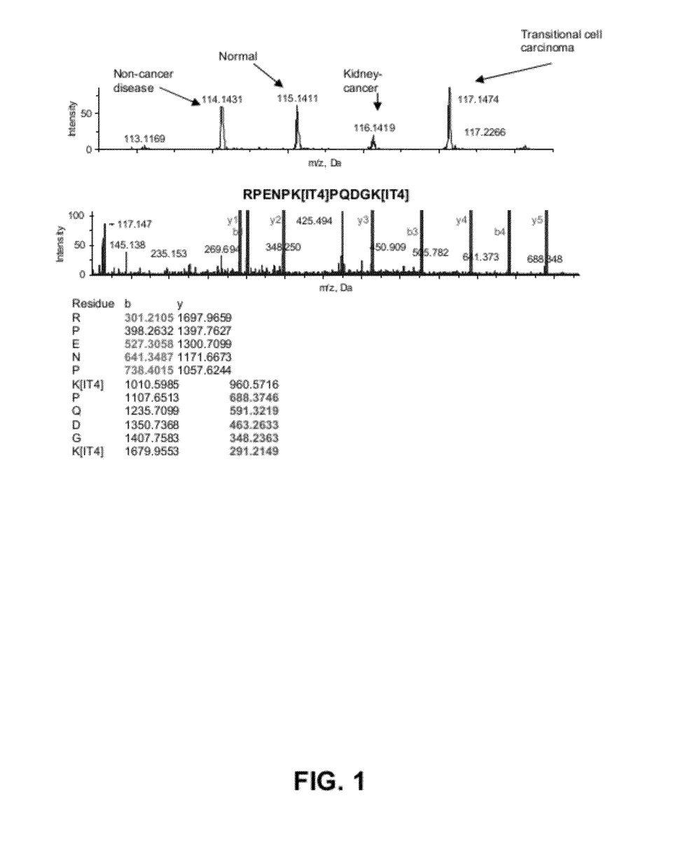

FIG. 1 is a representative spectra showing the reporter ion region (top panel) and the full MS/MS spectrum (bottom panel) for a tryptic peptide of a protein that is differentially expressed in only the kidney cancer sample versus the normal control (Protein #212: Signature ions for DNA binding protein B). The highlighted m/z values show b and y product ions that have been matched to the identified peptide sequence.

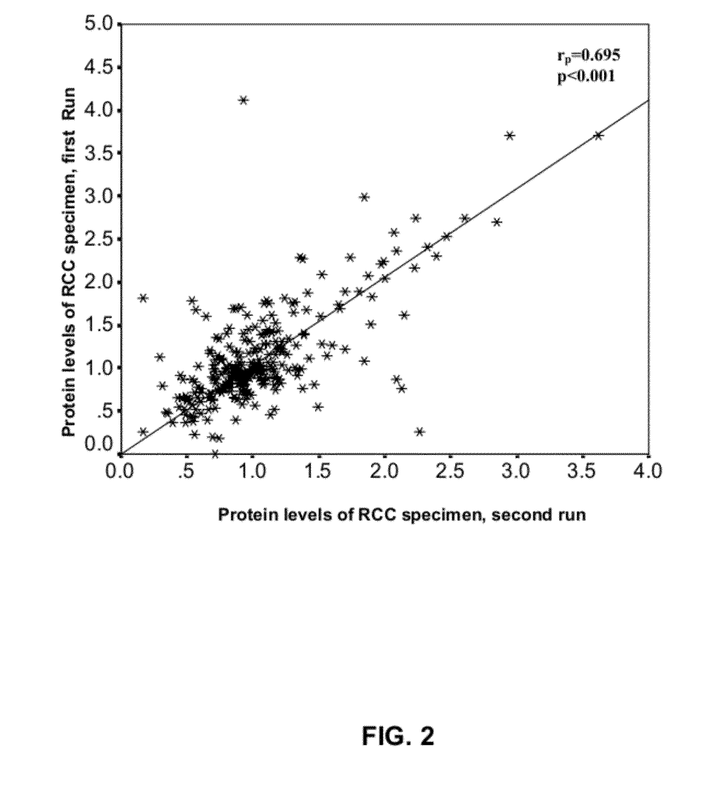

FIG. 2 shows the correlation between normalized protein expression levels in the RCC specimen between the two runs. Protein expression levels were normalized by the expression levels in normal kidney tissue. A statistically significant positive correlation was observed. Values were compared using the Pearson correlation coefficient.

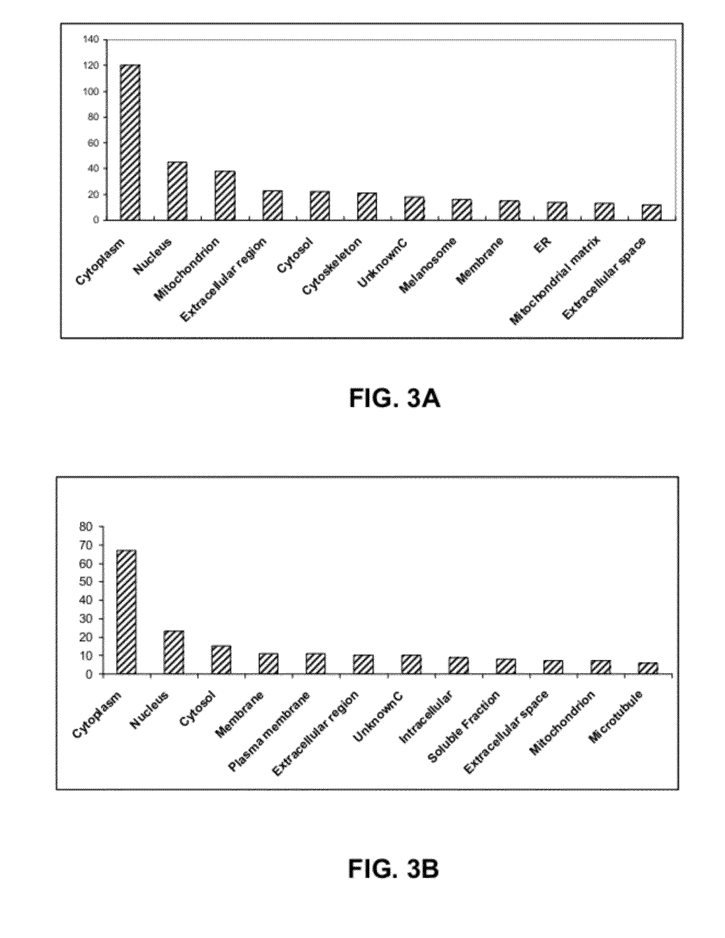

FIG. 3 shows the subcellular localization of proteins identified in RCC, as analyzed through the Gene Ontology consortium databases. This included analysis of (A) the total numbers of proteins extracted, and (B) upregulated proteins in RCC compared to their normal counterparts.

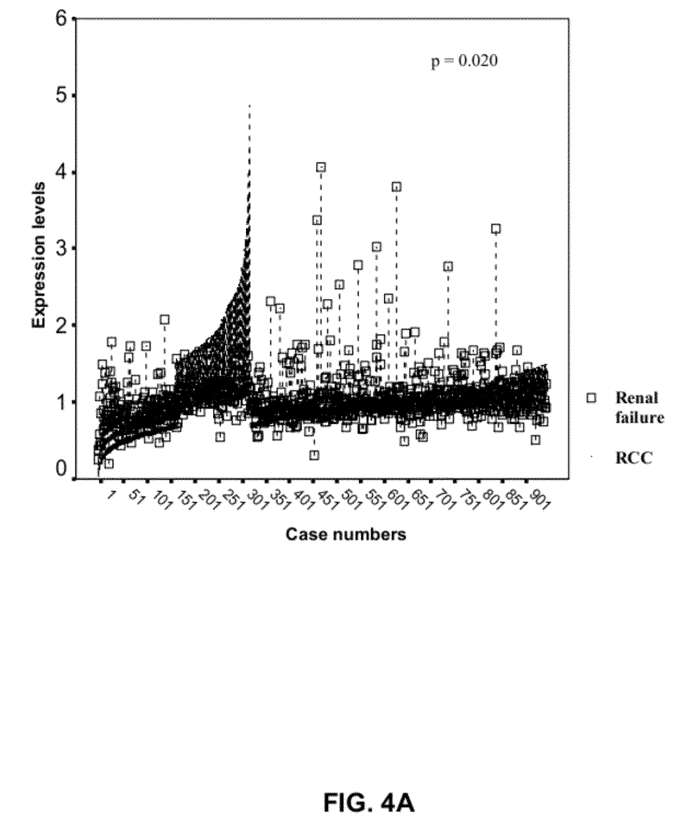

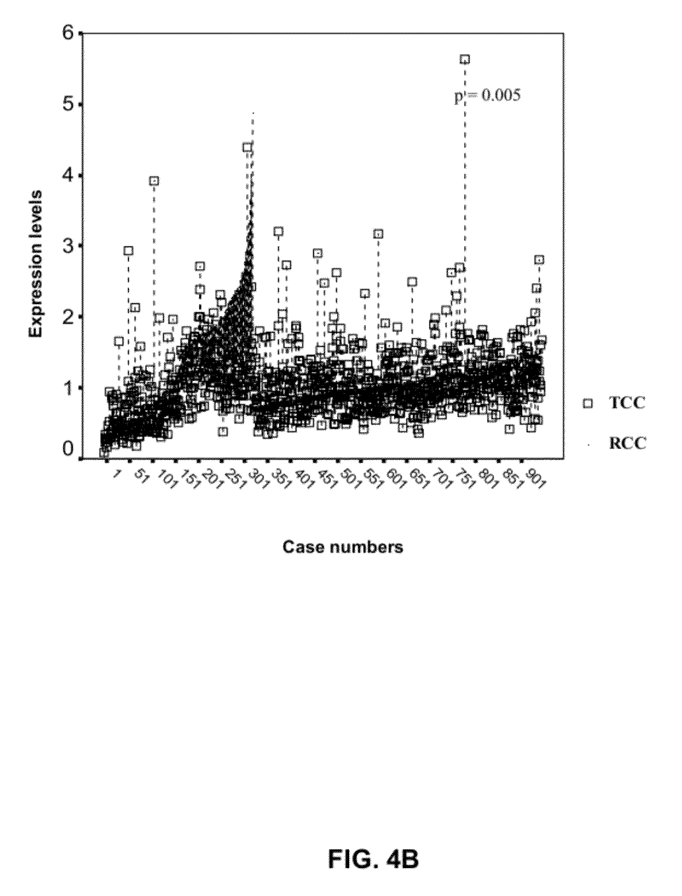

FIG. 4 shows a comparison between normalized (expressed as fold changes of normal kidney tissue expression levels) protein average expression levels in RCC to non-cancerous renal failure (FIG. 4A) and transitional cell carcinoma (TCC) (FIG. 4B) differences were statistically significant in both cases. Analysis was performed by the Wilcoxon Signed Ranks Test.

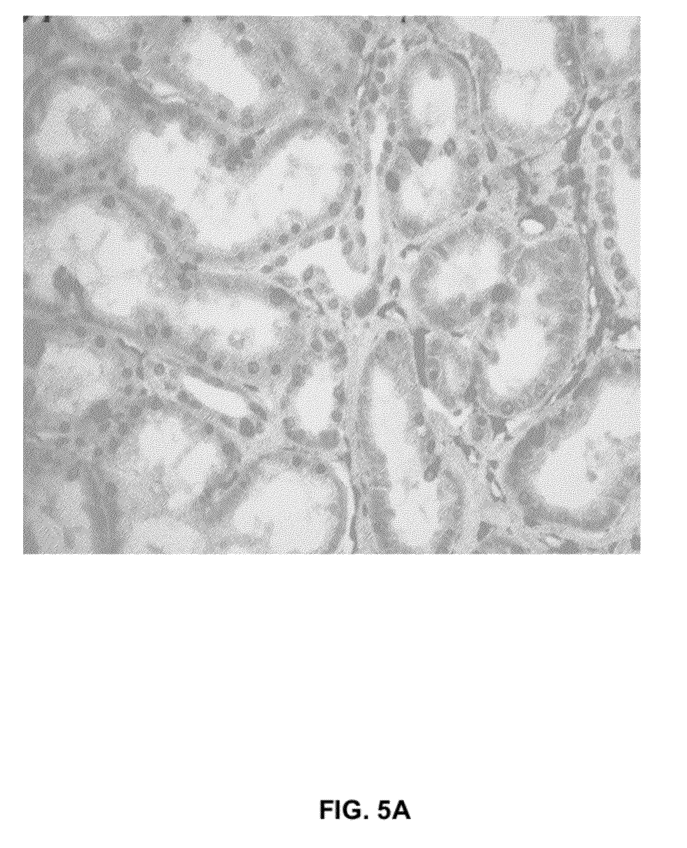

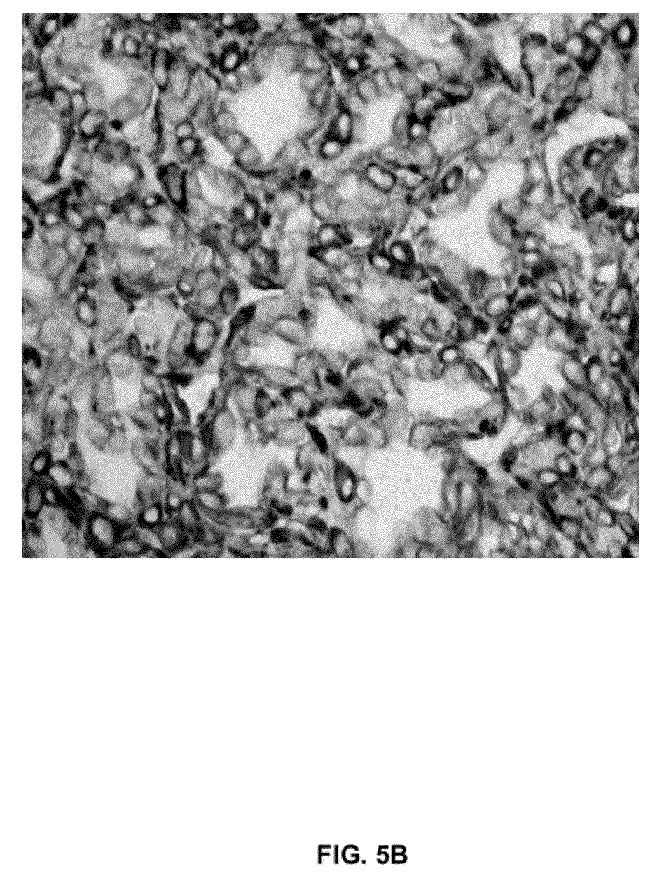

FIG. 5 shows immunohistochemistry validation of protein expression using antibodies to Vimentin in normal kidney tissue (FIG. 5A) and clear-cell RCC (FIG. 5B). Staining was minimally presence in the normal tubules whereas significant staining was observed in the cancerous tissue from the same patient confirming protein overexpression in RCC.

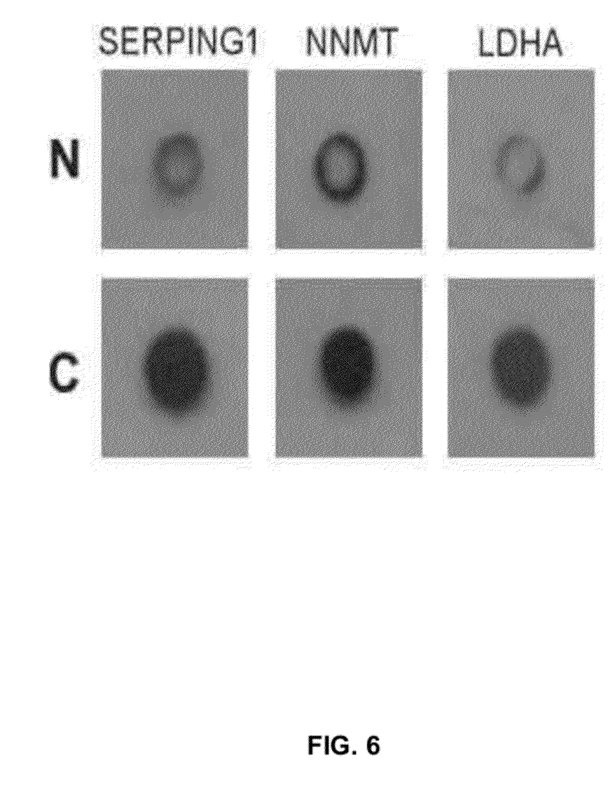

FIG. 6 is a representative dot-blot validation of protein expressions of three highly overexpressed proteins in normal tissue (N) versus clear-cell RCC tissue (C). Luminescence signals were significantly increased in the cancer sample.

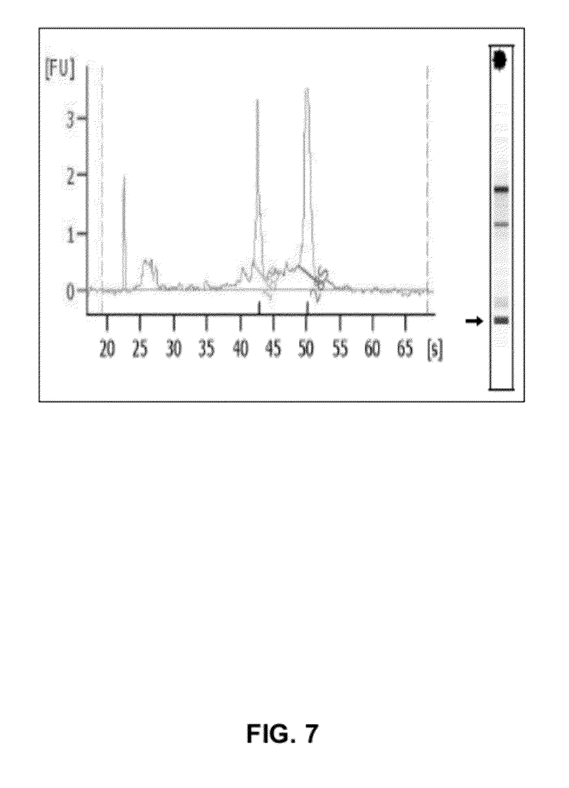

FIG. 7 is a gel analysis of total RNA extraction from kidney tissue. The arrow highlights the band on the gel (right) that represents miRNA. The graph to the left of the gel is a bio-analysis graph of the RNA extraction sample, where 18S and 28S are used as the inventors' quality standard.

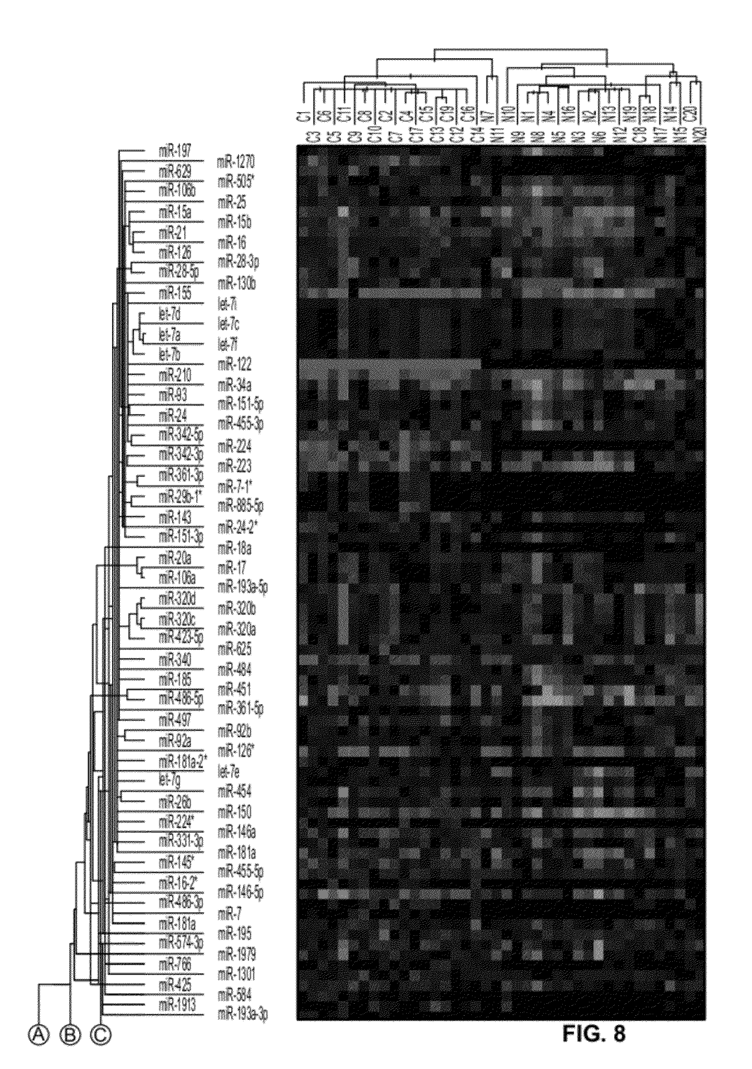

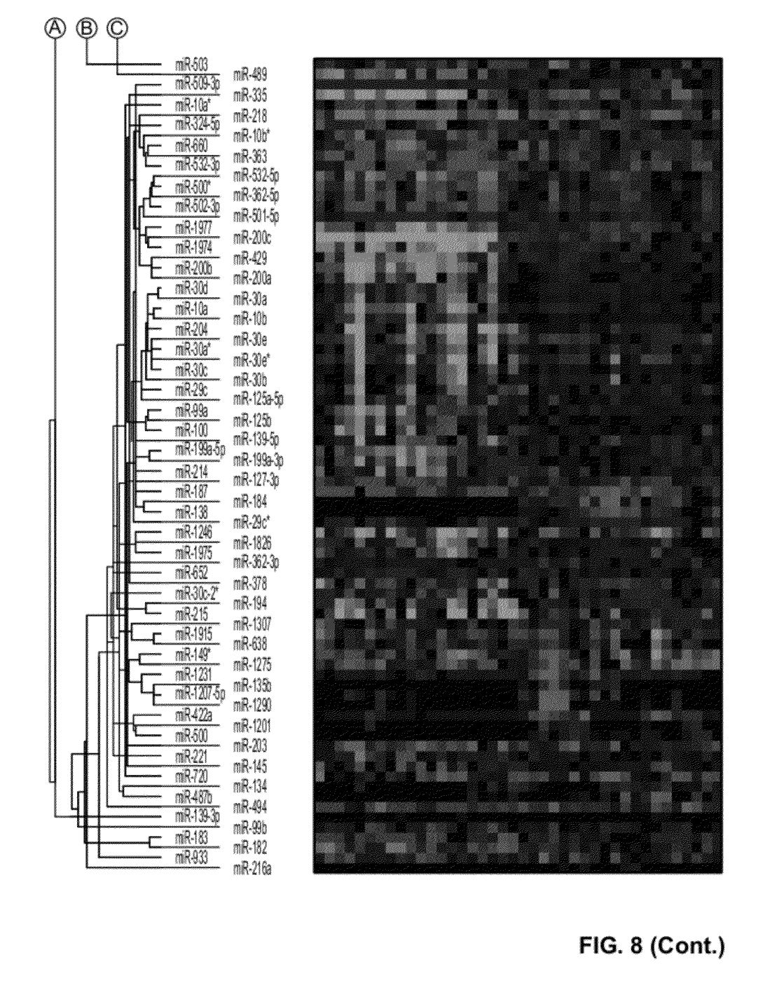

FIG. 8 is a microarray heat map showing the 166 statistically significant (p<0.05) dysregulated miRNAs in RCC.

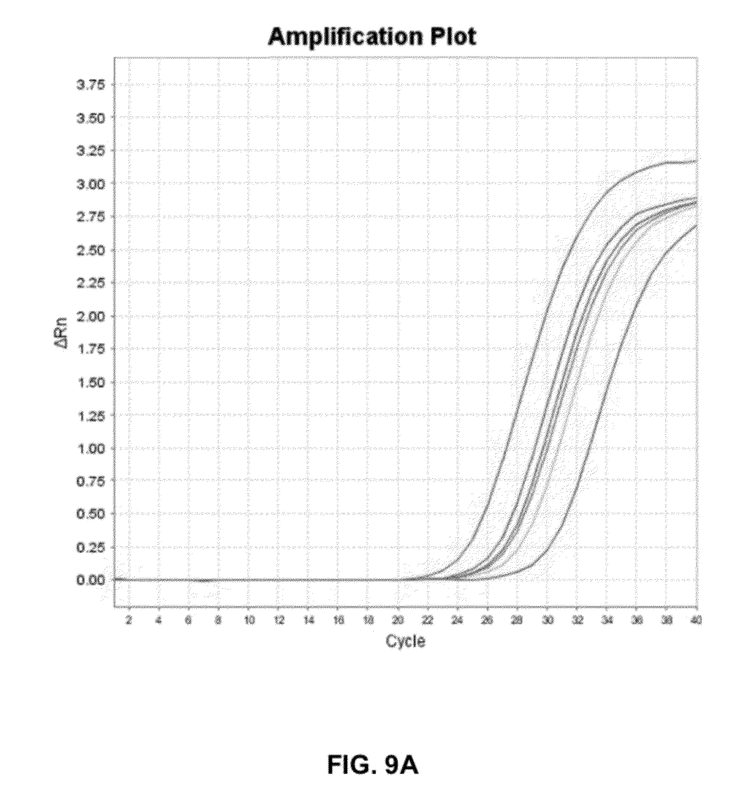

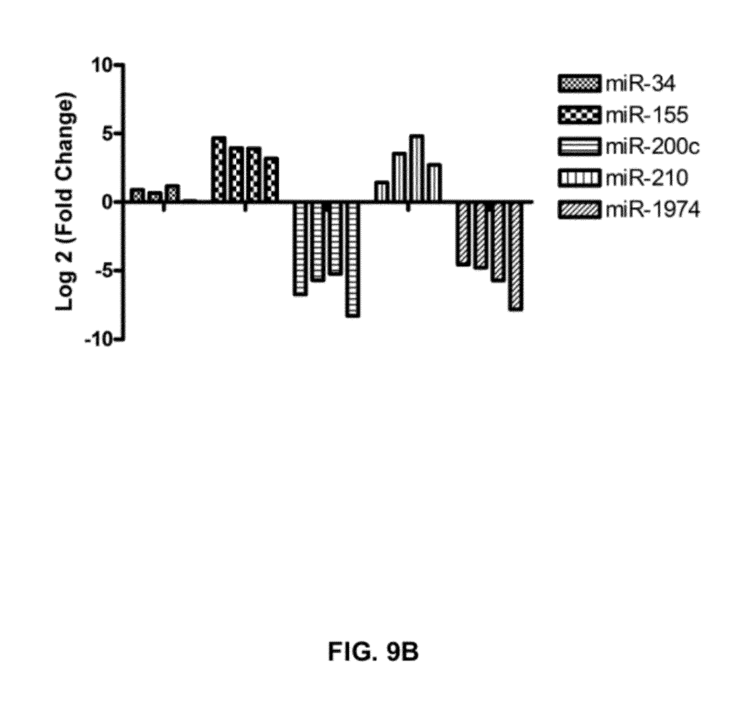

FIG. 9 depicts the quantitative RT-PCR validation of the expression of miR-34a, miR-155, miR-200c, miR-210, and miR-1974 kidney cancer when compared to normal kidney tissue in a representative sample of five patients. A. Graphic representation of the RT-PCR, in which each line denotes the miRNA and type of tissue (cancer or normal). B. Bar graph of the log 2 value of the fold change in cancer versus normal (normalized with the control, RNU44) of the five miRNAs in five patients. A positive value denotes an increase in cancer, while a negative value denotes a decrease.

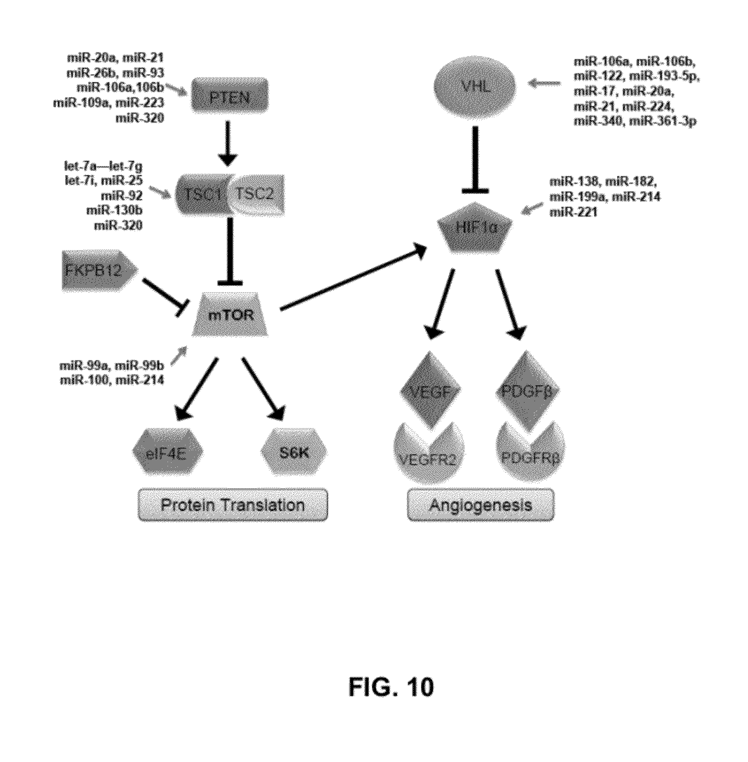

FIG. 10 shows the potential involvement of dysregulated miRNAs in renal cell cancer pathogenesis. Target prediction analysis by multiple algorithms identified, as potential targets of miRNAs dysregulated in ccRCC, certain proteins that are documented to participate in the pathogenesis of RCC.

### DETAILED DESCRIPTION OF THE INVENTION

The inventors used iTRAQ labeling in combination with multidimensional LC-MS/MS analysis of renal cancer for comparison of protein profiles of RCC and non-cancerous renal tissues in an attempt to identify potential biomarkers. The iTRAQ experiments were performed on resected RCC and non-cancerous tissue homogenates. The rationale for using whole tissue homogenates, rather than laser capture microdissection (LCM)-procured tumor cells, has been previously discussed (9, 14, 18). There are major advantages to the use of tissue homogenates. For example, the relevant proteins are much more abundant in the tissues of interest than in bodily fluids, and one can easily link the protein that is differentially expressed and the tumor itself. This type of link would need to be demonstrated if the differentially expressed proteins were to be discovered in a bodily fluid, such as blood, as many tissues or organ can potentially discharge into blood. Furthermore, the tumor microenvironment plays an important role in cancer progression (19), and the examination of protein expression in tissues from a homogenate of different cell types can take into account the contributions of the tumor microenvironment.

As described in greater detail herein, the protein expression profiles of RCCs were compared with non-cancerous renal tissues (controls) using the iTRAQ-labelling technique in combination with multidimensional LC-MS/MS analysis. iTRAQ technology places tags on primary amines, and thus potentially allowing the tagging of most tryptic peptides. The multiplexing ability afforded by the iTRAQ reagents, which are available in four different tags, is ideally suited for the comparison of cancerous and non-cancerous cells and tissues, as it provides a means to perform a proteomic analysis of both paired and non-paired non-cancerous renal tissues, while simultaneously comparing them against the cancer samples. Some of the overexpressed proteins that the Applicants identified in the tissues by the iTRAQ technology and LC-MS/MS analysis were confirmed by immunohistochemistry and dot blot analysis. These approaches ensure that the proteins selected demonstrate a consistent pattern of overexpression in RCCs and greatly increase the confidence of the observations stemming from iTRAQ analysis. Besides their potential utility as biomarkers for RCC, these proteins also provide valuable insight into the previously unknown molecular networks and mechanisms that govern the normal-to-malignant conversion of the renal cells.

In addition to proteomics studies, increased understanding of gene expression and translation regulation has permitted the inventors to determine cancer biomarkers that are nucleic acids, rather than proteineaous molecules. miRNA microarray studies allow the inventors to analyze miRNA expression profile in the clear cell subtype of RCC compared to normal counterparts. Microarray results are validated by performance of quantitative RT-PCR for the top dysregulated miRNAs. This was then followed by a bioinformatics analysis to examine their potential contribution to the pathogenesis of RCC, future clinical significance, and their correlation with known chromosomal aberration sites in RCC.

In an aspect, the invention provides marker sets that distinguish the renal diseases and uses therefor. A marker set may include a plurality of polypeptides, miRNA, polynucleotides encoding such polypeptides including or consisting of at least one marker listed in Table 8 or Table 12, as applicable, and optionally 2, 4, 5, 6, and 7 or up to all of the markers listed therein. In specific aspects, the markers consist of at least 2, 3, 4, or 5 polypeptides listed in Table 8. In another aspect, the markers consist of at least 2, 3, 4, or 5 miRNA listed in Table 12 or a precursor thereof. In a further aspect of the invention, the protein marker sets include or consist of protein clusters, or proteins in pathways including markers listed in Table 8. In another aspect of the invention, the nucleic acid markers sets include or consist of miRNA clusters, or miRNA known to involved in pathways including markers listed in FIG. 10. In one aspect the invention provides markers in Table 8 or Table 12 or Table 10 that are up-regulated or downregulated or expressed in cancer samples as compared to the non-cancer samples.

The up-regulated and down-regulated protein markers identified in Table 8 (up-regulated or down-regulated in cancer samples versus non-cancer samples), including but not limited to native-sequence polypeptides, isoforms, chimeric polypeptides, all homologs, fragments, and precursors of the protein markers, including modified forms of the polypeptides and derivatives are referred to and defined herein as “protein renal cancer marker(s)”. Polynucleotides encoding protein renal cancer markers are referred to and defined herein as “protein renal cancer polynucleotide marker(s)”, “polynucleotides encoding protein renal cancer markers”, or “polynucleotides encoding the protein cancer marker(s)”. The dysregulated miRNA markers identified in Table 12 and more specifically in Table 9 (up-regulated or down-regulated in cancer samples versus non-cancer samples), including without limitation native-sequence miRNA, isoforms, all homologs, fragments, and precursor, including modified forms of the miRNA and derivatives are referred to and defined as “miRNA renal cancer markers”. “Polynucleotide encoding the markers” include “protein renal cancer polynucleotide marker(s)”, “polynucleotides encoding the protein marker(s)”, “polynucleotides encoding a protein renal cancer marker”. The protein renal cancer markers, the polynucleotides encoding for the renal cancer markers, miRNA renal cancer markers, and are sometimes collectively referred to herein as “marker(s)” or “renal cancer marker(s)”.

Protein renal cancer markers listed in Table 8 (in cancer sample versus non-cancer sample), polynucleotide encoding for the renal cancer markers, and miRNA renal cancer markers or precursors thereof (listed in Table 12) may be used to determine the status of the renal cell or tissue and in the detection of a renal disease such as renal cancer. Thus, the markers can be used for diagnosis, monitoring, including without limitation monitoring progression of disease state or success of therapeutic treatment, prognosis, treatment, or classification of a renal disease, including without limitation cancer, RCC or related conditions, or as markers to evaluate disease state before surgery or after relapse. The invention also contemplates methods for assessing the status of a renal tissue, and methods for the diagnosis and therapy of a renal disease.

In accordance with another aspect of the invention, renal cancer can be assessed or characterized, for example, by detecting the presence in the sample of: (a) a renal cancer marker or fragment thereof; (b) a metabolite which is produced directly or indirectly by a renal cancer marker; (c) a transcribed nucleic acid or fragment thereof having at least a portion with which a protein renal cancer polynucleotide marker is substantially identical; (d) a transcribed nucleic acid or fragment thereof having at least a portion with which a miRNA renal cancer marker is substantially identical; and/or (e) a transcribed nucleic acid or fragment thereof, wherein the nucleic acid hybridizes with polynucleotide marker encoding for a protein renal cancer marker or a miRNA renal cancer marker.

The levels of renal cancer markers may be determined by methods as described herein and generally known in the art. The expression levels of protein renal cancer markers may be determined by isolating and determining the level of nucleic acid transcribed from each protein renal cancer polynucleotide marker. Alternatively or additionally, the levels of protein renal cancer markers translated from mRNA transcribed from a renal cancer polynucleotide marker may be determined. The levels of miRNA cancer markers may be determined by isolating and determining the level of each miRNA cancer marker. In an aspect, the invention provides a method for characterizing or classifying a renal sample by detecting a difference in the expression of a first plurality of renal cancer markers relative to a control, the first plurality of such markers including at least 2, 3, 4, or 5 of the renal cancer markers corresponding to the markers listed in Table 8 or Table 12, and optionally 2, 4, 5, 6, and 7, or up to all of the markers listed therein or those listed in Table 8 or Table 12 or those that are up-regulated in cancer versus control tissue as indicated in Table 8 or Table 12.

In an aspect of the invention, a method is provided for characterizing a renal tissue by detecting renal cancer markers associated with a renal tissue stage or phase, or renal disease in a subject by: (a) obtaining a sample from a subject; (b) detecting or identifying in the sample an amount of renal cancer markers; and (c) comparing the detected amount with an amount detected for a standard.

In an embodiment of the invention, a method is provided for detecting protein renal cancer markers or protein renal cancer polynucleotide markers associated with renal cancer in a patient by: (a) obtaining a sample from a patient; (b) detecting in the sample amount of protein renal cancer markers or protein renal cancer polynucleotide markers; and (c) comparing the detected amount with an amount detected for a standard.

In an embodiment of the invention, a method is provided for detecting miRNA renal cancer markers associated with renal cancer in a patient by: (a) obtaining a sample from a patient; (b) detecting in the sample an amount of miRNA renal cancer markers; and (c) comparing the detected amount with an amount detected for a standard.

The term “detect” or “detecting” includes assaying, imaging or otherwise establishing the presence or absence of the target renal cancer markers or polynucleotides encoding the markers, subunits thereof, or combinations of reagent bound targets, and the like, or assaying for, imaging, ascertaining, establishing, or otherwise determining one or more factual characteristics of a renal tissue phase or renal disease including cancer, metastasis, stage, or similar conditions. The term encompasses diagnostic, prognostic, and monitoring applications for the renal cancer markers and polynucleotides encoding the markers.

The invention also provides a method of assessing whether a patient is afflicted with or has a pre-disposition for renal disease, in particular renal cancer, the method by comparing: (a) levels of renal cancer markers associated with the renal disease in a sample from the patient; and (b) normal levels of renal cancer markers associated with the renal disease in samples of the same type obtained from control patients not afflicted with the disease, wherein altered levels of the renal cancer markers relative to the corresponding normal levels of such markers is an indication that the patient is afflicted with renal disease.

In one embodiment, the invention also provides a method of assessing whether a patient is afflicted with or has a pre-disposition for renal disease, in particular renal cancer, the method by comparing: (a) levels of protein renal cancer markers and/or the protein renal cancer polynucleotide markers associated with the renal disease in a sample from the patient; and (b) normal levels of renal cancer markers and/or the protein renal cancer polynucleotide markers associated with the renal disease in samples of the same type obtained from control patients not afflicted with the disease, wherein altered levels of the renal cancer markers and/or the protein renal cancer polynucleotide markers relative to the corresponding normal levels of such markers is an indication that the patient is afflicted with renal disease.

A further embodiment of the invention provides a method for assessing whether a patient is afflicted with or has a pre-disposition for renal cancer, where higher levels of protein renal cancer markers, including without limitation tryptophan-5-monooxygenase activation protein (also called YWHAH), Poly (rc)-binding protein 2, and CAPNS1, in a sample relative to the corresponding normal levels is an indication that the patient is afflicted with renal cancer.

Another embodiment of the invention provides a method for assessing whether a patient is afflicted with or has a pre-disposition for renal cancer, where lower levels of protein renal cancer markers, including without limitation prothymosin alpha, in a sample relative to the corresponding normal levels is an indication that the patient is afflicted with renal cancer.

In another embodiment, the invention further provides a method of assessing whether a patient is afflicted with or has a pre-disposition for renal disease, in particular renal cancer, by comparing: (a) levels of miRNA renal cancer markers associated with the renal disease in a sample from the patient; and (b) normal levels of miRNA renal cancer markers associated with the renal disease in samples of the same type obtained from control patients not afflicted with the disease, wherein altered levels of the miRNA renal cancer markers relative to the corresponding normal levels of such markers is an indication that the patient is afflicted with renal disease.

A further embodiment of the invention provides a method for assessing whether a patient is afflicted with or has a pre-disposition for renal cancer, where higher levels of miRNA renal cancer markers, including without limitation mir-34a, miR-155, miR-200c, miR-210, and/or miR-1974, in a sample relative to the corresponding normal levels is an indication that the patient is afflicted with renal cancer.

In a further aspect, the invention provides a method for screening a subject for renal disease by: (a) obtaining a biological sample from a subject; (b) detecting the amount of renal cancer markers associated with the disease in the sample; and (c) comparing the amount of renal cancer markers detected to a predetermined standard, where detection of a level of renal cancer markers that differs significantly from the standard indicates renal disease.

In an embodiment, a significant difference between the levels of renal cancer marker levels in a patient and normal levels is an indication that the patient is afflicted with or has a predisposition to renal disease. In a further embodiment, the level of renal cancer markers is significantly higher compared to the standard and indicative of renal disease. In another embodiment, the level of renal cancer markers is significantly lower compared to the standard and indicative of renal disease. In another embodiment, the renal cancer markers detected are protein renal cancer markers or the protein renal cancer polynucleotide markers. In a further embodiment, the renal cancer markers detected are miRNA renal cancer markers.

In a particular embodiment of the invention, the amount of protein renal cancer marker(s), including without limitation YWHAH and CAPNS1, detected is greater than that of a standard and is indicative of renal disease, in particular renal cancer. In another particular embodiment the amount of renal cancer marker(s), including without limitation prothymosin alpha, detected is lower than that of a standard and is indicative of renal disease, in particular renal cancer.

In aspects of the methods of the invention, the methods are non-invasive for detecting renal disease which in turn allow for diagnosis of a variety of conditions or diseases associated with the kidney.

In particular, the invention provides a non-invasive non-surgical method for detection, diagnosis or prediction of renal disease, including, without limitation, renal cancer and RCC, in a subject, by: (a) obtaining a sample of body fluids, including without limitation blood, plasma, serum, urine or saliva, and/or a tissue sample from the subject; (b) subjecting the sample to a procedure to detect renal cancer markers in the body fluids and/or the tissue sample; (c) detecting, diagnosing, and predicting renal disease by comparing the levels of renal cancer markers to the levels of markers obtained from a control subject with no renal disease.

In an embodiment, renal disease is detected, diagnosed, or predicted by determination of increased levels of protein renal cancer markers, including without limitation one or more of the protein renal cancer markers indicated to be up-regulated in Table 8, when compared to such levels obtained from the control.

In another embodiment, renal disease is detected, diagnosed, or predicted by determination of decreased levels of protein renal cancer markers, including without limitation one or more of the protein renal cancer marked indicated to be down-regulated in Table 8, when compared to such levels obtained from the control.

In an embodiment, renal disease is detected, diagnosed, or predicted by determination of increased levels of miRNA renal cancer markers, including without limitation one or more of the miRNA renal cancer markers indicated to be up-regulated in Table 9 or the miRNA renal cancer markers in Table 12, when compared to such levels obtained from the control.

In another embodiment, renal disease is detected, diagnosed, or predicted by determination of decreased levels of miRNA renal cancer markers, including without limitation one or more of the miRNA renal cancer marked indicated to be down-regulated in Table 9, when compared to such levels obtained from the control.

Another aspect of the invention provides for a method for assessing the aggressiveness or indolence of a renal disease in particular cancer (e.g., staging), by comparing: (a) levels of renal cancer markers with the renal disease in a patient sample; and (b) normal levels of the renal cancer markers in a control sample.

In an embodiment, a significant difference between the levels in the sample and the normal levels is an indication that the renal disease, in particular cancer, is aggressive or indolent. In a particular embodiment, the levels of renal cancer markers are higher than normal levels. In another particular embodiment, the levels of renal cancer markers are lower than normal levels. In another embodiment, the renal cancer markers are protein renal cancer markers or protein renal cancer polynucleotide markers. In a further embodiment, the renal cancer markers are miRNA renal cancer markers.

In an embodiment, aggressiveness or indolence of a renal disease assessed by determination of increased levels of protein renal cancer markers, including without limitation one or more of the protein renal cancer markers indicated to be up-regulated in Table 8, when compared to such levels obtained from the control.

In another embodiment, aggressiveness or indolence of a renal disease assessed by determination of decreased levels of protein renal cancer markers, including without limitation one or more of the protein renal cancer marked indicated to be down-regulated in Table 8, when compared to such levels obtained from the control.

In an embodiment, aggressiveness or indolence of a renal disease assessed by determination of increased levels of miRNA renal cancer markers, including without limitation one or more of the miRNA renal cancer markers indicated to be up-regulated in Table 9 or the miRNA renal cancer markers in Table 12, when compared to such levels obtained from the control.

In another embodiment, aggressiveness or indolence of a renal disease assessed by determination of decreased levels of miRNA renal cancer markers, including without limitation one or more of the miRNA renal cancer marked indicated to be down-regulated in Table 9, when compared to such levels obtained from the control.

In an embodiment, the invention provides a method for diagnosing and/or monitoring RCC by comparing: (a) levels of YWHAH or polynucleotides encoding YWHAH in a sample from the patient; and (b) normal levels of YWHAH or polynucleotides encoding the same in samples of the same type obtained from control patients not afflicted with renal cancer or having a different stage of renal cancer, wherein altered levels of YWHAH or polynucleotides encoding same compared with the corresponding normal levels is an indication that the patient is afflicted with RCC.

In another embodiment, a method is provided for diagnosing and/or monitoring RCC by comparing: (a) levels of CAPNS1 or polynucleotides encoding CAPNS1 in a sample from the patient; and (b) normal levels of CAPNS1 or polynucleotides encoding same in samples of the same type obtained from control patients not afflicted with renal cancer or having a different stage of renal cancer, wherein altered levels of CAPNS1 or polynucleotides encoding same compared with the corresponding normal levels is an indication that the patient is afflicted with RCC.

In an embodiment, a method is provided for diagnosing and/or monitoring RCC by comparing: (a) levels of SERPING1 or polynucleotides encoding SERPING1 in a sample from the patient; and (b) normal levels of SERPING1 or polynucleotides encoding same in samples of the same type obtained from control patients not afflicted with renal cancer or having a different stage of renal cancer, wherein altered levels of SERPING1 or polynucleotides encoding same compared with the corresponding normal levels is an indication that the patient is afflicted with RCC.

In an aspect, the invention provides a method for determining whether a cancer has metastasized or is likely to metastasize in the future, by comparing: (b) levels of renal cancer markers in a patient sample; and (b) normal levels (or non-metastatic levels) of the renal cancer markers in a control sample.

In an embodiment, a significant difference between the levels in the patient sample and the normal levels is an indication that the cancer has metastasized or is likely to metastasize in the future.

In another aspect, the invention provides a method for monitoring the progression of renal disease, in particular renal cancer in a patient, by: (a) detecting renal cancer markers or polynucleotides encoding the markers associated with the disease in a sample from the patient at a first time point; (b) repeating step (a) at a subsequent point in time; and (c) comparing the levels detected in (a) and (b), and therefrom monitoring the progression of the renal disease.

The invention further contemplates a method for determining the effect of an environmental factor on the renal tissue, or renal disease, by comparing renal cancer renal cancer markers in the presence and absence of the environmental factor. One embodiment of such method uses protein renal cancer marker or protein renal cancer polynucleotide markers. A further embodiment of the invention uses miRNA renal cancer markers.

The invention also provides a method for assessing the potential efficacy of a test agent for inhibiting renal disease, and a method of selecting an agent for inhibiting renal disease. In another aspect, the invention provides for a method for assessing the carcinogenic potential of a test compound.

The invention contemplates a method of assessing the potential of a test compound to contribute to a renal disease by: (a) maintaining separate aliquots of renal diseased cells in the presence and absence of the test compound; and (b) comparing the levels of renal cancer markers associated with the disease in each of the aliquots.

A significant difference between the levels of renal cancer markers or polynucleotides encoding the markers in an aliquot maintained in the presence of (or exposed to) the test compound relative to the aliquot maintained in the absence of the test compound, indicates that the test compound potentially contributes to renal disease. In one embodiment, the renal cancer markers are protein renal cancer markers or protein renal cancer polynucleotide markers. In another embodiment, the renal cancer markers are miRNA renal cancer markers.

The invention further relates to a method of assessing the efficacy of a therapy for inhibiting renal disease in a patient by comparing: (a) levels of renal cancer markers associated with disease in a first sample from the patient obtained from the patient prior to providing at least a portion of the therapy to the patient; and (b) levels of renal cancer markers associated with disease in a second sample obtained from the patient following therapy.

In an embodiment, a significant difference between the levels of renal cancer markers in the second sample relative to the first sample is an indication that the therapy is efficacious for inhibiting renal disease. In another embodiment, the renal cancer markers are protein renal cancer markers or protein renal cancer polynucleotide markers. In a further embodiment, the renal cancer markers are miRNA renal cancer markers.

In a particular embodiment, the method is used to assess the efficacy of a therapy for inhibiting renal disease, including without limitation renal cancer or RCC, where lower levels of renal cancer markers in the second sample relative to the first sample, is an indication that the therapy is efficacious for inhibiting the disease. In another embodiment, the renal cancer markers are protein renal cancer markers or protein renal cancer polynucleotide markers. In a further embodiment, the renal cancer markers are miRNA renal cancer markers.

The “therapy” may be any therapy for treating renal disease, in particular renal cancer, including but not limited to therapeutics, radiation, immunotherapy, gene therapy, and surgical removal of tissue. Therefore, the method can be used to evaluate a patient before, during, and after therapy.

Certain methods of the invention employ binding agents that specifically recognize renal cancer markers.

In another aspect, the invention provides methods for determining the presence or absence of renal disease, in particular renal cancer, in a patient, by: (a) contacting a biological sample obtained from a patient with one or more binding agent that specifically binds to one or more renal cancer markers associated with the disease; and (b) detecting in the sample an amount of marker that binds to the binding agent, relative to a predetermined standard or cut-off value, and therefrom determine the presence or absence of renal disease in the patient from such results.

In a further aspect, the invention relates to a method for diagnosing and monitoring a renal disease, in particular renal cancer, in a subject by quantifying one or more renal cancer markers associated with the disease in a biological sample from the subject, by: (a) reacting the biological sample with one or more binding agent specific for the renal cancer markers that are directly or indirectly labelled with a detectable substance; and (b) detecting the detectable substance. In one embodiment, antibodies are used as binding agents to recognize the protein renal cancer markers. In a further embodiment, polynucleotides are used as binding agents to recognize the protein renal cancer polynucleotide markers. In another embodiment, polynucleotides are used as binding agents to recognize the miRNA renal cancer markers.

In another aspect the invention provides a method of using an antibody to detect expression of one or more protein renal cancer marker in a sample, by: (a) combining antibodies specific for one or more protein renal cancer marker with a sample under conditions which allow the formation of antibody-protein marker complexes; and (b) detecting complex formation, wherein complex formation indicates expression of the protein renal cancer marker in the sample wherein expression may be compared with standards and is diagnostic of a renal disease, in particular renal cancer.

Embodiments of the invention may also involve: (a) reacting a biological sample from a subject with antibodies specific for one or more renal cancer markers which are directly or indirectly labelled with an enzyme; (b) adding a substrate for the enzyme wherein the substrate is selected so that the substrate, or a reaction product of the enzyme and substrate forms fluorescent complexes; (c) quantitating one or more renal cancer markers in the sample by measuring fluorescence of the fluorescent complexes; and (d) comparing the quantitated levels to levels obtained for other samples from the subject patient, or control subjects.

In another embodiment the quantitated levels are compared to levels quantitated for control subjects, such as normal or benign tumours, without a renal disease, particularly renal cancer, wherein an increase in renal cancer marker levels compared with the control subjects is indicative of renal disease.

In a further embodiment the quantitated levels are compared to levels quantitated for control subjects, such as normal or benign tumours, without a renal disease, particularly renal cancer, wherein a decrease in renal marker levels compared with the control subjects is indicative of renal disease.

In another aspect, the invention provides a method by: (a) incubating a biological sample with first antibodies specific for one or more renal cancer markers which are directly or indirectly labelled with a detectable substance, and second antibodies specific for one or more renal cancer markers which are immobilized; (b) detecting the detectable substance thereby quantitating renal cancer markers in the biological sample; and (c) comparing the quantitated renal cancer markers with levels for a pre-determined standard.

The standard may correspond to levels quantitated for samples from control subjects without renal cancer (normal or benign), with a different disease stage, or from other samples of the subject. In an embodiment, increased levels of renal cancer markers as compared to the standard may be indicative of renal cancer. In another embodiment, lower levels of renal cancer markers as compared to a standard may be indicative of renal cancer.

Protein renal cancer marker levels can be determined by constructing an antibody microarray in which binding sites include immobilized, preferably monoclonal, antibodies specific to a substantial fraction of marker-derived protein renal cancer markers of interest.

Other methods of the invention employ one or more polynucleotides capable of hybridizing to one or more protein renal cancer polynucleotide markers or miRNA renal cancer markers. Further methods of the invention employ one or more polynucleotides capable of hybridizing to one or more miRNA renal cancer markers. Thus, methods can be used to monitor a renal disease, in particular renal cancer, by detecting protein renal cancer polynucleotide markers or the miRNA renal cancer markers associated with the disease.

Thus, the present invention relates to a method for diagnosing and monitoring a renal disease, including without limitation renal cancer, RCC or related condition, in a sample from a subject, by: (a) isolating nucleic acids, preferably mRNA or miRNA, from the sample; and (b) detecting protein renal cancer polynucleotide markers associated with renal diseases or the miRNA renal cancer markers in the sample, wherein the presence of different levels of protein renal cancer polynucleotide markers or the miRNA renal cancer markers in the sample compared to a standard or control may be indicative of renal disease, disease stage, and/or a negative or positive prognosis, such as longer progression-free and overall survival.

In embodiments of the invention, protein renal cancer polynucleotide marker positive-tumours (e.g., higher levels of the polynucleotides compared to a control normal or benign sample) or miRNA renal cancer marker positive-tumours (e.g., higher levels of the miRNA renal cancer marker compared to a control normal or benign sample) are a negative diagnostic indicator. Positive tumors can be indicative of renal cancer, advanced stage disease, lower progression-free survival, and/or overall survival.

In embodiments of the invention, protein renal cancer polynucleotide marker negative-tumours (e.g., lower levels of the polynucleotides compared to a control normal or benign sample) or miRNA renal cancer marker negative-tumours (e.g., lower levels of the miRNA renal cancer marker compared to a control normal or benign sample) are a negative diagnostic indicator. Negative tumors can be indicative of renal cancer, advanced stage disease, lower progression-free survival, and/or overall survival.

The invention provides methods for determining the presence or absence of a renal disease in a subject, by: (a) detecting in the sample levels of nucleic acids that hybridize to one or more miRNA renal cancer markers or polynucleotide renal cancer markers associated with the disease; (b) comparing the levels with a pre-determined standard or cut-off value; and (c) determining the presence or absence of renal disease in the subject.

In an embodiment, the invention provides methods for determining the presence or absence of renal disease, such as RCC or another renal cancer, in a subject, by: (a) contacting a sample obtained from the subject with oligonucleotides that hybridize to one or more miRNA renal cancer markers or polynucleotides encoding renal cancer markers; (b) detecting in the sample a level of nucleic acids that hybridize to the polynucleotides relative to a predetermined cut-off value; and (c) determining the presence or absence of renal cancer in the subject.

Within certain embodiments, the amount of polynucleotides that are mRNA or miRNA are detected via polymerase chain reaction using, for example, oligonucleotide primers that hybridize to one or more protein renal cancer polynucleotide markers or miRNA renal cancer markers, or complements of such polynucleotides. Within other embodiments, the amount of mRNA or miRNA is detected using a hybridization technique, employing oligonucleotide probes that hybridize to one or more protein renal cancer polynucleotide markers or miRNA renal cancer markers, or complements of such polynucleotides.

When using mRNA or miRNA detection, the method may be carried out by combining isolated mRNA or miRNA with reagents to convert to cDNA according to standard methods well known in the art, treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of one or more of the protein renal cancer polynucleotide markers or miRNA renal cancer marker in the sample. For mRNA or miRNA, the analyzing step may be accomplished using Northern Blot analysis to detect the presence of protein renal cancer polynucleotide markers or miRNA renal cancer marker in the sample. The analysis step may be further accomplished by quantitatively detecting the presence of protein renal cancer polynucleotide markers or miRNA renal cancer marker in the amplification product, and comparing the quantity of marker detected against a panel of expected values for the known presence or absence of such markers in normal and malignant tissue derived using similar primers.

Therefore, the invention provides a method wherein mRNA or miRNA is detected by: (a) isolating mRNA or miRNA from a sample and combining the mRNA or miRNA with reagents to convert it to cDNA; (b) treating the converted cDNA with amplification reaction reagents and nucleic acid primers that hybridize to one or more of the protein renal cancer polynucleotide markers or miRNA renal cancer marker to produce amplification products; (c) analyzing the amplification products for determining the amount of mRNA present encoding the protein renal cancer marker or the amount of miRNA renal cancer marker present; and (d) comparing the determined amount of mRNA or miRNA to an amount detected against a panel of expected values for normal and diseased tissue (e.g., malignant tissue) derived using similar methods.

In particular embodiments of the invention, RT-PCR can be used to amplify the mRNA for protein renal cancer markers or miRNA renal cancer marker for detection and analysis. Other embodiments of the invention use quantitative RT-PCR to quantitatively determine amount of mRNA for protein renal cancer markers or miRNA renal cancer marker. Further embodiments of the invention use real time RT-PCR for quantification and analysis.

In particular embodiments of the invention, the methods described herein utilize the protein renal cancer polynucleotide markers and/or miRNA renal cancer marker placed on a microarray so that the expression status of each of the markers may be assessed.

In a particular aspect, the invention provides a microarray including a defined set of genes encoding for protein renal cancer markers (i.e., at least 2, 3, 4, or 5 genes listed in Table 2 or Table 4 or Table 5 or Table 6 or Table 6 or Table 8) or a defined set of miRNA renal cancer markers (i.e., at least 2, 3, 4, or 5 miRNA listed in Table 12) whose expression is significantly altered by renal disease. The invention further relates to the use of the microarray as a prognostic tool to predict or diagnose renal disease. In an embodiment, the renal microarray discriminates between renal diseases resulting from different etiologies.

In an embodiment, the invention provides for oligonucleotide arrays including marker sets described herein. The microarrays provided by the present invention may include probes to markers able to distinguish renal disease. In particular, the invention provides oligonucleotide arrays including probes to a subset or subsets of at least 5 to 10 protein renal cancer polynucleotide markers or a subset or subsets of at least 5 to 10 miRNA renal cancer marker markers, up to a full set of markers which distinguish renal disease.

The invention also contemplates a method by administering to cells or tissues imaging agents that carry labels for imaging and bind to renal cancer markers and optionally other markers of a renal disease, and then imaging the cells or tissues.

In an aspect the invention provides an in vivo method by administering to a subject an agent that has been constructed to target one or more renal cancer markers.

In a particular embodiment, the invention contemplates an in vivo method by administering to a mammal one or more agent that carries a label for imaging and binds to one or more renal cancer markers, and then imaging the mammal.

In another aspect, the invention provides an in vivo method for imaging renal cancer, by: (a) injecting a patient with an agent that binds to one or more renal cancer markers, the agent carrying a label for imaging the renal cancer; (b) allowing the agent to incubate in vivo and bind to one or more renal cancer markers associated with the renal cancer; and (c) detecting the presence of the label localized to the cancer. In one embodiment, the agent is an antibody that recognizes a protein renal cancer marker. In another embodiment, the agent is a chemical entity that recognizes a renal cancer marker.

An agent carries a label to image a renal marker and optionally other markers. Examples of labels useful for imaging are radiolabels, fluorescent labels (e.g., fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed.

The invention also contemplates the localization or imaging methods described herein using multiple markers for a renal disease, including without limitation renal cancer, RCC or related conditions.

The invention also relates to kits for carrying out the methods of the invention. In an embodiment, a kit is for assessing whether a patient is afflicted with a renal disease, including without limitation renal cancer, RCC or related conditions, and the kit includes reagents for assessing one or more renal cancer markers.

The invention further provides kits including marker sets described herein. In an aspect of the invention, the kit contains a microarray ready for hybridization to target renal cancer polynucleotide markers and the software needed for the data analysis.

The invention also provides a diagnostic composition including protein renal cancer markers, miRNA renal cancer markers or polynucleotide encoding the markers. A composition is also provided including a probe that specifically hybridizes to protein renal cancer polynucleotide markers or miRNA renal cancer markers, or a fragment thereof, or an antibody specific for protein renal cancer markers or a fragment thereof. In another aspect, a composition is provided including protein renal cancer polynucleotide markers or miRNA renal cancer marker specific primer pairs capable of amplifying such polynucleotides using polymerase chain reaction methodologies. The probes, primers or antibodies can be labeled with a detectable substance.

Still further the invention relates to therapeutic applications for renal diseases, in particular renal cancer, employing protein renal cancer markers, miRNA renal cancer markers, and polynucleotide encoding the markers, and/or binding agents for such markers.

In an aspect, the invention relates to compositions including markers or parts thereof associated with a renal disease, or antibodies specific for protein renal cancer markers associated with a renal disease, and a pharmaceutically acceptable carrier, excipient, or diluent. Another of the invention provides a method for treating or preventing a renal disease, in particular renal cancer, in a patient, by administering to a patient in need thereof, markers or parts thereof associated with renal disease, antibodies specific for protein renal cancer markers, or a composition of the invention. In an aspect the invention provides a method of treating a patient afflicted with or at risk of developing a renal disease (e.g., renal cancer) by inhibiting expression of protein renal cancer polynucleotide markers or miRNA renal cancer markers.

An embodiment of the invention includes antisense oligonucleotides complementary to protein renal cancer polynucleotide markers or miRNA renal cancer markers delivered to diseased cells for regulation of gene expression.

In an aspect, the invention provides antibodies specific for protein renal cancer markers associated with a disease, such as RCC, that can be used therapeutically to destroy or inhibit the disease, such as growth of marker expressing cancer cells), or to block marker activity associated with a disease. In an aspect, the renal cancer markers may be used in various immunotherapeutic methods to promote immune-mediated destruction or growth inhibition of tumors expressing the renal cancer markers.

The invention also contemplates a method of using renal cancer markers or parts thereof, or antibodies specific for the protein renal cancer markers in the preparation or manufacture of a medicament for the prevention or treatment of a renal disease, including without limitation renal cancer, RCC or related conditions.

Another aspect of the invention relates to the use of protein renal cancer markers, peptides derived therefrom, or chemically produced (synthetic) peptides, or any combination of these molecules, for use in the preparation of vaccines to prevent a renal disease and/or to treat a renal disease.

The invention contemplates vaccines for stimulating or enhancing in a subject to whom the vaccine is administered, production of antibodies directed against one or more renal cancer markers.

The invention also provides a method for stimulating or enhancing in a subject production of antibodies directed against one or more renal cancer markers by administering to the subject a vaccine of the invention in a dose effective for stimulating or enhancing production of the antibodies.

The invention further provides a method for treating, preventing, or delaying recurrence of a renal disease, including without limitation renal cancer, RCC or related conditions by administering to the subject a vaccine of the invention in a dose effective for treating, preventing, or delaying recurrence of a renal disease including without limitation renal cancer, RCC or related conditions.

The invention contemplates the methods, compositions, and kits described herein using additional markers associated with a renal disease, including without limitation renal cancer, RCC or related conditions. The methods described herein may be modified by including reagents to detect the additional markers, or polynucleotides for the markers.

In particular, the invention contemplates the methods described herein using multiple markers for renal cancer, such as RCC. Therefore, the invention contemplates a method for analyzing a biological sample for the presence of renal cancer markers and other markers that are specific indicators of cancer, in particular renal cancer. The methods described herein may be modified by including reagents to detect the additional markers, or nucleic acids for the additional markers.

In embodiments of the invention, the methods, compositions, and kits use a panel of the protein renal cancer markers listed in Table 8 and/or the miRNA renal cancer markers listed in Table 12 or a precursor thereof.

In embodiments of the invention, biological samples may be obtained from tissues, extracts, cell cultures, cell lysates, lavage fluid, or physiological fluids. In further embodiments of the invention, the sample is a renal tumour tissue.

Table 1 shows the distribution of over- and underexpressed proteins in RCC specimens in the two runs. There were 65 proteins that were recognized in both runs as being underexpressed, while 34 proteins as being overexpressed. There was a statistically significant positive correlation of normalized protein levels identified in both runs (rp=0.695, p<0.001) (FIG. 2). Cellular localization of the inventors' extracted proteins was determined using the GO analysis and most proteins were assigned to cellular compartments: 34% of the proteins were cytoplasmic, 13% nuclear, 11% were localized to the mitochondria, 4% membranous, and 6% were extracellular proteins (FIG. 3A). A comparable distribution was seen for the overexpressed proteins (FIG. 3B). Proteins of cytoplasmic and membranous distribution are of particular importance, as they may have potential for use as tumor markers in biological fluids. Table 8 shows a partial list of the top dysregulated proteins in RCC compared to their normal counterparts, along with their chromosomal locations: 49 proteins demonstrated an average increase of ≧2.0 fold; nine proteins showed ≧3.0 fold average increase in expression levels. Underexpressed proteins ranged from 0.04-0.67 in expression relative to normal tissues.

In order to examine if these dysregulations were specific to RCC, or represented a non-specific response by the kidney cells, the inventors analyzed two control specimens simultaneously: a transitional cell carcinoma (TCC, with a distinct origin from the transitional urothelium of the kidney) and kidney tissue from a case of end-stage glomerulonephritis. Protein expressions were normalized using expression levels in normal kidney tissue, and expressed as fold changes of normal. The normalized average expression in RCC was lower than normalized non-cancerous renal failure in 454 proteins and higher in 483 proteins (p=0.020) (FIG. 4A). RCC normalized average expression values were also lower than those of normalized TCC in 415 proteins, and higher in 522 proteins (p=0.005) (FIG. 4B). Table 3 shows the relative normalized protein expression ratios between RCC, renal failure and TCC. The inventors also compared the inventors' results with four previously published reports of differential protein expression in kidney cancer (Craven et al. 2006; Perego et al. 2005; Sarto et al. 1997; Shi et al. 2004). There are 24 proteins from the inventors' list of proteins that were identified in the other reports; 15 of them were shown to be differentially expressed by one report, five identified by two additional reports, and four recognized by three studies (Table 4).

The inventors performed an in silico validation of the inventors' results by different approaches. In addition, the inventors did a thorough literature search for individual up- and downregulated proteins: a few of the inventors' dysregulated proteins were found to be previously reported as tumor markers for RCC (vide infra). The inventors also performed a database search through the SwissProt Knowledgebase. Table 5 shows a partial list of proteins from the inventors' list with documented expression in the kidney.

To validate the inventors' protein findings and examine whether they are reflected at the mRNA level, the inventors performed SAGE and EST analysis. SAGE data verified overexpression of 65% of all genes with informative expression data (data not shown). When focusing on proteins that were identified in both runs, the SAGE mRNA data were in concordance with the protein results in 84% of cases (Table 6). EST analysis of informative genes using both the EST ProfileViewer and the Digital Gene Expression Displayer search engines confirmed upregulation in cancer compared to normal in 74% of cases by at least one search engine (Table 7).

Immuohistochemistry and dot blot were employed to further validate the protein expression results. Vimentin, which was found to overexpressed in RCC in the inventors'MS/MS analyses, was validated by immunohistochemistry (FIG. 5). Staining for vimentin antibodies was weak or largely absent in normal cancer tissue while significant staining can be observed in RCC tissue. Similar result was obtained using phospho-S6 ribosomal protein (another overexpressed protein) antibodies (data not shown). Three highly overexpressed proteins were selected for dot blot analysis. (FIG. 6). Dot blot analysis showed all 3 proteins were overexpressed in RCC tissue, further confirming the MS/MS results.

In order to validate the potential clinical utility of the dysregulated proteins as potential serum biomarkers, the inventors performed a literature and bioinformatics search (through public databases) of the inventors' upregulated proteins and were able to identify 23 of these proteins being secreted in the blood (data not shown). The inventors are currently performing individual validation of few of these biomarkers in serum of RCC patients.

MicroRNAs (miRNAs) are small stretches of non-protein coding RNA. They have been shown to be differentially expressed in many malignancies, including breast, lung, prostate and ovarian cancers. In this study, the inventors performed a miRNA microarray analysis to identify the differentially expressed miRNAs in the clear cell subtype of kidney cancer, a common urologic malignancy.

A total of 80 differentially expressed miRNAs were identified in clear cell renal cell carcinoma (ccRCC). Bioinformatics and literature searches showed that many of these have been reported to be dysregulated in other malignancies and have a potential role in cancer pathogenesis. The inventors validated the top dysregulated miRNAs by quantitative RT-PCR analysis. The differentially expressed miRNAs showed a significant correlation with reported regions of chromosomal aberration sites that included regions of amplification or loss. The inventors also utilized bioinformatics-based target prediction algorithms to identify potential gene targets of these dysregulated miRNAs. Preliminary analyses showed that some of these targets can be directly involved in RCC pathogenesis. A subgroup of the dysregulated miRNAs is previously reported to be hypoxia-induced. This implies a direct role in kidney cancer pathogenesis, since hypoxia is documented to have a central role in RCC pathogenesis.

The inventors, through microarray and real-time PCR analysis, identified miRNA that are dysregulated in clear-cell RCC (“ccRCC”) and the potential involvement of these miRNAs and their targets in the RCC pathogenesis and/or progression. There is also significant correlation between dysregulated miRNAs and chromosomal aberrations in ccRCC.

Accordingly, the inventors describe herein methods for detecting the presence of a renal disease, including without limitation renal cancer, RCC or similar conditions, in a sample, the absence of a disease in a sample, the stage or grade of the disease, and other characteristics of renal diseases that are relevant to prevention, diagnosis, characterization, and therapy of renal diseases such as renal cell carcinoma in a patient, for example, the metastatic potential of a renal cell carcinoma assessing the histological type of neoplasm associated with a renal cancer, the indolence or aggressiveness of a renal cell carcinoma, and other characteristics of renal diseases that are relevant to prevention, diagnosis, characterization, and therapy of renal diseases such as renal cell carcinoma in patient. Methods are also provided for assessing the efficacy of one or more test agents for inhibiting a renal disease, assessing the efficacy of a therapy for a renal diseases such as renal cell carcinoma, monitoring the progression of a renal diseases such as renal cell carcinoma, selecting an agent or therapy for inhibiting a renal diseases such as renal cell carcinoma, treating a patient afflicted with a renal cell carcinoma, inhibiting a renal diseases such as renal cell carcinoma, and assessing the disease (e.g., carcinogenic) potential of a test compound.

Abbreviation Index. For convenience, certain abbreviations used in the description, tables, figures, and appended claims are defined here: iTRAQ, isobaric tags for relative and absolute quantification; LC, liquid chromatography; MS/MS, tandem mass spectrometry; PCM, potential cancer marker; RCC, Renal cell carcinoma; LCM, laser capture microdissection; PBS, phosphate-buffered saline; SCX, strong cation exchange; ID, internal diameter; RP, reverse phase; IDA, information-dependent acquisition; TBS, tris-buffered saline; AUC, area under the curve; RSD, relative standard deviation; TMA, tissue microarray.

Glossary. For convenience, certain terms employed in the specification, examples, and appended claims are collected here.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” Furthermore, it is to be understood that “a”, “an,” and “the” include plural referents unless the content clearly dictates otherwise. Thus, for example, reference to a composition or method that includes “a renal cancer marker” will encompass two or renal cancer markers. The term “about” means plus or minus 0.1 to 50%, 5-50%, or 10-40%, preferably 10-20%, more preferably 10% or 15%, of the number to which reference is being made.

“Renal disease” refers to any disorder, disease, condition, syndrome or combination of manifestations or symptoms recognized or diagnosed as a disorder of kidney, including but not limited to inflammation and cancer precursors or carcinoma.

“Renal disease” includes malignant renal carcinoma including but not limited to RCC, clear-cell RCC, and transitional cell carcinomas.

The terms “sample”, “biological sample”, and the like mean a material known or suspected of expressing or containing one or more renal cell carcinoma polynucleotide markers. A test sample can be used directly as obtained from the source or following a pretreatment to modify the character of the sample. The sample can be derived from any biological source, such as tissues, extracts, or cell cultures, including cells (e.g., tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid, lavage fluid, and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like.

In embodiments of the invention the sample is a mammalian tissue sample. In a particular embodiment, the tissue is kidney tissue. In further embodiments, the sample is a renal tumour tissue.

In another embodiment the sample is a human physiological fluid. In a particular embodiment, the sample is human serum.

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). The target polynucleotides can include RNA, including, without limitation, total cellular RNA, miRNA, poly(A)+ messenger RNA (mRNA) or fraction thereof, cytoplasmic mRNA, or RNA transcribed from cDNA (i.e., cRNA; see, for example, Linsley & Schelter, patent application Ser. No. 09/411,074, or U.S. Pat. Nos. 5,545,522; 5,891,636; or 5,716,785). Methods for preparing total and poly(A)+ RNA are well known in the art, and are described generally, for example, in Sambrook et al., (1989, Molecular Cloning—A Laboratory Manual (2nd Ed.), Vols. 1-3, Cold Spring Harbor Laboratory, Cold Spring Harbor, N.Y.) and Ausubel et al., eds. (1994, Current Protocols in Molecular Biology, Vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a non-ionic detergent, followed by microcentrifugation to remove the nuclei and hence the bulk of the cellular DNA. (See Chirgwin et al., 1979, Biochemistry 18:5294-5299). Poly(A)+RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

It may be desirable to enrich mRNA with respect to other cellular RNAs, such as transfer RNA (tRNA) and ribosomal RNA (rRNA). Most mRNAs contain a poly(A) tail at their 3′ end allowing them to be enriched by affinity chromatography, for example, using oligo(dT) or poly(U) coupled to a solid support, such as cellulose or Sephadex™ (see Ausubel et al., eds., 1994, Current Protocols in Molecular Biology, Vol. 2, Current Protocols Publishing, New York). Bound poly(A)+mRNA is eluted from the affinity column using 2 mM EDTA/0.1% SDS.

A sample of RNA can include a plurality of different RNA molecules each with a different nucleotide sequence. In an aspect of the invention, the RNA molecules contain mRNA molecules. In a further aspect of the invention, the mRNA molecules in the RNA sample include at least 100 different nucleotide sequences. In another aspect of the invention, the RNA molecules contain miRNA molecules or its precursors thereof.

Target polynucleotides can be detectably labelled at one or more nucleotides using methods known in the art. The label is preferably uniformly incorporated along the length of the RNA, and more preferably, is carried out at a high degree of efficiency. The detectable label can be a luminescent label, fluorescent label, bio-luminescent label, chemiluminescent label, radiolabel, and colorimetric label. In a particular embodiment, the label is a fluorescent label, such as a fluorescein, a phosphor, a rhodamine, or a polymethine dye derivative. Commercially available fluorescent labels include, for example, fluorescent phosphoramidites, such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.).

Target polynucleotides from a patient sample can be labelled differentially from polynucleotides of a standard. The standard can include target polynucleotides from normal individuals (i.e., those not afflicted with or pre-disposed to renal disease), in particular pooled from samples from normal individuals. The target polynucleotides can be derived from the same individual, but taken at different time points, and thus indicate the efficacy of a treatment by a change in expression of the markers, or lack thereof, during and after the course of treatment.

The terms “subject”, “individual”, and “patient” refer to a warm-blooded animal such as a mammal. In particular, the terms refer to a human. A subject, individual or patient may be afflicted with or suspected of having or being pre-disposed to renal disease or a condition as described herein. The terms also includes domestic animals bred for food or as pets, including horses, cows, sheep, poultry, fish, pigs, cats, dogs, and zoo animals.

Methods herein for administering an agent or composition to subjects/individuals/patients contemplate treatment as well as prophylactic use. Typical subjects for treatment include persons susceptible to, suffering from or that have suffered a condition or disease described herein. In particular, suitable subjects for treatment in accordance with the invention are persons that are susceptible to, suffering from or that have suffered renal cancer.

The term “renal cancer marker” refers to a marker associated with normal or diseased kidney tissue and includes or consists of one or more of the polypeptides that are up-regulated in cancer samples as compared to normal samples in Table 8 or of miRNA that are up-regulated in cancer samples as compared normals samples in Table 12. The term includes native-sequence polypeptides or nucleotides, isoforms, chimeric polypeptides, complexes, all homologs, fragments, precursors, and modified forms and derivatives of the markers, as applicable.

A renal cancer marker may be associated with a renal disease, in particular it may be a renal cancer marker. The term “renal cancer marker” includes a marker associated with protein renal cancer marker listed in Table 8, polynucleotide encoding for renal cancer markers or a miRNA renal cancer marker listed in Table 12 or a precursor thereof.

A “native-sequence polypeptide” includes a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g., alternatively spliced forms or splice variants), and naturally occurring allelic variants.

The expression “polypeptide variant” refers to a polypeptide having at least about 45%, 50%, 55%, 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95% amino acid sequence identity with a native-sequence polypeptide. Particular polypeptide variants have at least 70-80%, 85%, 90%, 95% amino acid sequence identity to the sequences identified in Table 8. Such variants include, for instance, polypeptides wherein one or more amino acid residues are added to, or deleted from, the N- or C-terminus of the full-length or mature sequences of the polypeptide, including variants from other species, but exclude a native-sequence polypeptide. In aspects of the invention variants retain the immunogenic activity of the corresponding native-sequence polypeptide.

Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GIG program package (Devereux J. et al., Nucleic Acids Research 12(1): 387, 1984); BLASTP, BLASTN, and FASTA (Altschul, S. F. et al., J. Molec. Biol. 215: 403-410, 1990). The BLAST X program is publicly available from NCBI and other sources (BLAST Manual, Altschul, S. et al., NCBI NLM NIH Bethesda, Md. 20894; Altschul, S. et al., J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly-available computer programs.

An allelic variant may also be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g., Lys, Arg, His), acidic side chains (e.g., Asp, Glu), uncharged polar side chains (e.g., Gly, Asp, Glu, Ser, Thr, Tyr, and Cys), non-polar side chains (e.g., Ala, Val, Leu, Iso, Pro, Trp), beta-branched side chains (e.g., Thr, Val, Iso), and aromatic side chains (e.g., Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides and amino acid sequences sufficiently identical to or derived from the amino acid sequence of a native polypeptide which includes fewer amino acids than the fulllength polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally-occurring allelic variant may contain conservative amino acid substitutions from the native polypeptide sequence or it may contain a substitution of an amino acid from a corresponding position in a polypeptide homolog, for example, a murine polypeptide.

A renal cancer marker may be part of a chimeric or fusion protein. A “chimeric protein” or “fusion protein” includes all or part (preferably biologically active) of a renal cancer marker operably linked to a heterologous polypeptide (i.e., a polypeptide other than a renal diseases marker). Within the fusion protein, the term “operably linked” is intended to indicate that a renal cancer marker and the heterologous polypeptide are fused in-frame to each other. The heterologous polypeptide can be fused to the N-terminus or C-terminus of a renal cancer marker. A useful fusion protein is a GST fusion protein in which a renal cancer marker is fused to the C-terminus of GST sequences. Another example of a fusion protein is an immunoglobulin fusion protein in which all or part of a renal cancer marker is fused to sequences derived from a member of the immunoglobulin protein family. Chimeric and fusion proteins can be produced by standard recombinant DNA techniques.

A modified form of a polypeptide referenced herein includes modified forms of the polypeptides and derivatives of the polypeptides, including post-translationally modified forms such as glycosylated, phosphorylated, acetylated, methylated or lapidated forms of the polypeptides. For example, an N-terminal methionine may be cleaved from a polypeptide, and a new N-terminal residue may or may not be acetylated. In particular, for chaperonin 10 the first residue, methionine, can be cleaved and the second first residue, alanine can be N-acetylated.

A renal cancer marker may be prepared by recombinant or synthetic methods, or isolated from a variety of sources, or by any combination of these and similar techniques.

“Protein renal cancer polynucleotide marker(s)”, “polynucleotides encoding the protein marker(s)”, and “polynucleotides encoding a protein renal cancer marker” refer to polynucleotides that encode a protein renal cancer markers including native-sequence polypeptides, polypeptide variants including a portion of a polypeptide, an isoform, precursor, complex, a chimeric polypeptide, or modified forms and derivatives of the polypeptides. A renal cancer polynucleotide marker includes or consists of one or more of the polynucleotides encoding the polypeptides listed in Table 8. “miRNA renal cancer markers” include native-sequence polynucleotide, polynucleotide encoding for the miRNA renal cancer marker, polynucleotide variants including a portion of a polynucleotide, an isoform, precursor, complex, a chimeric polynucleotide, or modified forms and derivatives of the polynucleotides. A miRNA renal cancer marker includes or consists of one or more of the miRNA listed in Table 12 or a precursor thereof. “Polynucleotide encoding the markers” include “protein renal cancer polynucleotide marker(s)”, “polynucleotides encoding the protein marker(s)”, “polynucleotides encoding a protein renal cancer marker”.

A renal cancer polynucleotide markers and miRNA renal cancer markers include complementary nucleic acid sequences, and nucleic acids that are substantially identical to these sequences (e.g., having at least about 45%, 50%, 55%, 60%, 65%, 70%, 75%, 80%, 85%, 90%, 95%, 97%, 98%, or 99% sequence identity).

Protein renal cancer polynucleotide markers or miRNA renal cancer markers also include sequences that differ from a native sequence due to degeneracy in the genetic code. As one example, DNA sequence polymorphisms within the nucleotide sequence of a renal cancer marker may result in silent mutations that do not affect the amino acid or nucleotide sequence. Variations in one or more nucleotides may exist among individuals within a population due to natural allelic variation. DNA sequence polymorphisms may also occur which lead to changes in the amino acid sequence of a polypeptide or the nucleotide sequence of a polynucleotide.

Protein renal cancer polynucleotide markers or miRNA renal cancer markers also include nucleic acids that hybridize under stringent conditions, preferably high stringency conditions to renal cancer polynucleotide marker and miRNA renal cancer markers. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

Protein renal cancer polynucleotide markers or miRNA renal cancer markers also include truncated nucleic acids or nucleic acid fragments and variant forms of the nucleic acids that arise by alternative splicing of an mRNA corresponding to a DNA.

miRNA renal cancer markers also encompass precursors forms, including without limitation pri-miRNA and pre-miRNA, and polynucleotide encoding for such miRNA renal cancer marker.

The renal cancer polynucleotide markers are intended to include DNA and RNA, including without limitation mRNA and miRNA, and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method. In certain applications the term also includes antisense polynucleotides (e.g., RNA or DNA strand with complementary sequences in the reverse orientation to the protein renal cancer polynucleotide markers or miRNA renal cancer markers).

“Statistically different levels”, “significantly altered levels”, or “significant difference” in levels of markers in a patient sample compared to a control or standard (e.g., normal levels or levels in other samples from a patient) may represent levels that are higher or lower than the standard error of the detection assay. In particular embodiments, the levels may be 1.5, 2, 3, 4, 5, or 6 times higher or lower than the control or standard. In other embodiments of the invention, the levels may be significantly higher compared to the control or standard. In another embodiment, the levels may be significantly lower compared to the control or standard.

“Microarray” and “array” refer to nucleic acid or nucleotide arrays or protein or peptide arrays that can be used to detect biomolecules associated with renal cancer cell or tissue phase and renal disease, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Oligonucleotide probes that are 20- or 25-base long can be synthesized in silico on the array substrate. These arrays can achieve high densities (e.g., more than 40,000 genes per cm2). Generally spotted arrays have lower densities, but the probes, typically partial cDNA molecules, are much longer than 20- or 25-mers. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known (see for example, Zhu et al., Science 293:2101 (2001)). miRNA microarrays for the detection of miRNA are also known (see for example NCode™ Human miRNA Microarray Kit made by Invitrogen Inc.).

“Binding agent” refers to a substance such as a polypeptide or antibody that specifically binds to one or more renal cancer markers. A substance “specifically binds” to one or more protein renal cancer markers if it reacts at a detectable level with one or more renal cancer markers, and does not react detectably with peptides containing an unrelated or different sequence. Binding properties may be assessed using an ELISA, which may be readily performed by those skilled in the art (see, for example, Newton et al., Develop. Dynamics 197: 1-13, 1993).

A binding agent may be a ribosome, with or without a peptide component, an aptamer, an RNA molecule, or a polypeptide. A binding agent may be a polypeptide that includes one or more renal cancer marker sequence, a peptide variant thereof, or a non-peptide mimetic of such a sequence. By way of example, an YWHAH sequence may be a peptide portion of an YWHAH that is capable of modulating a function mediated by YWHAH.

An aptamer includes a DNA or RNA molecule that binds to nucleic acids and proteins. An aptamer that binds to a protein (or binding domain) of a renal cancer marker or a protein renal cancer polynucleotide marker can be produced using conventional techniques, without undue experimentation. For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996).

Antibodies for use in the present invention include but are not limited to monoclonal or polyclonal antibodies, immunologically active fragments (e.g., a Fab or (Fab)₂ fragments), antibody heavy chains, humanized antibodies, antibody light chains, genetically engineered single chain Fv molecules (Ladner et al., U.S. Pat. No. 4,946,778), chimeric antibodies, for example, antibodies which contain the binding specificity of murine antibodies, but in which the remaining portions are of human origin, or derivatives, such as enzyme conjugates or labelled derivatives.

Antibodies, including monoclonal and polyclonal antibodies, fragments and chimeras, may be prepared using methods known to those skilled in the art. Isolated native or recombinant renal cancer markers may be utilized to prepare antibodies. (See, for example, Kohler et al. (1975), Nature 256:495-497; Kozbor et al. (1985), J. Immunol Methods 81:31-42; Cote et al. (1983), Proc Natl Acad Sci 80:2026-2030; and Cole et al. (1984), Mol Cell Biol 62:109-120 for the preparation of monoclonal antibodies; Huse et al. (1989), Science 246:1275-1281 for the preparation of monoclonal Fab fragments; and Pound (1998), Immunochemical Protocols, Humana Press, Totowa, N.J. for the preparation of phagemid or B-lymphocyte immunoglobulin libraries to identify antibodies). Antibodies specific for a renal cancer marker may also be obtained from scientific or commercial sources.

In an embodiment of the invention, antibodies are reactive against a protein renal cancer marker if they bind with a Ka of greater than or equal to 10-7 M.

Markers. The invention provides a set of markers correlated with renal disease. In an aspect, the invention provides a set of markers identified as useful for detection, diagnosis, prevention and therapy of renal disease including or consisting of one or more of the markers listed in Table 8 and/or Table 12. In another aspect, the invention provides the renal cancer markers in Table 8 and/or Table 12 for detection, diagnosis and prognosis of a renal disease. The invention also provides a method of using renal cancer markers listed in Table 2 or Table 8 or Table 9 or Table 12 to distinguish renal disease.

In an embodiment, the markers include or consist of 1, 2, 3, 4 or more other markers listed in Table 2, Table 4, Table 5, Table 6, Table 7, or Table 9, and preferably Table 8 or Table 12.

In embodiments of the invention, a marker is provided which is selected from the group consisting of the polypeptides set forth in Table 8 which polypeptides are up-regulated biomarkers in renal. In embodiments of the invention, a marker is provided which is selected from the group consisting of at least one marker of Table 8.

In embodiments of the invention, a marker is provided which is selected from the group consisting of at least one marker of Table 8 and at least 2, 3, 4, 5, 6, 7, 8, 9, or 10 polypeptides set forth in Table 8.

In embodiments of the invention, a marker is provided which is selected from the group consisting of the renal cancer markers set forth in Table 12 which miRNA are up-regulated biomarkers in renal cancer cells. In embodiments of the invention, a marker is provided which is selected from the group consisting of at least one marker of Table 12 or a precursor thereof.

In embodiments of the invention, a marker is provided which is selected from the group consisting of at least one marker of Table 12 or a precursor thereof and at least 2, 3, 4, 5, 6, 7, 8, 9, or 10 miRNA set forth in Table 12 or a precursor thereof.

The invention provides marker sets that distinguish renal disease and uses therefor. In an aspect, the invention provides a method for classifying a renal disease including detecting a difference in the expression of a first plurality of renal cancer markers or polynucleotide encoding for the renal cancer markers relative to a control, the first plurality of renal cancer markers or polynucleotide encoding for the renal cancer including or consisting of at least 2, 3, 4, or 5 of the markers listed in Table 8 or miRNA renal cancer marker including or consisting of at least 2, 3, 4, or 5 of the markers listed in Table 12 or a precursor thereof. In specific aspects, a control includes markers derived from a sample from a patient with no renal disease.

Any of the markers provided herein may be used alone or with other markers of renal disease, or with markers for other phenotypes or conditions. Additionally, all of the sequences provided herein are representative only; there may be other sequences for particular protein or coding sequences or related sequences. The invention is not intended to be limited to the sequences herein provided.

Detection Methods. A variety of methods can be employed for the diagnostic and prognostic evaluation of renal cancer status involving one or more renal cancer markers and the polynucleotides encoding the markers, and the identification of subjects with a predisposition to renal diseases or that are receptive to in vitro fertilization and embryo transfer procedures. Such methods may, for example, involve renal cancer polynucleotide markers, and fragments thereof, and binding agents (e.g., antibodies) against one or more renal cancer markers, including peptide fragments. In particular, the polynucleotides and antibodies may be used, for example, for: (1) the detection of the presence of miRNA renal cancer markers or protein renal cancer polynucleotide marker mutations, or the detection of either over- or under-expression of protein renal cancer marker mRNA or miRNA renal cancer marker relative to a non-disorder state or different renal cancer cell or tissue phase, or the qualitative or quantitative detection of alternatively spliced forms of protein renal cancer polynucleotide marker transcripts which may correlate with certain conditions or susceptibility toward such conditions; and (2) the detection of either an over- or an under-abundance of one or more renal cancer markers relative to a non-disorder state or a different renal cell or tissue phase or the presence of a modified (e.g., less than full length) renal cancer which correlates with a disorder state or a progression toward a disorder state.

Recent advances in MS techniques enable proteins from different samples to be compared via labeled tags differing in isotopic composition. Samples are combined and processed in a single batch, allowing performance of comparative quantification (15). Effective labeling strategies include isotope-coded affinity tag (ICAT) (16), or the more recent variation that uses isobaric tagging reagent, iTRAQ (17), followed by multidimensional LC-MS/MS analysis. These approaches have been applied successfully to identify new tumor markers for endometrial cancer, pre-malignant lesions, and squamous cell carcinoma of head and neck (8, 9).

The invention contemplates a method for detecting the phase of a kidney tissue, in particular a secretory renal tissue, including producing a profile of levels of one or more renal cancer associated with a known renal cancer marker and/or polynucleotides encoding the markers, and optionally other markers associated with the phase in cells from a patient, and comparing the profile with a reference to identify a profile for the test cells indicative of the phase. In an aspect, renal cancer markers include those of Table 8, Table 12 or a precursor thereof, or fragments thereof.

The invention also contemplates a method for detecting a renal disease, in particular a renal cancer, including producing a profile of levels of one or more renal cancer marker associated with a renal disease and/or polynucleotides encoding the markers, and other markers associated with renal disease in cells from a patient, and comparing the profile with a reference to identify a profile for the test cells indicative of disease. In an aspect, the renal cancer markers are one or more of those listed in Table 8 or Table 12 or a precursor thereof.

The methods described herein may be used to evaluate the probability of the presence of malignant cells, for example, in a group of cells freshly removed from a host. Such methods can be used to detect tumors, quantify their growth, and help in the diagnosis and prognosis of renal disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring renal tissue status or a renal disease by detecting one or more renal cancer markers or polynucleotides encoding the markers in biological samples from a subject. These applications require that the amount of markers or polynucleotides quantified in a sample from a subject being tested be compared to a predetermined standard or cut-off value. The standard may correspond to levels quantified for another sample or an earlier sample from the subject, or levels quantified for a control sample. Levels for control samples from healthy subjects, different renal tissue phases, or subjects with a renal disease may be established by prospective and/or retrospective statistical studies. Healthy subjects who have no clinically evident disease or abnormalities may be selected for statistical studies. Diagnosis may be made by a finding of statistically different levels of detected renal cancer markers associated with disease or polynucleotides encoding same, compared to a control sample or previous levels quantified for the same subject.

The methods described herein may also use multiple markers for a renal disease, in particular renal cancer, RCC, or similar conditions. Therefore, the invention contemplates a method for analyzing a biological sample for the presence of one or more renal cancer markers and polynucleotides encoding the markers, and other markers that are specific indicators of a renal disease. The methods described herein may be modified by including reagents to detect the additional markers, or polynucleotides for the markers.

Nucleic Acid Methods/Assays. As noted herein a renal disease or phase may be detected based on the level of miRNA renal cancer markers or renal cancer polynucleotide markers in a sample. Techniques for detecting polynucleotides such as polymerase chain reaction (PCR) and hybridization assays are well known in the art.

Probes may be used in hybridization techniques to detect renal cancer polynucleotide markers. The technique generally involves contacting and incubating nucleic acids (e.g., recombinant DNA molecules, cloned genes) obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of a protein renal cancer polynucleotide marker, preferably they include 10-200, more particularly 10-30, 10-40, 20-50, 40-80, 50-150, 80-120 nucleotides in length.

The probes may include DNA or DNA mimics (e.g., derivatives and analogues) corresponding to a portion of an organism's genome, or complementary RNA or RNA mimics. Mimics are polymers including subunits capable of specific, Watson-Crick-like hybridization with DNA, or of specific hybridization with RNA. The nucleic acids can be modified at the base moiety, at the sugar moiety, or at the phosphate backbone.

DNA can be obtained using standard methods such as polymerase chain reaction (PCR) amplification of genomic DNA or cloned sequences. (See, for example, in Innis et al., eds., 1990, PCR Protocols: A Guide to Methods and Applications, Academic Press Inc., San Diego, Calif.). Computer programs known in the art can be used to design primers with the required specificity and optimal amplification properties, such as Oligo version 5.0 (National Biosciences). Controlled robotic systems may be useful for isolating and amplifying nucleic acids.

A nucleotide probe may be labelled with a detectable substance such as a radioactive label that provides for an adequate signal and has sufficient half-life such as 32P, 3H, 14C or the like. Other detectable substances that may be used include antigens that are recognized by a specific labelled antibody, fluorescent compounds, enzymes, antibodies specific for a labelled antigen, and luminescent compounds. An appropriate label may be selected having regard to the rate of hybridization and binding of the probe to the nucleotide to be detected and the amount of nucleotide available for hybridization. Labelled probes may be hybridized to nucleic acids on solid supports such as nitrocellulose filters or nylon membranes as generally described in Sambrook et al., 1989, Molecular Cloning, A Laboratory Manual (2nd ed.). The nucleic acid probes may be used to detect renal cancer polynucleotide markers, preferably in human cells. The nucleotide probes may also be useful in the diagnosis of a renal disease involving one or more renal cancer polynucleotide markers, in monitoring the progression of such disorder, or monitoring a therapeutic treatment.

The detection of miRNA renal cancer markers or polynucleotide encoding for the markers may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a polynucleotide encoding one or more renal disease marker derived from a sample, wherein at least one of the oligonucleotide primers is specific for (i.e., hybridizes to) a polynucleotide encoding the renal disease marker. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a polynucleotide encoding a renal disease marker; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of miRNA renal cancer markers and protein renal cancer polynucleotide marker expression. For example, RNA may be isolated from a cell type or tissue known to express a renal cancer polynucleotide marker and tested utilizing the hybridization (e.g., standard Northern analyses) or PCR techniques referred to herein.

The primers and probes may be used in the above-described methods in situ (i.e., directly on tissue sections (fixed and/or frozen) of patient tissue obtained from biopsies or resections).

In an aspect of the invention, a method is provided employing reverse transcriptase-polymerase chain reaction (RT-PCR), in which PCR is applied in combination with reverse transcription. Generally, RNA is extracted from a sample tissue using standard techniques (e.g., guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a renal disease marker sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis. Real time RT-PCR and qRT-PCR may also be used.

Amplification may be performed on samples obtained from a subject with a suspected renal disease and an individual who is not afflicted with a renal disease. The reaction may be performed on several dilutions of cDNA spanning at least two orders of magnitude. A statistically significant difference in expression in several dilutions of the subject sample as compared to the same dilutions of the non-disease sample may be considered positive for the presence of a renal disease.

In an embodiment, the invention provides methods for determining the presence or absence of a renal disease in a subject including (a) contacting a sample obtained from the subject with oligonucleotides that hybridize to miRNA renal cancer markers or polynucleotide encoding for the renal cancer markers; and (b) detecting in the sample a level of nucleic acids that hybridize to the polynucleotides relative to a predetermined cut-off value, and therefrom determining the presence or absence of a renal disease in the subject. In an aspect, the renal disease is cancer and renal cancer markers are one or more of those listed in Table 8 or Table 12 or a precursor thereof.

The invention provides a method wherein mRNA for a protein renal cancer marker or miRNA renal cancer marker is detected by: (a) isolating RNA from a sample and combining the RNA with reagents to convert it to cDNA; (b) treating the converted cDNA with amplification reaction reagents and nucleic acid primers that hybridize to one or more miRNA renal cancer markers or mRNA encoding protein renal disease markers, to produce amplification products; (d) analyzing the amplification products to detect amounts of miRNA renal cancer marker or mRNA encoding protein renal disease markers; and (e) comparing the amount of such RNA to an amount detected against a panel of expected values for normal and malignant tissue derived using similar nucleic acid primers.

Renal cancer marker-positive samples or alternatively higher levels in patients compared to a control (e.g., non-cancerous tissue) may be indicative of late stage disease, and/or that the patient is not responsive to chemotherapy. Alternatively, negative samples or lower levels compared to a control (e.g., non-cancerous tissue or negative samples) may be indicative of progressive disease and shorter overall survival.

In another embodiment, the invention provides methods for determining the presence or absence of renal cancer in a subject including: (a) contacting a sample obtained from the subject with oligonucleotides that hybridize to one or more miRNA renal cancer markers or protein renal cancer polynucleotide markers; and (b) detecting in the sample levels of nucleic acids that hybridize to the polynucleotides relative to a predetermined cut-off value, and therefrom determining the presence or absence of renal cancer in the subject. In an embodiment, the renal cancer polynucleotide markers encode one or more polypeptides listed in Table 8.

In particular, the invention provides a method wherein YWHAH, CAPNS1, KNG1, and/or SERPING1 mRNA is detected by: (a) isolating mRNA from a sample and combining the mRNA with reagents to convert it to cDNA; (b) treating the converted cDNA with amplification reaction reagents and nucleic acid primers that hybridize to a polynucleotide encoding YWHAH, CAPNS1, KNG1, and/or SERPING1, to produce amplification products; (d) analyzing the amplification products to detect an amount of mRNA encoding YWHAH, CAPNS1, KNG1, and/or SERPING1; and (e) comparing the amount of mRNA to an amount detected against a panel of expected values for normal and malignant tissue derived using similar nucleic acid primers.

Protein ren-positive samples or alternatively higher levels, in particular significantly higher levels of YWHAH, CAPNS1, KNG1, and/or SERPING1 polynucleotides in patients compared to a control (e.g., normal or benign) are indicative of renal cell carcinoma. Negative samples or lower levels (e.g., of F2, PTMA polynucleotides) compared to a control (e.g., normal or benign) may also be indicative of progressive disease and poor overall survival.

Oligonucleotides or longer fragments derived from a renal cancer polynucleotide marker may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan et al. (1995), U.S. Pat. No. 5,474,796; Schena et al. (1996), Proc. Natl. Acad. Sci. 93:10614-10619; Baldeschweiler et al. (1995), PCT Application WO 95/251116; Shalon et al. (1995), PCT application WO 95/35505; Heller et al. (1997), Proc. Natl. Acad. Sci., 94:2150-2155; and Heller et al. (1997), U.S. Pat. No. 5,605,662).

Thus, the invention also includes an array including one or more renal cancer polynucleotide markers (in particular the markers listed in Table 8) or miRNA renal cancer markers or a precursor thereof (in particular the markers listed in Table 12 and more specifically in Table 9), and/or markers listed in Table 4 or Table 5 or Table 6 or Table 9 or Table 10 or Table 11 other markers. The array can be used to assay expression of renal cancer polynucleotide markers in the array. The invention allows the quantification of expression of one or more renal cancer polynucleotide markers.

Microarrays typically contain at separate sites nanomolar quantities of individual genes, cDNAs, or ESTs on a substrate (e.g., nitrocellulose or silicon plate), or photolithographically prepared glass substrate. The arrays are hybridized to cDNA probes using conventional techniques with gene-specific primer mixes. The target polynucleotides to be analyzed are isolated, amplified and labelled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Microarrays are prepared by selecting polynucleotide probes and immobilizing them to a solid support or surface. The probes may include DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods of the invention can be immobilized to a solid support or surface which may be either porous or non-porous. For example, the probes can be attached to a nitrocellulose or nylon membrane or filter covalently at either the 3′ or the 5′ end of the polynucleotide probe. The solid support may be a glass or plastic surface. In an aspect of the invention, hybridization levels are measured to microarrays of probes consisting of a solid support on the surface of which are immobilized a population of polynucleotides, such as a population of DNA or DNA mimics, or, alternatively, a population of RNA or RNA mimics. A solid support may be a nonporous or, optionally, a porous material such as a gel.

In accordance with embodiments of the invention, a microarray is provided including a support or surface with an ordered array of hybridization sites or “probes” each representing one of the markers described herein. The microarrays can be addressable arrays, and in particular positionally addressable arrays. Each probe of the array is typically located at a known, predetermined position on the solid support such that the identity of each probe can be determined from its position in the array. In preferred embodiments, each probe is covalently attached to the solid support at a single site.

Microarrays used in the present invention are preferably (a) reproducible, allowing multiple copies of a given array to be produced and easily compared with each other; (b) made from materials that are stable under hybridization conditions; (c) small (e.g., between 1 cm2 and 25 cm2, between 12 cm2 and 13 cm2, or 3 cm2); and (d) include a unique set of binding sites that will specifically hybridize to the product of a single gene in a cell (e.g., to a specific mRNA, miRNA, or to a specific cDNA derived therefrom). However, it will be appreciated that larger arrays may be used particularly in screening arrays, and other related or similar sequences will cross hybridize to a given binding site.

In accordance with an aspect of the invention, the microarray is an array in which each position represents one of the markers described herein (e.g., the markers listed in Table 8 or Table 12 and, optionally, Table 2, Table 4, Table 5, Table 6, and Table 7 or Table 9, Table 10, and Table 11, respectively). Each position of the array can include a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the renal cancer markers and renal cancer polynucleotide markers are present on the array. In a preferred embodiment, the array includes at least 5 of the renal cancer polynucleotide markers.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization (see, e.g., Egholm et al., 1993, Nature 363:566-568; U.S. Pat. No. 5,539,083).

Probes can be selected using an algorithm that takes into account binding energies, base composition, sequence complexity, cross-hybridization binding energies, and secondary structure (see Friend et al., WO 01/05935).

Positive control probes (e.g., probes known to be complementary and hybridize to sequences in the target polynucleotides) and negative control probes (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA. (See, also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing thousands of oligonucleotides complementary to defined sequences, at defined locations on a surface can be produced using photolithographic techniques for synthesis in situ (see Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684). In an embodiment, microarrays of the present invention are produced by synthesizing polynucleotide probes on a support wherein the nucleotide probes are attached to the support covalently at either the 3′ or the 5′ end of the polynucleotide.

The invention provides microarrays including a disclosed marker set. In one embodiment, the invention provides a microarray for distinguishing renal disease samples including a positionally-addressable array of polynucleotide probes bound to a support, the polynucleotide probes including a plurality of polynucleotide probes of different nucleotide sequences, each of the different nucleotide sequences including a sequence complementary and hybridizable to a plurality of genes, the plurality consisting of at least 2, 3, 4, 5, or 6 of the genes corresponding to the markers listed in Table 8 and optionally at least 2 to 18, 5 to 16, 10 to 15, 13-21, 2-21, 2-32, 22-32 or 13-32 of the genes corresponding to the markers listed in Table 8. An aspect of the invention provides microarrays including at least 4, 5, or 6 of the polynucleotides encoding the markers listed in Table 8 or the miRNA renal cancer markers listed in Table 12 or a precursor thereof.

The invention provides gene marker sets that distinguish renal disease and uses therefor. In an aspect, the invention provides a method for classifying a renal disease including detecting a difference in the expression of a first plurality of genes relative to a control, the first plurality of genes consisting of at least 3, 4, 5, or 6 of the genes encoding the markers listed in Table 8 or Table 12. In specific aspects, the plurality of genes consists of at least 4 or 5 of the genes encoding the markers listed in Table 8 and optionally at least 2 to 18, 5 to 16, 10 to 15-21, 2-21, 2-32, 22-32, or 13-32 of the genes corresponding to the markers listed in Table 2. In another specific aspect, the control includes nucleic acids derived from a pool of samples from individual control patients. An aspect of the invention provides microarrays including at least 4, 5, or 6 of the miRNA renal cancer markers listed in Table 12 or a precursor thereoft or the polynucleotides encoding the markers listed in Table 8.

The invention provides a method for classifying a renal disease by calculating the similarity between the expression of at least 3, 4, 5, or 6 miRNA renal cancer markers or a precursor thereof or Table 8, respectively, in a sample to the expression of the same markers in a control pool, by: (a) labelling nucleic acids derived from a sample, with a first fluorophore to obtain a first pool of fluorophore-labelled nucleic acids; (b) labelling with a second fluorophore a first pool of nucleic acids derived from two or more renal disease samples, and a second pool of nucleic acids derived from two or more control samples; (c) contacting the first fluorophore-labelled nucleic acid and the first pool of second fluorophore-labelled nucleic acid with a first microarray under conditions such that hybridization can occur, and contacting the first fluorophore-labelled nucleic acid and the second pool of second fluorophore-labelled nucleic acid with a second microarray under conditions such that hybridization can occur, detecting at each of a plurality of discrete loci on the first microarray a first fluorescent emission signal from the first fluorophore-labelled nucleic acid and a second fluorescent emission signal from the first pool of second fluorophore-labelled genetic matter that is bound to the first microarray and detecting at each of the marker loci on the second microarray the first fluorescent emission signal from the first fluorophore-labelled nucleic acid and a third fluorescent emission signal from the second pool of second fluorophore-labelled nucleic acid; (d) determining the similarity of the sample to patient and control pools by comparing the first fluorescence emission signals and the second fluorescence emission signals, and the first emission signals and the third fluorescence emission signals; and (e) classifying the sample as renal disease where the first fluorescence emission signals are more similar to the second fluorescence emission signals than to the third fluorescent emission signals, and classifying the sample as non-renal disease where the first fluorescence emission signals are more similar to the third fluorescence emission signals than to the second fluorescent emission signals, wherein the first microarray and the second microarray are similar to each other, exact replicas of each other, or are identical, and wherein the similarity is defined by a statistical method such that the cell sample and control are similar where the p value of the similarity is less than 0.01.

In aspects of the invention, the array can be used to monitor the time course of expression of one or more renal cancer polynucleotide markers in the array. This can occur in various biological contexts such as tumor progression.

The array is also useful for ascertaining differential expression patterns of renal cancer polynucleotide markers, and optionally other markers, in normal and abnormal cells. This may provide a battery of nucleic acids that could serve as molecular targets for diagnosis or therapeutic intervention.

Protein Methods. Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of a renal disease (e.g., cancer) in a subject may be determined by: (a) contacting a sample from the subject with a binding agent; (b) detecting in the sample a level of polypeptide that binds to the binding agent; and (c) comparing the level of polypeptide with a predetermined standard or cut-off value.

In particular embodiments of the invention, the binding agent is an antibody. Antibodies specifically reactive with one or more renal disease marker, or derivatives, such as enzyme conjugates or labelled derivatives, may be used to detect one or more renal disease marker in various samples (e.g., biological materials). They may be used as diagnostic or prognostic reagents and they may be used to detect abnormalities in the level of expression of one or more renal disease marker, or abnormalities in the structure, and/or temporal, tissue, cellular, or subcellular location of one or more renal disease marker. Antibodies may also be used to screen potentially therapeutic compounds in vitro to determine their effects on disorders (e.g., cancer) involving one or more renal cancer markers, and other conditions. In vitro immunoassays may also be used to assess or monitor the efficacy of particular therapies.

In an aspect, the invention provides a method for monitoring or diagnosing a renal disease (e.g., cancer) in a subject by quantifying one or more renal cancer markers in a biological sample from the subject including reacting the sample with antibodies specific for one or more renal cancer markers, which are directly or indirectly labelled with detectable substances and detecting the detectable substances. In a particular embodiment of the invention, renal cancer markers are quantified or measured.

In an aspect of the invention, a method for detecting or screening for a renal disease (e.g., cancer) is provided by: (a) obtaining a sample suspected of containing one or more protein renal cancer markers associated with a renal disease; (b) contacting the sample with antibodies that specifically bind to the protein renal cancer markers under conditions effective to bind the antibodies and form complexes; (c) measuring the amount of protein renal cancer markers present in the sample by quantifying the amount of the complexes; and (d) comparing the amount of protein renal cancer markers present in the samples with the amount of protein renal cancer markers in a control, wherein a change or significant difference in the amount of protein renal cancer markers in the sample compared with the amount in the control is indicative of a renal disease.

In an embodiment, the invention contemplates a method for monitoring the progression of a renal disease (e.g., cancer) in an individual, by: (a) contacting antibodies which bind to one or more protein renal cancer markers with a sample from the individual so as to form complexes including the antibodies and one or more renal cancer markers in the sample; (b) determining or detecting the presence or amount of complex formation in the sample; (c) repeating steps (a) and (b) at a point later in time; and (d) comparing the result of step (b) with the result of step (c), wherein a difference in the amount of complex formation is indicative of disease, disease stage, and/or progression of the disease in the individual.

The amount of complexes may also be compared to a value representative of the amount of the complexes from an individual not at risk of, or afflicted with, a renal disease at different stages. A significant difference in complex formation may be indicative of advanced disease (e.g., advanced renal cancer, or an unfavourable prognosis).

In aspects of the invention for diagnosis and monitoring of renal cancer, the renal cancer markers are one or more of those upregulated in cancer samples as compared to normal samples in Table 8, and/or YWHAH, CAPNS1, KNG1 and/or SERPING1.

In embodiments of the methods of the invention, YWHAH, CAPNS1, KNG1, and/or SERPING1 is detected in samples and higher levels, in particular significantly higher levels compared to a control (normal or benign) is indicative of the prognosis of renal cancer patient outcome.

In aspects of the invention for characterizing renal disease, the renal cancer markers include YWHAH, CAPNS1, KNG1, and/or SERPING1 and fragments thereof.

Antibodies may be used in any known immunoassays that rely on the binding interaction between antigenic determinants of one or more renal disease marker and the antibodies. Immunoassay procedures for in vitro detection of antigens in fluid samples are also well known in the art. (See, for example, Paterson et al., Int. J. Can. 37:659 (1986) and Burchell et al., Int. J. Can. 34:763 (1984) for a general description of immunoassay procedures.) Qualitative and/or quantitative determinations of one or more renal disease marker in a sample may be accomplished by competitive or non-competitive immunoassay procedures in either a direct or indirect format. Detection of one or more renal disease marker using antibodies can be done utilizing immunoassays which are run in either the forward, reverse or simultaneous modes. Examples of immunoassays are radioimmunoassays (RIA), enzyme immunoassays (e.g., ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

According to an embodiment of the invention, an immunoassay for detecting one or more renal cancer markers in a biological sample includes contacting binding agents that specifically bind to renal cancer markers in the sample under conditions that allow the formation of first complexes including a binding agent and renal cancer markers and determining the presence or amount of the complexes as a measure of the amount of renal cancer markers contained in the sample. In a particular embodiment, the binding agents are labelled differently or are capable of binding to different labels.

Antibodies may be used to detect and quantify one or more renal cancer markers in a sample in order to diagnose and treat pathological states. In particular, the antibodies may be used in immunohistochemical analyses, for example, at the cellular and sub-subcellular level, to detect one or more renal cancer markers, to localize them to particular renal cells and tissues (e.g., tumor cells and tissues), and to specific subcellular locations, and to quantify the level of expression.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having a renal-related problem is contacted with antibodies, preferably monoclonal antibodies recognizing one or more renal cancer markers. The site at which the antibodies are bound is determined by selective staining of the sample by standard immunohistochemical procedures. The same procedure may be repeated on the same sample using other antibodies that recognize one or more renal cancer markers. Alternatively, a sample may be contacted with antibodies against one or more renal cancer markers simultaneously, provided that the antibodies are labelled differently or are able to bind to a different label. The tissue sample may be normal renal tissue, or a cancer tissue or a benign tissue.

An antibody microarray in which binding sites include immobilized, preferably monoclonal, antibodies specific to a substantial fraction of marker-derived renal cancer markers of interest can be utilized in the present invention. Antibody arrays can be prepared using methods known in the art (see, for example, Zhu et al., Science 293:2101 (2001) and reference 20).

Antibodies specific for one or more renal marker may be labelled with a detectable substance and localised in biological samples based upon the presence of the detectable substance. Examples of detectable substances include, but are not limited to, the following: radioisotopes (e.g., 3H, 14C, 35S, 125I, 131I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin (e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labelled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against one or more renal cancer markers. By way of example, if the antibody having specificity against one or more renal disease marker is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See, for example, Inman, Methods In Enzymology, vol. 34, “Affinity Techniques, Enzyme Purification: Part B”, Jakoby and Wichek (eds.), Academic Press, New York, p. 30, 1974; and Wilchek and Bayer, “The Avidin-Biotin Complex in Bioanalytical Applications”, Anal. Biochem. 171:1-32, 1988 re methods for conjugating or labelling the antibodies with enzyme or ligand binding partner.)

Cytochemical techniques known in the art for localizing antigens using light and electron microscopy may be used to detect one or more renal cancer markers. Generally, antibodies may be labelled with detectable substances and one or more renal cancer markers may be localised in tissues and cells based upon the presence of the detectable substances.

In the context of the methods of the invention, the sample, binding agents (e.g., antibodies specific for one or more renal cancer markers), or one or more renal cancer markers may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g., bead), cylindrical (e.g., inside surface of a test tube or well, or the external surface of a rod), or flat (e.g., sheet, test strip). Thus, the carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling. An antibody may be indirectly immobilized using a second antibody specific for the antibody. For example, mouse antibody specific for a renal disease marker may be immobilized using sheep anti-mouse IgG Fc fragment specific antibody coated on the carrier or support.

Where a radioactive label is used as a detectable substance, one or more renal disease marker may be localized by radioautography. The results of radioautography may be quantified by determining the density of particles in the radioautographs by various optical methods, or by counting the grains.

Time-resolved fluorometry may be used to detect a signal. For example, the method described by Christopoulos and Diamandis, in Anal. Chem. 1992:64:342-346 may be used with a conventional time-resolved fluorometer.

In accordance with an embodiment of the invention, a method is provided wherein one or more renal disease marker antibodies are directly or indirectly labelled with enzymes, substrates for the enzymes are added wherein the substrates are selected so that the substrates, or a reaction product of an enzyme and substrate, form fluorescent complexes with a lanthanide metal (e.g., europium, terbium, samarium, and dysprosium, preferably europium and terbium). A lanthanide metal is added and one or more renal cancer markers are quantified in the sample by measuring fluorescence of the fluorescent complexes. Enzymes are selected based on the ability of a substrate of the enzyme, or a reaction product of the enzyme and substrate, to complex with lanthanide metals such as europium and terbium. Suitable enzymes and substrates that provide fluorescent complexes are described in U.S. Pat. No. 5,3112,922 to Diamandis. Examples of suitable enzymes include alkaline phosphatase and β-galactosidase. Preferably, the enzyme is alkaline phosphatase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer (e.g., a CyberFluor 615 Immunoanalyzer (Nordion International, Kanata, Ontario)).

One or more renal disease marker antibodies may also be indirectly labelled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for renal disease marker antibody is labelled with an enzyme.

In accordance with an embodiment, the present invention provides means for determining one or more renal cancer markers in a sample by measuring one or more renal cancer markers by immunoassay. It will be evident to a skilled artisan that a variety of immunoassay methods can be used to measure one or more renal cancer markers. In general, an immunoassay method may be competitive or non-competitive. Competitive methods typically employ an immobilized or immobilizable antibody to one or more renal disease marker and a labelled form of one or more renal disease marker. Sample renal disease cancer markers and labelled renal cancer markers compete for binding to antibodies to renal cancer markers. After separation of the resulting labelled renal cancer markers that have become bound to antibodies (bound fraction) from that which has remained unbound (unbound fraction), the amount of the label in either bound or unbound fraction is measured and may be correlated with the amount of renal cancer markers in the test sample in any conventional manner (e.g., by comparison to a standard curve).

In an aspect, a non-competitive method is used for the determination of one or more renal cancer markers, with the most common method being the “sandwich” method. In this assay, two antibodies to renal cancer markers are employed. One of the antibodies to renal cancer markers is directly or indirectly labelled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally, it is measured in the capture antibody phase since it includes renal disease markers bound by (“sandwiched” between) the capture and detection antibodies. In an embodiment, the label may be measured without separating the capture antibodies and liquid test mixture.

In a typical two-site immunometric assay for renal cancer markers, one or both of the capture and detection antibodies are polyclonal antibodies or one or both of the capture and detection antibodies are monoclonal antibodies (i.e., polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labelled binding partner for the ligand), and the like. In a particular aspect, the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may include an antibody covalently or non-covalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

The above-described immunoassay methods and formats are intended to be exemplary and are not limiting.

Computer Systems. Analytic methods contemplated herein can be implemented by use of computer systems and methods described below and known in the art. Thus, the invention provides computer readable media including one or more renal cancer markers, and/or polynucleotides encoding one or more renal cancer markers, and optionally other markers (e.g., markers of renal cancer). “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures including information on one or more renal cancer markers, and optionally other markers.

A variety of data processor programs and formats can be used to store information on one or more renal cancer markers, and/or polynucleotides encoding one or more renal cancer markers, and other markers on computer readable medium. For example, the information can be represented in a word processing text file, formatted in commercially-available software such as WordPerfect and Microsoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining or whether a patient has a renal disease (e.g., renal cancer) or a pre-disposition to a renal disease (e.g., cancer), including determining the presence or absence of one or more renal cancer markers, and/or polynucleotides encoding one or more renal cancer markers, and optionally other markers, and based on the presence or absence of the one or more renal cancer markers, and/or polynucleotides encoding one or more renal cancer markers, and optionally other markers, determining uterine renal receptivity, renal disease (e.g., cancer) or a pre-disposition to a renal disease (e.g., cancer), and optionally recommending a procedure or treatment.

The invention also provides in an electronic system and/or in a network, a method for determining whether a subject has a renal disease (e.g., cancer) or a pre-disposition to a renal disease (e.g., cancer), including determining the presence or absence of one or more renal cancer markers and/or polynucleotides encoding one or more renal cancer markers, and optionally other markers (e.g., cancer markers), and based on the presence or absence of the one or more renal cancer markers and/or polynucleotides encoding one or more renal cancer markers, and optionally other markers, determining whether the subject has a renal disease (e.g., cancer) or a pre-disposition to a renal disease (e.g., cancer), and optionally recommending a procedure or treatment.

The invention further provides in a network, a method for determining whether a subject is receptive to in vitro fertilization, has a renal disease (e.g., cancer) or a pre-disposition to a renal disease (e.g., cancer) including: (a) receiving phenotypic information on the subject and information on one or more renal cancer markers, and/or polynucleotides encoding one or more renal cancer markers, and optionally other markers associated with samples from the subject; (b) acquiring information from the network corresponding to the one or more renal cancer markers, and/or polynucleotides encoding one or more renal cancer markers, and optionally other markers; and (c) based on the phenotypic information and information on the one or more renal cancer markers, and/or polynucleotides encoding one or more renal cancer markers, and optionally other markers, determining whether the subject is receptive to in vitro fertilization, has a renal disease (e.g., cancer) or a pre-disposition to a renal disease (e.g., cancer); and (d) optionally recommending a procedure or treatment.

The invention still further provides a system for identifying selected records that identify a diseased renal cell or tissue (e.g., cancer cell or tissue) or a renal cell or tissue phase. A system of the invention generally includes a digital computer; a database server coupled to the computer; a database coupled to the database server having data stored therein, the data including records of data including one or more renal cancer markers, and/or polynucleotides encoding one or more renal cancer markers, and optionally other renal cancer markers, and a code mechanism for applying queries based upon a desired selection criteria to the data file in the database to produce reports of records which match the desired selection criteria.

In an aspect of the invention a method is provided for detecting renal cancer tissue or cells using a computer having a processor, memory, display, and input/output devices, the method by: (a) creating records of one or more renal cancer markers, and/or polynucleotides encoding one or more renal cancer markers, and optionally other markers of cancer identified in a sample suspected of containing renal cancer cells or tissue; (b) providing a database including records of data including one or more renal cancer markers, and/or polynucleotides encoding one or more renal cancer markers, and optionally other markers of cancer; and (c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are renal cancer cells or renal cancer tissue.

The invention contemplates a business method for determining whether a subject is receptive to in vitro fertilization, has a renal disease (e.g., cancer) or a pre-disposition to renal cancer including: (a) receiving phenotypic information on the subject and information on one or more renal cancer markers, and/or polynucleotides encoding the markers, and optionally other markers, associated with samples from the subject; (b) acquiring information from a network corresponding to one or more renal cancer markers, and/or polynucleotides encoding the markers, and optionally other markers; and (c) based on the phenotypic information, information on one or more renal cancer markers, and/or polynucleotides encoding the markers, and optionally other markers, and acquired information, determining whether the subject is receptive to in vitro fertilization, has a renal disease (e.g., cancer) or a pre-disposition to a renal disease (e.g., cancer); and (d) optionally recommending a procedure or treatment. In one embodiment, the renal cancer markers are protein renal cancer markers. In another embodiment, the renal cancer markers are miRNA renal cancer markers.

In an aspect of the invention, the computer systems, components, and methods described herein are used to monitor disease or determine the stage of disease.

Imaging Methods. Binding agents, particularly antibodies, specific for one or more renal disease markers may also be used in imaging methodologies in the management of a renal disease.

In an aspect, the invention provides a method for imaging tumors associated with one or more renal cancer markers.

The invention also contemplates imaging methods described herein using multiple markers for a renal disease. Preferably, each agent is labelled so that it can be distinguished during the imaging.

In an embodiment the method is an in vivo method and a subject or patient is administered one or more agents that carry an imaging label and that are capable of targeting or binding to one or more renal cancer markers. The agent is allowed to incubate in vivo and bind to the renal cancer markers associated with renal cells or tissues of a particular phase or associated with diseased cells or tissues, (e.g., renal tumor). The presence of the label is localized to the renal cells or tissues, and the localized label is detected using imaging devices known to those skilled in the art.

The agent may be an antibody or chemical entity that recognizes the renal cancer markers. In an aspect of the invention the agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for the renal cancer markers used in the methods of the invention may be obtained from scientific or commercial sources, or isolated native renal cancer markers or recombinant renal cancer markers may be utilized to prepare antibodies, etc. as described herein.

An agent may be a peptide that mimics the epitope for an antibody specific for a renal disease marker and binds to the marker. The peptide may be produced on a commercial synthesizer using conventional solid phase chemistry. By way of example, a peptide may be prepared that includes tyrosine, lysine, or phenylalanine to which N2S2 chelate is complexed (see U.S. Pat. No. 4,897,255). An anti-endocrine marker peptide conjugate is then combined with a radiolabel (e.g., sodium 99 mTc pertechnetate or sodium 188Re perrhenate) and it may be used to locate a renal disease marker producing cell or tissue (e.g., tumor).

The agent carries a label to image the renal cancer markers. The agent may be labelled for use in radionuclide imaging. In particular, the agent may be directly or indirectly labelled with a radioisotope. Examples of radioisotopes that may be used in the present invention are the following: 277Ac, 211At, 128Ba, 131Ba, 7Be, 204Bi, 205Bi, 206Bi, 76Br, 77Br, 82Br, 109Cd, 47Ca, 11C, 14C, 36Cl, 48Cr, 51Cr, 62Cu, 64Cu, 67Cu, 165Dy, 155Eu, 18F, 153Gd, 66Ga, 67Ga, 68Ga, 72Ga, 198Au, 3H, 166Ho, 111In, 113mln, 115mln, 123I, 125I, 131I, 189lr, 191mlr, 192lr, 194lr, 52Fe, 55Fe, 59Fe, 177Lu, 15O, 191m-191Os, 109Pd, 32P, 33P, 42K, 226Ra, 186Re, 188Re, 82mRb, 153Sm, 46Sc, 47Sc, 72Se, 75Se, 105Ag, 22Na, 24Na, 89Sr, 35S, 38s, 177Ta, 96Tc, 99mTc, 201Tl, 202Tl, 113Sn, 117mSn, 121Sn, 166Yb, 169Yb, 175Yb, 88Y, 90Y, 62Zn and 65Zn. Preferably the radioisotope is 131I, 125I, 123I, 111I, 99mTc, 90Y, 186Re, 188Re, 32P, 153Sm, 67Ga, 201Tl 77Br, or 18F, and is imaged with a photoscanning device.

Procedures for biological agents with the radioactive isotopes are generally known in the art. U.S. Pat. No. 4,302,438 describes tritium procedures. Procedures for iodinating, tritium labelling, and 35S especially adapted for murine monoclonal antibodies are described by Goding, J. W. (supra, pp. 124-126) and the references cited therein. Other procedures for iodinating biological agents, such as antibodies, binding portions thereof, probes, or ligands, are described in the scientific literature (see Hunter and Greenwood, Nature 144:945 (1962); David et al., Biochemistry 13:1014-1021 (1974); and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). 99m Tc-labeling procedures are described by Rhodes, B. et al. in Burchiel, S. et al. (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. Nos. 5,317,091; 4,478,815; 4,478,818; 4,472,371; Re 32,417; and 4,311,688. Procedures suitable for 111 In-biological agents are described by Hnatowich et al., J. Immun. Methods, 65:147-157 (1983), Hnatowich et al., J. Applied Radiation, 35:554-557 (1984), and Buckley et al., F.E.B.S. 166:202-204 (1984).

An agent may also be labelled with a paramagnetic isotope for purposes of an in vivo method of the invention. Examples of elements that are useful in magnetic resonance imaging include gadolinium, terbium, tin, iron, or isotopes thereof. (See, for example, Schaefer et al., (1989) JACC 14, 472-480; Shreve et al., (1986) Magn. Reson. Med. 3, 336-340; Wolf, G. L., (1984) Physiol. Chem. Phys. Med. NMR 16, 93-95; Wesbey et al. (1984), Physiol. Chem. Phys. Med. NMR 16, 145-155; Runge et al. (1984), Invest. Radiol. 19, 408-415 for discussions on in vivo nuclear magnetic resonance imaging.)

In the case of a radiolabeled agent, the agent may be administered to the patient, it is localized to the cell or tissue (e.g., tumor) having a renal disease marker with which the agent binds, and is detected or “imaged” in vivo using known techniques such as radionuclear scanning using e.g., a gamma camera or emission tomography. (See, for example, “Developments in Antibody Imaging”, Monoclonal Antibodies for Cancer Detection and Therapy, Baldwin et al. (eds.), pp. 65-85 (Academic Press 1985).) A positron emission transaxial tomography scanner, such as designated Pet VI located at Brookhaven National Laboratory, can also be used where the radiolabel emits positrons (e.g., 11C, 18F, 15O, and 13N).

Whole body imaging techniques using radioisotope labelled agents can be used for locating diseased cells and tissues (e.g., primary tumors and tumors which have metastasized). Antibodies specific for renal cancer markers, or fragments thereof having the same epitope specificity, are bound to a suitable radioisotope, or a combination thereof, and administered parenterally. For renal cancer, administration preferably is intravenous. The bio-distribution of the label can be monitored by scintigraphy, and accumulations of the label are related to the presence of renal cancer cells. Whole body imaging techniques are described in U.S. Pat. Nos. 4,036,945 and 4,311,688. Other examples of agents useful for diagnosis and therapeutic use that can be coupled to antibodies and antibody fragments include metallothionein and fragments (see U.S. Pat. No. 4,732,864). These agents are useful in diagnosis staging and visualization of cancer, in particular renal cancer, so that surgical and/or radiation treatment protocols can be used more efficiently.

An imaging agent may carry a bioluminescent or chemiluminescent label. Such labels include polypeptides known to be fluorescent, bioluminescent, or chemiluminescent, or, that act as enzymes on a specific substrate (reagent), or can generate a fluorescent, bioluminescent or chemiluminescent molecule. Examples of bioluminescent or chemiluminescent labels include luciferases, aequorin, obelin, mnemiopsin, berovin, a phenanthridinium ester, and variations thereof and combinations thereof. A substrate for the bioluminescent or chemiluminescent polypeptide may also be utilized in a method of the invention. For example, the chemiluminescent polypeptide can be luciferase and the reagent luciferin. A substrate for a bioluminescent or chemiluminescent label can be administered before, at the same time (e.g., in the same formulation), or after administration of the agent.

An imaging agent may include a paramagnetic compound, such as a polypeptide chelated to a metal (e.g., a metalloporphyrin). The paramagnetic compound may also include a monocrystalline nanoparticle, e.g., a nanoparticle including a lanthanide (e.g., Gd) or iron oxide; or, a metal ion such as a lanthanide. As used herein, “lanthanide” refers to elements of atomic numbers 58 to 70, a transition metal of atomic numbers 21 to 29, 42 or 44, a Gd(III), a Mn(II), or an element including a Fe element. Paramagnetic compounds can also include a neodymium iron oxide (NdFeO3) or a dysprosium iron oxide (DyFeO3). Examples of elements that are useful in magnetic resonance imaging include gadolinium, terbium, tin, iron, or isotopes thereof. (See, for example, Schaefer et al., (1989) JACC 14, 472-480; Shreve et al. (1986), Magn. Reson. Med. 3, 336-340; Wolf, G. L. (1984), Physiol. Chem. Phys. Med. NMR 16, 93-95; Wesbey et al. (1984), Physiol. Chem. Phys. Med. NMR 16, 145-155; Runge et al. (1984), Invest. Radiol. 19, 408-415 for discussions on in vivo nuclear magnetic resonance imaging.)

An image can be generated in a method of the invention by computer assisted tomography (CAT), magnetic resonance spectroscopy (MRS) image, magnetic resonance imaging (MRI), positron emission tomography (PET), single-photon emission computed tomography (SPECT), or bioluminescence imaging (BLI) or equivalent.

Computer assisted tomography (CAT) and computerized axial tomography (CAT) systems and devices well known in the art can be utilized in the practice of the present invention. (See, for example, U.S. Pat. Nos. 6,151,377; 5,946,371; 5,446,799; 5,406,479; 5,208,581; and 5,109, 97.) The invention may also utilize animal imaging modalities, such as MicroCAT™ (ImTek, Inc.).

Magnetic resonance imaging (MRI) systems and devices well known in the art can be utilized in the practice of the present invention. For a description of MRI methods and devices, see, for example, U.S. Pat. Nos. 6,151,377; 6,144,202; 6,128,522; 6,127,825; 6,121,775; 6,119,032; 6,115,446; 6,111,410; ,602,891; 5,555,251; 5,455,512; 5,450,010; 5,378,987; 5,214,382; 5,031,624; 5,207,222; 4,985,678; 4,906,931; and 4,558,279. MRI and supporting devices are commercially available, for example, from Bruker Medical GMBH; Caprius; Esaote Biomedica; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems by, e.g., Silicon Graphics. The invention may also utilize animal imaging modalities such as micro-MRIs.

Positron emission tomography imaging (PET) systems and devices well known in the art can be utilized in the practice of the present invention. For example, a method of the invention may use the system designated Pet VI located at Brookhaven National Laboratory. For descriptions of PET systems and devices, see, for example, U.S. Pat. Nos. 6,151,377; 6,072,177; 5,900,636; 5,608,221; 5,532,489; 5,272,343; and 5,103,098. Animal imaging modalities such as micro-PETs (Concorde Microsystems, Inc.) can also be used in the invention.

Single-photon emission computed tomography (SPECT) systems and devices well known in the art can be utilized in the practice of the present invention. (See, for example, U.S. Pat. Nos. 6,115,446; 6,072,177; 5,608,221; 5,600,145; 5,210,421; 5,103,098.) The methods of the invention may also utilize animal imaging modalities, such as micro-SPECTs.

Bioluminescence imaging includes bioluminescence, fluorescence, and chemiluminescence and other photon detection systems and devices that are capable of detecting bioluminescence, fluorescence, or chemiluminescence. Sensitive photon detection systems can be used to detect bioluminescent and fluorescent proteins externally; see, for example, Contag (2000), Neoplasia 2:41-52; and Zhang (1994), Clin. Exp. Metastasis, 12:87-92. The methods of the invention can be practiced using any such photon detection device, or variation or equivalent thereof, or in conjunction with any known photon detection methodology, including visual imaging. By way of example, an intensified charge-coupled device (ICCD) camera coupled to an image processor may be used in the present invention. (See, e.g., U.S. Pat. No. 5,650,135.) Photon detection devices are also commercially available from Xenogen, Hamamatsue.

Screening Methods. The invention also contemplates methods for evaluating test agents or compounds for their ability to inhibit a renal disease (e.g., cancer), potentially contribute to a renal disease (e.g., cancer). Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies (e.g., polyclonal, monoclonal, humanized, anti-idiotypic, chimeric, single chain antibodies, fragments (e.g., Fab, F(ab)₂, and Fab expression library fragments, and epitope-binding fragments thereof), and small organic or inorganic molecules. The agents or compounds may be endogenous physiological compounds or natural or synthetic compounds.

The invention provides a method for assessing the potential efficacy of a test agent or therapy for inhibiting a renal disease (e.g., cancer) in a patient, by comparing: (a) levels of one or more protein renal cancer markers, miRNA renal cancer markers, and/or polynucleotides encoding renal cancer markers, and optionally other markers in a first sample obtained from a patient and exposed to the test agent or therapy; and (b) levels of one or more protein renal cancer markers, miRNA renal cancer markers, and/or polynucleotides encoding renal cancer markers, and optionally other markers, in a second sample obtained from the patient, wherein the sample is not exposed to the test agent or therapy, wherein a significant difference in the levels of expression of one or more protein renal cancer markers, miRNA renal cancer markers and/or polynucleotides encoding one or more renal cancer markers, and optionally the other markers, in the first sample, relative to the second sample, is an indication that the test agent or therapy is potentially efficacious for inhibiting a renal disease (e.g., cancer) in the patient.

The first and second samples may be portions of a single sample obtained from a patient or portions of pooled samples obtained from a patient.

In an aspect, the invention provides a method of selecting an agent for inhibiting a renal disease (e.g., cancer) in a patient by: (a) obtaining a sample from the patient; (b) separately maintaining aliquots of the sample in the presence of a plurality of test agents; (c) comparing one or more renal cancer markers, and/or polynucleotides encoding renal cancer markers, and optionally other markers, in each of the aliquots; and (d) selecting one of the test agents which alters the levels of one or more renal cancer markers, and/or polynucleotides encoding renal cancer markers, and optionally other markers in the aliquot containing that test agent, relative to other test agents.

In a further aspect, the invention provides a method of selecting an agent for inhibiting or enhancing renal cell or tissue phase in a patient by: (a) obtaining a sample of renal cell or tissue in a selected phase; (b) separately maintaining aliquots of the sample in the presence of a plurality of test agents; (c) comparing one or more renal cancer markers, and/or polynucleotides encoding renal cancer markers, and optionally other markers, in each of the aliquots; and (d) selecting one of the test agents which alters the levels of one or more renal cancer markers, and/or polynucleotides encoding renal cancer markers, and optionally other markers in the aliquot containing that test agent, relative to other test agents.

Still another aspect of the present invention provides a method of conducting a drug discovery business by: (a) providing one or more methods or assay systems for identifying agents that inhibit a renal disease (e.g., cancer) or affect a renal cell or tissues phase in a patient; (b) conducting therapeutic profiling of agents identified in step (a), or further analogs thereof, for efficacy and toxicity in animals; and (c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to a renal disease (e.g., cancer) by: (a) maintaining separate aliquots of cells or tissues from a patient with a renal disease (e.g., cancer) in the presence and absence of the test compound; and (b) comparing one or more renal cancer markers and/or polynucleotides encoding the renal cancer markers, and optionally other markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to a renal disease (e.g., cancer). In one embodiment, the renal cancer markers are protein renal cancer markers. In another embodiment, the renal cancer markers are miRNA renal cancer markers.

Kits. The invention also contemplates kits for carrying out the methods of the invention. Kits may typically include two or more components required for performing a diagnostic assay. Components include but are not limited to compounds, reagents, containers, and/or equipment.

The methods described herein may be performed by utilizing pre-packaged diagnostic kits including one or more specific renal disease marker polynucleotide, miRNA renal cancer markers, or antibody described herein, which may be conveniently used, e.g., in clinical settings to screen and diagnose patients and to screen and identify those individuals exhibiting a predisposition to developing a renal disease.

In an embodiment, a container with a kit includes a binding agent as described herein. By way of example, the kit may contain antibodies or antibody fragments which bind specifically to epitopes of one or more renal cancer markers and optionally other markers, antibodies against the antibodies labelled with an enzyme; and a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more polypeptide listed in Table 8 that is upregulated in cancer samples as compared to normal samples, and means for detecting binding of the antibodies to their epitope associated with tumor cells, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains a radiolabelled antibody preparation for in vivo imaging.

A kit may be designed to detect the level of miRNA renal cancer markers or polynucleotides encoding one or more polynucleotide encoding for protein renal cancer markers in a sample. In an embodiment, the kit includes miRNA renal cancer markers as listed in Table 12 or a precursor thereof or one or more polynucleotides encoding a polypeptide listed in Table 8 that is upregulated in RCC samples as compared to normal samples, or those listed in Table 2 or Table 4 or Table 5 or Table 9, Table 10, or Table 11. Such kits generally include at least one oligonucleotide probe or primer, as described herein, that hybridizes to a polynucleotide encoding one or more renal cancer markers. Such an oligonucleotide may be used, for example, within a PCR or hybridization procedure. Additional components that may be present within the kits include a second oligonucleotide and/or a diagnostic reagent or container to facilitate detection of a polynucleotide encoding one or more renal cancer markers.

The invention provides a kit containing a microarray described herein ready for hybridization to target miRNA renal cancer markers or protein renal cancer polynucleotide markers, plus software for the data analysis of the results. The software to be included with the kit includes data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

The reagents suitable for applying the screening methods of the invention to evaluate compounds may be packaged into convenient kits described herein providing the necessary materials packaged into suitable containers.

The invention contemplates a kit for assessing the presence of renal disease cells, wherein the kit includes antibodies specific for one or more protein renal cancer markers, miRNA renal cancer markers, or primers or probes for polynucleotides encoding the renal cancer markers or miRNA renal cancer markers, and optionally probes, primers or antibodies specific for other markers associated with a renal disease (e.g., cancer).

The invention relates to a kit for assessing the suitability of each of a plurality of test compounds for inhibiting a renal disease (e.g., cancer) in a patient. The kit includes reagents for assessing one or more miRNA renal cancer markers or protein renal cancer markers, miRNA renal cancer markers or polynucleotides encoding the protein renal cancer markers, and optionally a plurality of test agents or compounds.

Additionally the invention provides a kit for assessing the potential of a test compound to contribute to a renal disease (e.g., cancer). The kit includes renal diseased cells (e.g., cancer cells) and reagents for assessing one or more renal cancer markers, miRNA renal cancer markers, polynucleotide encoding for the renal cancer markers and optionally other markers associated with a renal disease.

Therapeutic Applications. One or more renal cancer markers may be targets for immunotherapy. Immunotherapeutic methods include the use of antibody therapy, in vivo vaccines, and ex vivo immunotherapy approaches.

In one aspect, the invention provides one or more renal disease marker antibodies that may be used systemically to treat a renal disease associated with the marker. In particular, the renal disease is renal cancer and one or more renal disease marker antibodies may be used systemically to treat renal cancer. Preferably antibodies are used that target the tumor cells but not the surrounding non-tumor cells and tissue. In a particular embodiment, the renal cancer is RCC. In another embodiment, the renal cancer is clear-cell RCC.

Thus, the invention provides a method of treating a patient susceptible to, or having a disease (e.g., cancer) that expresses one or more renal disease marker, in particular, a marker up-regulated in renal cancer (for example, an up-regulated marker in Table 8 or Table 9, or that of Table 2 and/or Table 4 and/or Table 5 and/or Table 6 or that of Table 7 or Table 12), including administering to the patient an effective amount of an antibody that binds specifically to one or more renal disease marker.

In another aspect, the invention provides a method of inhibiting the growth of tumor cells expressing one or more renal cancer markers, including administering to a patient an antibody which binds specifically to one or more renal cancer markers in an amount effective to inhibit growth of the tumor cells.

One or more renal disease marker antibodies may also be used in a method for selectively inhibiting the growth of, or killing a cell expressing one or more renal disease marker (e.g., tumor cell expressing one or more renal cancer marker) including reacting one or more renal disease marker antibody immunoconjugate or immunotoxin with the cell in an amount sufficient to inhibit the growth of, or kill the cell.

By way of example, unconjugated antibodies to renal cancer markers may be introduced into a patient such that the antibodies bind to renal cancer marker expressing cancer cells and mediate growth inhibition of such cells (including the destruction thereof), and the tumor, by mechanisms which may include complement-mediated cytolysis, antibody-dependent cellular cytotoxicity, altering the physiologic function of one or more renal cancer markers, and/or the inhibition of ligand binding or signal transduction pathways. In addition to unconjugated antibodies to renal cancer markers, one or more renal cancer marker antibodies conjugated to therapeutic agents (e.g., immunoconjugates) may also be used therapeutically to deliver the agent directly to one or more renal cancer marker expressing tumor cells and thereby destroy the tumor. Examples of such agents include abrin, ricin A, Pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1, interleukin-2, interleukin-6, granulocyte macrophage colony stimulating factor, granulocyte colony stimulating factor, or other growth factors.

Cancer immunotherapy using one or more renal cancer marker antibodies may utilize the various approaches that have been successfully employed for cancers, including but not limited to colon cancer (Arlen et al., 1998, Crit Rev Immunol 18: 133-138), multiple myeloma (Ozaki et al., 1997, Blood 90: 3179-3186; Tsunenati et al., 1997, Blood 90: 2437-2444), gastric cancer (Kasprzyk et al., 1992, Cancer Res 52: 2771-2776), B-cell lymphoma (Funakoshi et al., 1996, J. Immunther Emphasis Tumor Immunol 19: 93-101), leukemia (Zhong et al., 1996, Leuk Res 20: 581-589), colorectal cancer (Moun et al., 1994, Cancer Res 54: 6160-6166); Velders et al., 1995, Cancer Res 55: 4398-4403), and breast cancer (Shepard et al., 1991, J. Clin Immunol 11: 117-127).

In the practice of a method of the invention, renal cancer marker antibodies capable of inhibiting the growth of precancer or cancer cells expressing renal cancer markers are administered in a therapeutically effective amount to cancer patients whose lesions or tumors express or overexpress one or more renal cancer markers. The invention may provide a specific, effective and long-needed treatment for renal cancer. The antibody therapy methods of the invention may be combined with other therapies including chemotherapy and radiation.

Patients may be evaluated for the presence and level of expression or overexpression of one or more renal cancer markers in diseased cells and tissues (e.g., tumors), in particular using immunohistochemical assessments of tissue, quantitative imaging as described herein, or other techniques capable of reliably indicating the presence and degree of expression of one or more renal disease markers. Immunohistochemical analysis of tumor biopsies or surgical specimens may be employed for this purpose.

Renal disease marker antibodies useful in treating disease (e.g., cancer) include those that are capable of initiating a potent immune response against the disease (e.g., tumor) and those that are capable of direct cytotoxicity. In this regard, renal disease marker antibodies may elicit cell lysis by either complement-mediated or antibody-dependent cell cytotoxicity (ADCC) mechanisms, both of which require an intact Fc portion of the immunoglobulin molecule for interaction with effector cell Fc receptor sites or complement proteins.

Renal disease marker antibodies that exert a direct biological effect on tumor growth may also be useful in the practice of the invention. Such antibodies may not require the complete immunoglobulin to exert the effect. Potential mechanisms by which such directly cytotoxic antibodies may act include inhibition of cell growth, modulation of cellular differentiation, modulation of tumor angiogenesis factor profiles, and the induction of apoptosis. The mechanism by which a particular antibody exerts an anti-tumor effect may be evaluated using any number of in vitro assays designed to determine ADCC, antibody-dependent macrophage-mediated cytotoxicity (ADMMC), complement-mediated cell lysis, and others known in the art.

The anti-tumor activity of a particular renal cancer marker antibody, or combination of renal cancer marker antibodies, may be evaluated in vivo using a suitable animal model. Xenogenic cancer models, where human cancer explants or passaged xenograft tissues are introduced into immune compromised animals, such as nude or SCID mice, may be employed.

The methods of the invention contemplate the administration of single renal disease marker antibodies as well as combinations, or “cocktails”, of different individual antibodies such as those recognizing different epitopes of other markers. Such cocktails may have certain advantages in as much as they contain antibodies that bind to different epitopes of renal cancer markers and/or exploit different effector mechanisms or combine directly cytotoxic antibodies with antibodies that rely on immune effector functionality. Such antibodies in combination may exhibit synergistic therapeutic effects. In addition, the administration of one or more renal disease marker specific antibodies may be combined with other therapeutic agents, including but not limited to chemotherapeutic agents, androgen-blockers, and immune modulators (e.g., IL2, GM-CSF). The renal disease marker specific antibodies may be administered in their “naked” or unconjugated form, or may have therapeutic agents conjugated to them.

The renal disease marker specific antibodies used in the methods of the invention may be formulated into pharmaceutical compositions including a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences, 16th ed., A. Osal., ed., 1980).

One or more renal disease marker specific antibody formulations may be administered via any route capable of delivering the antibodies to a disease (e.g., tumor) site. Routes of administration include, but are not limited to, intravenous, intraperitoneal, intramuscular, intratumor, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of disease and the severity, grade, or stage of the disease, the binding affinity and half life of the antibodies used, the degree of renal disease marker expression in the patient, the extent of circulating renal disease markers, the desired steady-state antibody concentration level, frequency of treatment, and the influence of any chemotherapeutic agents used in combination with the treatment method of the invention. Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve disease inhibition or regression. Direct administration of one or more renal disease marker antibodies is also possible and may have advantages in certain situations.

Patients may be evaluated for serum cancer markers in order to assist in the determination of the most effective dosing regimen and related factors. The renal cancer assay methods described herein, or similar assays, may be used for quantifying circulating renal disease marker levels in patients prior to treatment. Such assays may also be used for monitoring throughout therapy, and may be useful to gauge therapeutic success in combination with evaluating other parameters such as serum levels of renal cancer markers.

The invention further provides vaccines formulated to contain one or more renal disease marker or fragment thereof.

In an embodiment, the invention provides a method of vaccinating an individual against one or more renal disease marker listed in Table 8 or Table 12, including the step of inoculating the individual with the marker or fragment thereof that lacks activity, wherein the inoculation elicits an immune response in the individual thereby vaccinating the individual against the marker.

The use in anti-cancer therapy of a tumor antigen in a vaccine for generating humoral and cell-mediated immunity is well known and, for example, has been employed in prostate cancer using human PSMA and rodent PAP immunogens (Hodge et al., 1995, Int. J. Cancer 63: 231-237; and Fong et al., 1997, J. Immunol. 159: 3113-3117). These and similar methods can be practiced by employing one or more renal cancer markers, or fragment thereof, or polynucleotide encoding for the markers and recombinant vectors capable of expressing and appropriately presenting renal disease marker immunogens.

By way of example, viral gene delivery systems may be used to deliver one or more protein renal cancer polynucleotide markers or miRNA renal cancer markers or complementary anti-sense versions thereof. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding one or more renal cancer marker or fragment thereof introduced into the patient (e.g., intramuscularly) to induce an anti-tumor response.

Various ex vivo strategies may also be employed. One approach involves the use of cells to present one or more renal disease marker to a patient's immune system. For example, autologous dendritic cells which express MHC class I and II, may be pulsed with one or more renal disease marker or peptides thereof that are capable of binding to MHC molecules, to thereby stimulate the patients' immune systems (see, for example, Tjoa et al., 1996, Prostate 28: 65-69; Murphy et al., 1996, Prostate 29: 371-380).

Anti-idiotypic renal disease marker specific antibodies can also be used in therapy as a vaccine for inducing an immune response to cells expressing one or more renal disease marker. The generation of anti-idiotypic antibodies is well known in the art and can readily be adapted to generate anti-idiotypic renal cancer marker specific antibodies that mimic an epitope on one or more renal cancer markers (see, for example, Wagner et al., 1997, Hybridoma 16: 33-40; Foon et al., 1995, J. Clin Invest 96: 334-342; and Herlyn et al., 1996, Cancer Immunol Immunother 43: 65-76). Such an antibody can be used in anti-idiotypic therapy as presently practiced with other anti-idiotypic antibodies directed against antigens associated with disease (e.g., tumor antigens).

Genetic immunization methods may be utilized to generate prophylactic or therapeutic humoral and cellular immune responses directed against cells expressing one or more renal cancer marker. One or more DNA molecules encoding renal cancer markers, constructs including DNA encoding one or more renal cancer markers/immunogens and appropriate regulatory sequences may be injected directly into muscle or skin of an individual, such that the cells of the muscle or skin take-up the construct and express the encoded renal cancer markers/immunogens. The renal cancer markers/immunogens may be expressed as cell surface proteins or be secreted. Expression of one or more renal cancer markers results in the generation of prophylactic or therapeutic humoral and cellular immunity against the disease (e.g., cancer). Various prophylactic and therapeutic genetic immunization techniques known in the art may be used.

The invention further provides methods for inhibiting cellular activity (e.g., cell proliferation, activation, or propagation) of a cell expressing one or more renal disease marker. This method includes reacting immunoconjugates of the invention (e.g., a heterogeneous or homogenous mixture) with the cell so that renal cancer markers form complexes with the immunoconjugates. A subject with a neoplastic or preneoplastic condition can be treated when the inhibition of cellular activity results in cell death.

In another aspect, the invention provides methods for selectively inhibiting a cell expressing one or more renal disease marker by reacting any one or a combination of the immunoconjugates of the invention with the cell in an amount sufficient to inhibit the cell. Amounts include those that are sufficient to kill the cell or sufficient to inhibit cell growth or proliferation.

Vectors derived from retroviruses, adenovirus, herpes or vaccinia viruses, or from various bacterial plasmids, may be used to deliver polynucleotides encoding renal cancer markers or miRNA renal cancer markers to a targeted organ, tissue, or cell population. Methods well known to those skilled in the art may be used to construct recombinant vectors that will express antisense miRNA renal cancer markers or polynucleotides for renal cancer markers. (See, for example, the techniques described in Sambrook et al. (supra) and Ausubel et al. (supra).)

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

Genes encoding renal cancer markers can be turned off by transfecting a cell or tissue with vectors that express high levels of a desired renal disease marker-encoding fragment. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of a gene encoding a renal disease marker, i.e., the promoters, enhancers, and introns. Preferably, oligonucleotides are derived from the transcription initiation site, (e.g., between −10 and +10 regions of the leader sequence). The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al., in: Huber and Carr (1994), Molecular and Immunologic Approaches, Futura Publishing Co, Mt Kisco, N.Y.

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a renal disease marker.

Specific ribozyme cleavage sites within any potential RNA target may initially be identified by scanning the target molecule for ribozyme cleavage sites which include the following sequences, GUA, GUU, and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

One or more renal cancer markers and polynucleotides encoding the markers, and fragments thereof, may be used in the treatment of a renal disease (e.g., cancer) in a subject. In an aspect the renal cancer markers and polynucleotides encoding the markers are renal cancer markers that are down-regulated in renal cancer, for example, F2, PTMA and one or more of the down-regulated markers listed in Table 8. In another aspect, the renal cancer markers are miRNA renal cancer markers that are down-regulated in renal cancer and one or more of the down-regulated markers listed in Table 9. The markers or polynucleotides may be formulated into compositions for administration to subjects suffering from a renal disease. Therefore, the present invention also relates to a composition including one or more renal disease cancer markers or polynucleotides encoding the markers, or a fragment thereof, and a pharmaceutically acceptable carrier, excipient or diluent. A method for treating or preventing a renal disease in a subject is also provided including administering to a patient in need thereof, one or more renal cancer markers or polynucleotides encoding the markers, or a composition of the invention.

The invention further provides a method of inhibiting a renal disease (e.g., cancer) in a patient by: (a) obtaining a sample including diseased cells from the patient; (b) separately maintaining aliquots of the sample in the presence of a plurality of test agents; (c) comparing levels of one or more renal cancer markers, and/or polynucleotides encoding one or more renal cancer markers in each aliquot; and (d) administering to the patient at least one of the test agents which alters the levels of the renal cancer markers, markers, and/or polynucleotides encoding one or more cancer markers in the aliquot containing that test agent, relative to the other test agents.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA, 1985). On this basis, the compositions include, albeit not exclusively, the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment. The compositions of the invention may be administered concurrently, separately, or sequentially with other therapeutic agents or therapies. The therapeutic activity of compositions, agents, and compounds may be identified using a method of the invention and may be evaluated in vivo using a suitable animal model.

The inventors' study is a significant advancement in this direction as it lays major thrust on determining the clinical impact of a proteomics and miRNA based biomarker in diagnosing tumor in patients showing symptoms of renal disease.

The present invention is described in the following non-limiting Examples, which are set forth to illustrate and to aid in an understanding of the invention, and should not be construed to limit in any way the scope of the invention as defined in the claims which follow thereafter.

### EXAMPLES

In Examples 1-7, the inventors demonstrate the identification of a consistently increased expression of a panel of proteins, including YWHAH, CAPNS1, KNG1, and/or SERPING1, that serve as renal cancer biomarkers. In Examples 8-12, the inventors demonstrate the identification of miRNAs that are selectively overexpressed in renal cancer tissues.

### Example 1

### Samples and Reagents

Tumor tissue from patients diagnosed with RCC and their adjacent normal counterparts were obtained from nephrectomy specimens at St. Michael's hospital, after obtaining an informed consent, or from the Ontario Tumor Bank. As RCC is known to arise from the proximal tubules (Pavlovich, Schmidt 2004), the kidney cortex was used as a normal control (Sarto et al., 1997; Shi et al., 2004). The histologic diagnosis for each sample was reconfirmed using microscopic examination of a hematoxylin-and-eosin-stained frozen section of each research tissue block. The tissue from the mirror face of the histologic section was then washed three times in approximately 1 ml of phosphate-buffered saline (PBS) with a cocktail of protease inhibitors, as described previously (1 mM 4-(2-aminoethyl) benzenesulfonyl fluoride, 10 μM leupeptin, 1 μg/ml aprotinin, and 1 μM pepstatin) (1). The washed tissue was then homogenized in 0.5 ml PBS with protease inhibitors, using a handheld homogenizer. These homogenates were then flash-frozen in liquid nitrogen and stored at −80° C. until use. Samples were thawed and clarified by centrifugation, and the protein concentration was determined by a Bradford-type assay using Bio-Rad protein quantification reagent (Bio-Rad, Mississauga, ON, Canada).

The inventors utilized a tissue preparation protocol that was previously used for biomarker studies in endometrial cancer (1 DeSouza et al., 2005a; 7DeSouza et al., 2007). In summary, cell debris from the tissue homogenates was removed by centrifugation in a microfuge at 4° C. for 30 min at 14 000 rpm. The clarified supernatant was transferred to fresh microfuge tubes, and the total protein content was determined using a commercial Bradford assay reagent (Bio-Rad, Mississauga, ON, Canada). A standard curve for the Bradford assay was made using γ-globulin as a control. 100 μg of each sample were then denatured, and the cysteines were blocked as described in the iTRAQ protocol (Applied Biosystems, Foster City, Calif.). Each sample was then digested with trypsin, as recommended in the iTRAQ protocol, and labeled with the iTRAQ tags as follows: non-cancer, diseased kidney, iTRAQ114; normal kidney, iTRAQ115; and the two kidney cancer samples, iTRAQ116 and iTRAQ117. The labeled samples were then pooled and mixed with Eluent A (10 mM KH2PO4 solution in 25% acetonitrile and 75% deionized water acidified to a pH of 3.0 with phosphoric acid) to a total volume of 1.0 mL for strong cation exchange (SCX) chromatography. This diluted sample was further acidified using 5 μL of concentrated phosphoric acid, after which the contents were manually injected onto a 200 μL bed-volume strong cation exchange (SCX) cartridge (Applied Biosystems Inc., Foster City, Calif.). Separation was effected first by a wash with 1.0 mL of Eluent A, and then by ten step-elutions using 0.5 mL each of Eluent A with increasing concentrations of KCl. The ten salt concentrations used were 10 mM, 50 mM, 100 mM, 150 mM, 200 mM, 250 mM, 300 mM, 350 mM, 500 mM, and 1M KCl. Following fractionation, the samples were dried by speed-vacuuming and stored at −20° C. Prior to reverse-phase nanobore liquid chromatography—tandem mass spectrometric (nanoLC MS/MS) analysis, these fractions were redissolved in 10 μL of an aqueous solution of 1.0% formic acid.

### Example 2

### Strong Cation Exchange (SCX) Separation Conditions

For the offline 2D LC-MS/MS analysis, each set of labelled samples was first separated by SCX fractionation using an HP1050 high-performance liquid chromatograph (Agilent, Palo Alto, Calif., U.S.) with a 2.1-mm internal diameter (ID)×100-mm length polysulfoethyl A column packed with 5-μm beads with 300 Å pores (The Nest Group, Southborough, Mass.), as previously described (21). A 2.1-mm ID×10-mm length guard column of the same material was fitted immediately upstream of the analytical column. Separation was performed, as previously described (21). Briefly, each pooled sample set was diluted with the loading buffer (15 mM KH₂PO₄ in 25% acetonitrile, pH 3.0) to a total volume of 2 ml and the pH adjusted to 3.0 with phosphoric acid. Samples were then filtered using a 0.45-μm syringe filter (Millipore, Cambridge, ON, Canada) before loading onto the column. Separation was performed using a linear binary gradient over one hour. Buffer A was identical in composition to the loading buffer, while Buffer B was Buffer A containing 350 mM KCl. Fractions were collected every two minutes using an SF-2120 Super Fraction Collector (Advantec MFs, Dublin, Calif.), after an initial wait of 2 minutes to accommodate the void volume. This resulted in a total of 30 SCX fractions per sample set. These fractions were dried by speed vacuuming (Thermo Savant SC110 A, Holbrook, N.Y.) and resuspended in 30 μl of 0.1% formic acid each.

For the online 2D LC-MS/MS analysis, an SCX cartridge (BioX-SCX, LC Packings, The Netherlands) was plumbed upstream of the reverse phase (RP) desalting cartridge and analytical column. This SCX cartridge was connected through a second valve on the Switchos unit. Samples were separated on this SCX cartridge using 10 μl step elutions with increasing concentration of ammonium acetate (10 mM, 50 mM, 100 mM, 150 mM, 200 mM, 250 mM, 300 mM, 350 mM, 500 mM and 1M). The flow path used for these steps was designed to ensure that there was never any flow reversal through either of the cartridges (SCX or RP). Separation on the RP analytical column was effected as described for the second stage of the offline LC-MS/MS analysis described below.

### Example 3

### LC-MS/MS Run Conditions

The inventors used a nanobore LC system from LC Packings (Amsterdam, The Netherlands) consisting of a Famos autosampler and an Ultimate Nano LC system. The LC system was interfaced to an API QSTAR Pulsar-i hybrid quadrupole/time-of-flight (QqTOF) tandem mass spectrometer (Applied Biosystems/MDS Sciex, Foster City, Calif.) equipped with a Protana NanoES ion source (Protana Engineering NS, Odense, Denmark). The spray capillary was a PicoTip SilicaTip emitter with a 10-μm ID tip (New Objective, Woburn, Mass.). The nanobore LC column was 75-μm ID×150-mm length reverse-phase nano capillary column packed in-house with 3 μm C18 beads with 100 Å pores (Kromasil). One μL of sample was injected via the “μL-pick-up” mode. Separation was performed using a binary mobile-phase gradient at a total flow rate of 200 mL/min. For nanospray analysis, the following source conditions were used: a curtain-gas setting of 20 and an ionspray voltage of 1800-3000 V, Q0 declustering potential of 65 V, and a focusing potential 265 V. Nitrogen was used as the collision gas (CAD setting=5) for both TOF MS and MS/MS scans. All nanoLC MS/MS data were acquired in information dependent acquisition (IDA) mode using Analyst QS 1.1 (Applied Biosystems/MDS SCIEX). The inventors performed two sets of analysis for each fraction. MS cycles consisted of a TOF MS survey scan with an m/z range of 400-1500 Th for 1 s, followed by five product ion scans with an m/z range of 80-2000 Th for 2 s each. Collision energy (CE) was automatically controlled by the IDA CE Parameters script. Switching criteria were set to ions greater than 400 Th and smaller than 1500 Th with a charge state of 2 to 5 and an abundance of ≧10 counts/s. Former target ions were excluded for 30 s and ions within a 4-Th window were ignored. In addition, the IDA Extensions II script was set to no repetitions before dynamic exclusion. In all experiments, the script was set to select a precursor ion nearest to a threshold of 15 count/s every 4 cycles. These settings ensured examination of not only high abundance ions, but low abundance ones as well. FIG. 1 shows a representative scan.

### Example 4

### Data Analysis

Data analysis for the iTRAQ experiments were performed with ProteinPilot version 2.0.1 (Applied Biosystems) using a human Celera protein sequence database (human KBMS 20041109) provided by Applied Biosystems that contained a total of 178,239 protein sequences and included sequences from NCBI's nr, refseq, SwissProt, TrEMBL and Celera databases. ProteinPilot utilizes the Paragon algorithm for assigning sequence identity (Shilov et al. 2007). Redundancy between proteins identified is minimized using a grouping function, which assigns an “unused score” to the peptides that are unique to a protein or group of redundant proteins. The cut-off unused score used for assessing detection was 1.3, which corresponds to a confidence of 95%. Relative quantification of proteins in the case of iTRAQ is performed on the MS/MS scans and is the ratio of the areas under the peaks at 114, 115, 116, and 117 Da which are the masses of the tags that correspond to the iTRAQ reagents. The protein ratios are calculated using the individual ratios of the peptides with a weighting factor incorporated based on the confidence of the matching peptide. Normalization of the ratio is performed by the ProteinPilot using the median ratio obtained across all the proteins identified in a run. This normalization is based on the assumption that most proteins will not show significant differential expression between the samples. The normalization factor is termed the ‘Applied bias’ and is calculated for each pair of samples.

### Example 5

### Bioinformatics Analysis

Proteins were classified into groups according to subcellular compartmentalization (e.g., cytoplasm, nucleus, membranes, etc.), biological process (e.g., cell cycle proteins), and molecular function (e.g., receptor binding). These analyses were performed through the Gene Ontology (GO) Consortium databases (http://www.geneontology.org/) and their related analytical tools. Bioinformatic validation of gene expression in normal and cancerous tissue was performed via the SwissProt Protein knowledgebase and the TrEMBL annotated databases (http://expasy.org/sprot/), GeneCards (http://www.genecards.org/), and NCBI (http://www.ncbi.nlm.nih.gov/). In silico analysis and validation of differential gene expression in cancer were carried out via two independent databases: the SAGE database of the Cancer Genome Anatomy Project (CGAP) (http://cgap.nci.nih.gov/), and the UniGene EST ProfileViewer (EPV), Digital Gene Expression Displayer (DGED), and xProfiler analysis (http://www.ncbi.nlm.nih.gov/). EPV expression values are calculated as transcript/million. DGED values are calculated as the sequence odds ratios. A pool of 5 normal kidney libraries and 7 kidney cancer libraries (RCC) were chosen for the DGED analysis. For SAGE, relative expression values were used for analyses (defined as tags/200,000). Specimens from both tissues and cell lines were incorporated into the analyses. Tags that map to more than one gene and those that are not informative (no expression values in either cancer or normal) were excluded; cases with discrepancy between different tags were also excluded.

### Example 6

### Verification of Candidate Potential Cancer Markers (PCMS) by Immunohistochemistry

Paraffin blocks were sectioned 4 μm thick, mounted on slides, and dried overnight. Sections were deparaffinized in xylene and rehydrated through decreasing graded alcohols. Slides were immunostained using the Benchmark® XT (Ventana, Tucson, Ariz.) with monoclonal antibodies for vimentin (Ventana, Tucson, Ariz.) and phospho-S6 ribosomal protein (Cell Signaling Tech, Danver, Mass.). Immune complex was visualized by incubating with diaminobenzidine (DAB), and sections are counterstained with hematoxylin. Five pairs of normal and cancer from the same patient were examined. Slides were reviewed and scored (% positivity and density) independently by two pathologists.

### Example 7

### Dot Blot Analysis of Proteins in RCC and Normal Tissues

Fresh frozen tissue samples that were stored in −80° C. were used for protein validation experiments. Frozen tissues were weighted and suspended in cold 1×PBS buffer with protease inhibitors (Roche Diagnostics, Indianapolis, Ind.) to a concentration of 2.5% (w/v). Samples were homogenized with a hand-held tissue homogenizer for 30 sec on ice and subsequently centrifuged for 1 min at 8,000 rpm. The supernatant of the homogenate was used for the dot blot experiments.

Five pairs of coupled normal can cancer tissues were tested by immunoblotting. 2 μL of tissue homogenate for each sample was added to labeled nitrocellulose membrane and dried in a warming chamber for 1 hour. The membrane was then blocked with TBS-T buffer (pH 7.5) with 5% milk for 1 hour at RT. The membrane was probed with monoclonal antibodies for NNMT, LDHAL6B and SERPING1 (Cedarlane Labs Canada, Burlington, ON) diluted in TBS-T buffer as per manufacturer's recommendation. The membranes were washed 3 times in TBS-T buffer and probe with HRP conjugated anitmouse antibody (Fisher Scientific Canada, Ottawa, ON) for 30 min in RT. The membranes were washed 3 times in TBS-T buffer and once in TBS buffer. 3 mL of chemiluminescent reagent (Diagnostic Product Corporation, Gwynedd, UK) was added to each membrane and the membranes were incubated for 3 min. The membranes were then exposed to x-ray film for 30 min.

Provided below is a summary of the results obtained by the inventors in connection with the experiments of Examples 1-7:

The inventors identified a total of 937 proteins in both runs. Using cutoff values of 1.5 fold for overexpression, and 0.67 fold for underexpression, the inventors were able to identify 168 underexpressed proteins, and 156 proteins that were overexpressed in RCC compared to their normal counterparts. These cutoff values were used by the inventors for selecting proteins for further statistical analyses in previous studies (Ralhan et al. 2008) and were found to perform satisfactorily. Table 1 shows the distribution of over- and underexpressed proteins in RCC specimens in the two runs. There were 65 proteins that were recognized in both runs as being underexpressed, while 34 proteins as being overexpressed. There was a statistically significant positive correlation of normalized protein levels identified in both runs (rp=0.695, p<0.001) (FIG. 2). Cellular localization of the inventors' extracted proteins was determined using the GO analysis and most proteins were assigned to cellular compartments: 34% of the proteins were cytoplasmic, 13% nuclear, 11% were localized to the mitochondria, 4% membranous, and 6% were extracellular proteins (FIG. 3A). A comparable distribution was seen for the overexpressed proteins (FIG. 3B). Proteins of cytoplasmic and membranous distribution are of particular importance, as they may have potential for use as tumor markers in biological fluids. Table 8 shows a list of the dysregulated proteins in RCC compared to their normal counterparts, along with their chromosomal locations: 49 proteins demonstrated an average increase of ≧2.0 fold; nine proteins showed ≧3.0 fold average increase in expression levels. Underexpressed proteins ranged from 0.04-0.67 in expression relative to normal tissues.

In order to examine if these dysregulations were specific to RCC, or represented a nonspecific response by the kidney cells, the inventors analyzed two control specimens simultaneously: a transitional cell carcinoma (TCC, with a distinct origin from the transitional urothelium of the kidney) and kidney tissue from a case of end-stage glomerulonephritis. Protein expressions were normalized using expression levels in normal kidney tissue, and expressed as fold changes of normal. The normalized average expression in RCC was lower than normalized non-cancerous renal failure in 454 proteins and higher in 483 proteins (p=0.020) (FIG. 4A). RCC normalized average expression values were also lower than those of normalized TCC in 415 proteins, and higher in 522 proteins (p=0.005) (FIG. 4B). Table 3 shows the relative normalized protein expression ratios between RCC, renal failure and TCC. The inventors also compared the inventors' results with four previously published reports of differential protein expression in kidney cancer (Craven et al. 2006; Perego et al. 2005; Sarto et al. 1997; Shi et al. 2004). There are 24 proteins from the inventors' list of proteins that were identified in the other reports; 15 of them were shown to be differentially expressed by one report, five identified by two additional reports, and four recognized by three studies (Table 4).

The inventors performed an in silico validation of the inventors' results by different approaches. In addition, the inventors did a thorough literature search for individual up- and downregulated proteins: a few of the inventors' dysregulated proteins were found to be previously reported as tumor markers for RCC (vide infra). The inventors also performed a database search through the SwissProt Knowledgebase. Table 5 shows a partial list of proteins from the inventors' list with documented expression in the kidney.

To validate the inventors' protein findings and examine whether they are reflected at the mRNA level, the inventors performed SAGE and EST analysis. SAGE data verified overexpression of 65% of all genes with informative expression data (data not shown). When focusing on proteins that were identified in both runs, the SAGE mRNA data were in concordance with the protein results in 84% of cases (Table 6). EST analysis of informative genes using both the EST ProfileViewer and the Digital Gene Expression Displayer search engines confirmed upregulation in cancer compared to normal in 74% of cases by at least one search engine (Table 7).

Immuohistochemistry and dot blot were employed to further validate the protein expression results. Vimentin, which was found to overexpressed in RCC in the inventors'MS/MS analyses, was validated by immunohistochemistry (FIG. 5). Staining for vimentin antibodies was weak or largely absent in normal cancer tissue while significant staining can be observed in RCC tissue. Similar result was obtained using phospho-S6 ribosomal protein (another overexpressed protein) antibodies (Data not shown). Three highly overexpressed proteins were selected for dot blot analysis. (FIG. 6). Dot blot analysis showed all 3 proteins were overexpressed in RCC tissue, further confirming the MS/MS results.

In order to validate the potential clinical utility of the dysregulated proteins as potential serum biomarkers, the inventors performed a literature and bioinformatics search (through public databases) of the inventors' upregulated proteins and were able to identify 23 of these proteins being secreted in the blood (data not shown). The inventors are currently performing individual validation of few of these biomarkers in serum of RCC patients.

Without being bound by theory, the results obtained in the experiments of Examples 1-7 are discussed below:

This study is the first report of quantitative protein analysis of clear-cell RCC utilizing isotope affinity tags (iTRAQ). Using this approach, the inventors identified dysregulated proteins in clear-cell RCC by direct tissue analysis. The robustness of the analysis is confirmed by the good positive correlation between the two runs (rp=0.695, p<0.001) (FIG. 2). Proteomic analyses are directly related to clinical applications, since proteins determine the ultimate phenotypic expression in cancer tissues. Advanced features of the inventors' mass spectrometric analyses include simultaneous profiling of multiple admixed specimens, which helps to eliminate artificial differences due to differential protein losses during purification or separation (8DeSouza et al. 2005b). In addition, the use of four isotopic labels allows quantitative comparison among four different tissue samples. Differences between the inventors' results and other previously published proteomics analyses of RCC (6, 20-22) could be attributed to technical differences (i.e., the mass spectral approaches), and the nature of the material analyzed (e.g., cell culture vs. tissue). Another important factor to consider is that the inventors' protocol is the first to allow “quantitative” analysis of protein expression. Other important factors to be considered are tumor heterogeneity and the differences in histological types of the tumors analyzed.

The inventors have attempted to validate the inventors' results by using multiple bioinformatics approaches. There was 60-70% agreement on average, between the inventors' results and two independent databases (EST and SAGE) (Tables 6-7) analyzed by multiple search engines. Differences and inconsistencies between the inventors' protein analysis and these RNA data could be attributed to the possibility of post-transcriptional modifications and the inherited inaccuracy of SAGE quantification, especially at low expression levels. Other technical issues with EST library construction, like subtraction or normalization, could have also be potential sources of discrepancy. Literature searches revealed that many of the inventors' dysregulated proteins were previously reported as potential tumor markers for kidney cancer. Major vault protein (MVP), also known as lung resistance-related protein (LRP) (Table 2, No. 32), has been documented to be expressed in the normal kidney tubules (23). A recent study showed that this protein is upregulated in RCC (24). Overexpression of LRP predicts a poor response to chemotherapy. In addition to kidney cancer, LRP is also upregulated in ovarian cancer and other malignancies (23, 25). Another gene that is upregulated in clear-cell RCC is the adipose differentiation-related protein (ADFP). ADFP (Table 2, No. 33) is a lipid storage droplet-associated protein, and its transcription is considered to be regulated by the von Hippel-Lindau/hypoxia-inducible factor pathway (26). Patients with higher AFDP levels showed significantly better survival than those who did not (27).

Nicotinamide N-methyltransferase (NNMT) (Table 2, No. 7) was also shown to be upregulated in RCC. Patients with higher NNMT levels showed significantly better survival than those expressing lower levels (26). In addition to RCC, NNMT was also shown to be dysregulated in other malignancies, including stomach adenocarcinoma, glioblastoma, papillary thyroid carcinoma, and oral squamous cell carcinoma (28). NNMT catalyzes the N-methylation of nicotinamide and is involved in xenobiotic metabolism. Nicotinamide is a part of the NAD molecule, which is involved in many important biological processes including cellular resistance and energy production (29). Preliminary evidence suggest that NNMT might have potential therapeutic applications in cancer (28). A recent study has also shown that it might help in predicting response to radiation in bladder cancer (30). Thymidine phosphorylase (TP) (Table 2, No. 31), known as a platelet-derived endothelial cell growth factor (PD-ECGF), is a mitogenic and angiogenic factor derived from platelets (31). TP levels were found to be higher in various types of malignant tumor, including RCC, than the adjacent normeoplastic tissues (32). TP was found to be an unfavorable independent prognostic factor in RCC (32).

Alpha-crystallin β (Table 2, No. 23) is a major protein component of the vertebrate eye lens with chaperone-like functions and is a member of the small heat shock protein HSP20. It has been shown to be dysregulated in RCC (6). Pinder et al. reported that 90% of RCC tissue specimens they studied show strong staining by anti-αβ-crystallin antibody (33). The biological significance of the overexpression of αβ-crystallin in RCC carcinogenesis is unclear. It probably represents a stress response in RCC given the fact that αβ-crystallin is a member of small heat-shock protein HSP20 family. In addition, the inventors identified a number of new biomarkers. For example: Prothymosin alpha (Table 2, No. 81) is a small, highly acidic, nuclear protein that has been proposed to play a role in cell proliferation and immune regulation (34). Prothymosin alpha has recently been proposed to be a potential marker of proliferation in patients with thyroid cancer (35). This protein was implicated in various other cancers, including gastric, lung, liver, colon, breast, and head and -neck cancers (14, 36-40). Another interesting protein is the tryptophan 5-monooxygenase activation protein (Table 2, No. 2), which is a protein kinase-dependent activator of tyrosine and tryptophan hydroxylases, and is an endogenous inhibitor of protein kinase C. This family of proteins mediates signal transduction by binding to phosphoserine-containing protein (41). Poly (rc)-binding protein 2 (Table 2, No. 9), another up regulated protein, is a poly(rc)-binding protein with translational regulatory function (42). CAPNS1 (calpain, small subunit 1) (Table 2, No. 3), belongs to the calcium-dependent cysteine proteinases that has a regulatory function. It was recently reported as a cancer-associated gene (43) that undergoes alternative splicing in cancer tissues leading to removal of the N-terminal glycine-rich domain implicated in interaction with lipids (43). In conclusion, the inventors' quantitative mass spectrometry analysis revealed a number of potential proteins that are dysregulated in RCC. This was verified by in silico analysis and literature review. Whether these proteins are consistently differentially expressed in RCC will require a larger-scale analysis in the future. The inventors are currently pursuing validation of these potential biomarkers with an independent technique and assessment of their clinical utility in RCC diagnosis and prognosis.

### Example 8

### Specimen Collection for miRNA Analysis

Fresh kidney tissues were obtained from patients who underwent nephrectomy for RCC, following the Research Ethics Board approval of St. Michael's Hospital. Seventy specimen pairs in total were dissected from both cancer and adjacent normal kidney cortex tissue from the same patient. The specimens were collected in cryotubes and stored at −80° C. for future use. All diagnoses were histologically verified by a pathologist. For the discovery phase, miRNA microarray analysis was performed on 20 ccRCCs and their and non-malignant counterparts from the same patient. For the validation experiment, the inventors used a separate cohort of 50 matched pairs of RCC and normal kidney tissue.

### Example 9

### Total RNA Extraction

Two mg each of normal kidney and malignant tissue were used for nucleic acid isolation. Total RNA was extracted using miRNeasy (Qiagen, Mississauga, Canada) according to the manufacture's protocol. Total RNA concentration was determined spectrophotometrically at 260 nM (NanoDrop 1000 Spectrophotometer, NanoDrop Technologies Inc., Wilmington, Del.), and the quality of extracted RNA was assessed by electropherogram and gel analysis. Samples suitable for analysis were stored at −80° C.

### Example 10

### miRNA Microarray

Microarray analysis was performed on 5 μg of total RNA from histologically confirmed cancer and adjacent normal tissues from the same patient, and was carried out using the μParaflo® microfluidic technology, as per the manufacturer's protocol (LC Sciences, Houston, Tex.). Hybridization was performed overnight on a microfluidic chip. On the microfluidic chip, each detection probe consisted of a chemically modified nucleotide coding segment complementary to target microRNA and a spacer segment of polyethylene glycol to extend the coding segment away from the substrate. The array covers all miRNA transcripts available in the latest version of the Sanger miRBase database (Release 13.0). Post-hybridization detection used fluorescence labelling with tag-specific Cy3 and Cy5 dyes. Hybridization images were collected using a GenePix 4000B laser scanner (Molecular Device, Sunnyvale, Calif.) and digitized using Array-Pro image analysis software (Media Cybernetics, Bethesda, Md.). The data was analyzed by first subtracting the background and then normalizing the signals to balance the intensities of the Cy3 and Cy5 labelled transcripts so that differential expression ratios can be calculated. The ratio of the two sets of detected signals [log 2 (cancer/normal)] and p values of the t test were calculated; differentially detected signals were those with a p value less than 0.05. Heat maps were generated for the differentially expressed miRNAs at the detectable signal.

### Example 11

### Quantitative RT-PCR

Quantitative miRNA RT-PCR was performed with the TaqMan microRNA Assay® kit using the supplier's protocol (Applied Biosystems, Foster City, Calif.). The miRNA transcripts of five of the top 35 dysregulated miRNAs (miR-34a, miR-155, miR-200c, miR-210, and miR-1974) were first reverse-transcribed into cDNA using gene-specific miRNA qRT-PCR primer sets. This was followed by real-time PCR amplification for 40 cycles using Step One™ Plus Real-Time PCR System and miRNA specific probes (Applied Biosystems, Foster City, Calif.). Experiments were performed in duplicate. Expression values were normalized to a small nucleolar RNA, RNU44 (Applied Biosystems, Foster City, Calif.) which has been proven to have consistent expression levels in malignant and non-malignant tissue pairs (9a). Fifty pairs of kidney tumours and their adjacent normal kidney tissue were used for the RT-PCR analysis. ΔCt values were calculated using the ΔCt values of the normal tissue and the cancerous tissue for each miRNA probe.

### Example 12

### Bioinformatics Analysis

The inventors used several programs to perform bioinformatics-based target prediction analysis. MiRNA target prediction analyses were performed using TargetCombo (http://diana.pcbi.upenn.edu/cgi-bin/TargetCombo.cgi) and selecting the option Predicted Targets: Union. This option searches all targets predicted by any of the four prediction programs, including DIANA-microT, PicTar, TargetScanS, and miRanda. The inventors also used miRecords (http://mirecords.biolead.org), which compiles miRNA target predictions from 11 different algorithms: miRanda, PicTar, TargetScan Version 4.1, DIANA-microT, Microlnspector, MirTarget2, miTarget, NBmiRTar, PITA, RNA22, and RNAhybrid. A positive prediction was only included if it was detected by at least four programs. In silico analysis of miRNA expression in different malignancies was done by compiling a database of published dysregulated miRNAs in different cancer types (which included all publicly available published data at the time of analysis) (10a-32a). This was used to compare the expression of miRNAs dysregulated in kidney cancer with other malignancies.

Chromosomal locations of miRNAs were determined using miRBase database (Release 14) (http://www.mirbase.org/). The inventors compared the chromosomal locations of dysregulated miRNAs in ccRCC to aberrations previously reported in kidney cancer by comparative genomic hybridization (CGH) analysis and loss of heterozygosity (LOH) using the Progenetix molecular cytogenetic database (http://www.progenetix.net/progenetix/index.html), where average genetic changes from 835 cases of kidney cancer were reported. Chromosomal aberrations were defined as either gain or loss of chromosomal material.

Provided below is a summary of the results obtained by the inventors in connection with the experiments of Examples 8-12:

miRNA microarray. The inventors performed microarray analysis on twenty pairs of renal cell carcinoma and normal adjacent kidney tissue. As summarized in Table 9 and Table 12, the inventors identified a total of 166 differentially expressed miRNAs in clear-cell RCC compared to normal. Eighty-nine miRNAs had increased expression, while 77 had decreased expression in ccRCC. The top 35 dysregulated miRNAs in ccRCC are shown in Table 9. FIG. 8 shows a representative heat map of the statistically significant (p<0.05) results.

Experimental validation. The inventors experimentally verified expression levels of five of the top 35 dysregulated miRNAs—miR-34a, miR-155, miR-200c, miR-210, and miR-1974—on a separate cohort of fifty matched RCC and normal tissues, with miRNA-specific probes using qRT-PCR analysis. As seen in FIG. 9, the log 2 fold change values of the 5 miRNAs are comparable to the microarray results in Table 9.

Expression of RCC dysregulated miRNAs in other malignancies. The inventors compared their results to experimental results from other published studies of miRNA differential expression and its role in the pathogenesis of other malignancies (10a, 36a-57a). Twenty-one of the 35 top dysregulated miRNAs identified in the inventors' study have been shown to play roles in other malignancies, and their involvement in the pathogenesis of cancer has been examined. A partial list, complete with miRNA target proteins, is presented in Table 10.

By way of example, some miRNAs that were found to be involved in other malignancies include miR-21, miR-155, and the miR-200 family, just to name a few. MiR-21 has been reported to be dysregulated in colon, gastric, and breast cancers and in glioblastomas; it has also been shown to be involved in cell survival and cellular migration and invasion through targeting proteins such as PTEN and PCDC4. The miR-200 family (miR-200a, miR-200b, miR-200c, miR-141, and miR-429) was significantly downregulated in RCC when compared to normal kidney tissue. This family has also been shown to inhibit epithelial to mesenchymal transition and to target the repressors of e-cadherin ZEB1 and ZEB2. Their decreased expression in malignancy contributes to an increase in the aggressiveness of cancer cells. Aberrant expression of miR-155 has been reported in breast and pancreatic cancer as well as lymphoma. Mir-155 has also been shown to be capable of targeting FOXO3 and RHOA1.

Target prediction analysis. In order to understand the potential role of miRNAs in RCC pathogenesis, the inventors identified potential targets of the 166 dysregulated miRNAs using target prediction analysis as described in the Examples. As shown in FIG. 10, a number of proteins that are documented to be involved in RCC pathogenesis are predicted to be targeted by miRNAs. The direction of dysregulation of miRNAs presented in the figure agreed with the target protein dysregulation in ccRCC—i.e., miRNAs upregulated in ccRCC target proteins that are downregulated in kidney cancer pathogenesis. Most notable is the potential interaction of dysregulated miRNAs with the von Hippel-Lindau (VHL) protein. In total, the inventors identified ten miRNAs significantly upregulated in ccRCC that can potentially target VHL.

Chromosomal distribution of RCC dysregulated miRNAs. The chromosomal locations of the 166 differentially expressed miRNAs were mapped according to Release 13.0 of the miRBAse database (59a) and compared to databases of chromosomal aberrations in kidney cancer (see Examples). Of the 166 significantly dysregulated miRNAs in ccRCC, the inventors identified 75 (45%) miRNAs with dysregulation patterns that agreed with chromosomal gains and losses reported for ccRCC. Most of the dysregulated miRNAs mapped to chromosomes 1, 9, 17, and X. Forty-two (56%) miRNAs had increased expression, while 33 (44%) were downregulated. Of particular interest, four miRNAs that were upregulated in ccRCC, miR-143, miR-146b, miR-340, and miR-378 were mapped to Ch 5q, which is reported to have chromosomal gains in ccRCC. These chromosomal aberrations also agreed with changes reported in the Progenetix database. In addition, the inventors determined that miR-134, miR-487b, and miR-494, which are downregulated in ccRCC, map to Ch 14q, which is reported to be a region of loss of heterozygosity (LOH) in ccRCC. MiR-138, which is downregulated in ccRCC, is located at Ch 3p, which is well-documented to be lost in ccRCC. These data suggest that miRNA dysregulation, at least in part, may be due to chromosomal aberrations in ccRCC.

Hypoxia-inducible miRNAs. The hypoxia-induced pathway has been genetically linked to RCC through the von Hippel-Lindeau (VHL) tumor suppressor gene. Two important pathways, the vascular endothelial growth factor (VEGF) and mammalian target of rapamycin (mTOR), merge in RCC biology at the level of hypoxia-induced factor (HIF), which is mediated upstream, in part, through action of the VHL gene. Under hypoxic conditions, HIF-1α protein accumulates in the cell and plays a central role in renal tumorigenesis by acting as a transcription factor for genes that are involved in angiogenesis, tumor cell proliferation, cell survival and progression, metastatic spread, apoptosis, and glucose metabolism. Recent reports show that miRNAs can also be induced upon hypoxia. The inventors compared their 166 significantly dysregulated miRNAs to miRNAs that have been previously reported to be influenced by hypoxia (61a). They determined that a total of 11 miRNAs previously linked to hypoxia were dysregulated in RCC in the same manner. These are shown in Table 11. Of particular note, the inventors determined that miR-210 is upregulated in RCC. MiR-210 expression has been shown to be progressively increased upon exposure to hypoxic conditions; miR-210 also targets Ephrin-A3 (EFNA3), which is known to be involved in tubulogenesis in the cardiovascular system, suggesting that these proteins may be related to angiogenesis in cancer. These results indicate that miRNAs are downstream effector molecules in the VHL-HIF hypoxia axis, and, therefore, represent new therapeutic targets in RCC and other cancers.

Without being bound by theory, the results obtained in the experiments of Examples 8-12 are discussed below:

The inventors established miRNA expression profiling in clear-cell RCC with qRT-PCR validation. This represents an initial step towards a better understanding of the involvement of miRNAs in RCC pathogenesis and opens the venue for new biomarker discovery and targeted therapy options. The miRNAs identified by microarray analysis and presented in Table 9 show about 54% of them with a significant upregulation (p<0.05), and the remainder being downregulated (p<0.05), a pattern consistent with miRNA expression profiles in other cancers [62a-65a]. Respectively, miR-122, miR-155, and miR-210 showed the highest upregulation levels, whereas miR-200c, miR-335, and miR-218 were the most down-regulated (Table 9).

Out of the highly upregulated miRNAs, miR-210 was identified as being hypoxia-inducible (Table 11) and was also previously documented to be upregulated in other cancers that included breast, lung, prostate and liver (Table 10). Further analysis is needed to investigate the potential involvement of this miRNA in a “common” pathway of cancer development. Similar to the inventors' result, and also based on a miRNA microarray analysis, Camps C. et al, (66a) identified and validated that in a shortage of oxygen levels, miR-210 had the most significant change. They and others showed that this was mediated through the Hypoxia Inducible Factor-1 alpha (HIF-1α) Non Hippel Lindau (VHL) tumor suppressor system (67a-69a).

The tumor suppressor VHL gene is well documented to be inactivated in kidney cancer. It localizes to chromosome 3p25.3 where it acts to prevent tumor growth and is involved in regulating cellular signaling by hypoxia (70a). Hence, its loss will lead to a downstream signaling cascade of events that trigger hypoxia and cause an increase in cellular proliferation leading to cancer progression. HIF-1α is one of the key regulators of hypoxia response and transcription factors, allowing for the regulation of many genes and maintaining steady cell survival under low oxygen levels (71a). Using ChIP (Chromatin Immunoprecipitation) with a HIF-1α antibody, Kulshreshtha et al. (72a) showed that HIF is directly recruited on the promoters of miR-210 and miR-26a under hypoxia, suggesting that hypoxia is involved in miRNA changes in cancer. miR-210 could also link hypoxia and cell cycle regulation in cancer (73a).

Ten out of the 11 hypoxia-inducible miRNAs in Table 11 were found to be upregulated. Extending these results further, one would expect low oxygen levels and signaling due to hypoxic conditions in cancer (74a). This implies that these upregulated miRNAs are induced in low oxygen levels due to tumor growth to inhibit the translation of their target gene, and hence have downstream biological impacts on cell survival and/or proliferation (75a). Based on the inventors' target prediction analysis, some of the hypoxia-inducible miRNAs could be implicated in RCC (FIG. 10). For instance, the VHL gene is targeted by miR-106a and miR-21. Downstream of VHL, miR-199a which the inventors show here as being downregulated in RCC usually targets HIF-1α to suppress the hypoxic effect.

The pro-apoptotic gene BAK1 is a target of miR-26a. miR-26a, as well as miR-210, are documented to have anti-apoptotic effects (76a). In a study by Wong et al. (77a), it was shown that miR-26a over-expression targets the histone methyltransferase, Enhancer of Zeste homolog 2 (Ezh2). Normally, Ezh2 suppresses skeletal muscle differentiation. Thus, this presents an additional role for miR-26a in promoting cellular differentiation.

It has been also reported that miR-21 is commonly upregulated in solid tumors of the lung, breast, stomach, prostate, colon, brain, head and neck, esophagus and pancreas, and here the inventors show kidney. It is directly involved as an oncogene through a mechanism that involves translational repression of the tumor suppressor Programmed Cell Death 4 (PDCD4) gene by promoting cell transformation (78a,79a). Moreover, as an oncogenic miRNA, mir-21 has a role not only in tumor growth but also in invasion and tumor metastasis by targeting and regulating genes like tropomyosin 1 (TPM1), which is another tumor suppressor gene that is involved in cell migration (80a, 81a).

The Akt pathway is implicated in cancer since it causes apoptosis signaling inhibition, and it induces protein synthesis. The tumour suppressor PTEN can lead to the activation of the Akt pathway (82a). It is also implicated in the RCC pathway, and based on the inventors' target mRNA prediction the inventors show that miR-26a can target PTEN and possibly blocks its translation leading to downstream signaling effects that lead to initiation of protein translation, mediated through the mTOR signaling pathway (FIG. 10).

Other miRNAs from Table 9 with known biological roles include miR-20a, miR-29c, miR-126, and miR-424. miR-20a was identified as being upregulated in this study (Table 9) and it also has an anti-apoptotic role as shown in a study by Sylvestre Y et al, (83a) where overexpression of miR-20a decreased apoptosis in a prostate cancer cell line, while its inhibition lead to an increase in cell death. It does so by modulating the translation of the E2F2 and E2F3 mRNAs via binding sites in their 3′-UTR. MiR-29c, another upregulated miRNA has been shown to target genes that encode extracellular matrix proteins, including multiple collagens associated with tumorigenic invasiveness and thus most likely, metastasis (84a). Finally, miR-424 was shown to have an interesting role in controlling the monocyte/macrophage differentiation program (85a).

In a recent study, Gottardo et al. (87a) performed miRNA microarray profiling in kidney and bladder cancers, where they identified four miRNAs—miR-28, miR-185, miR-7-2 and let-7f-2, to be significantly upregulated (and none as downregulated) in kidney cancer with a 1.2 fold change cut-off. None of these miRNAs was identified in the inventors' analysis. Accountability for the differences observed could be attributed to the different experimental techniques and conditions used. For instance, the inventors focused on the clear cell type of kidney cancer (RCC) compared to a mixture of histological types used by Gottardo et al. The inventors also used kidney cortex as a normal control since kidney tumors, specifically of the clear cell type RCC arise from the kidney proximal renal tubular epithelium. The presence of cancer-specific miRNA signatures is not unprecedented. In another interesting study, Sun et al. (88a) showed the presence of five kidney-specific miRNAs—miR-192, miR-194, miR-204, miR-215 and miR-216—which are expressed at much lower levels in other tissues. These miRNAs were found to have high sequence homology that is conserved among species. In addition, highly conserved transcription factor binding sites were found in the upstream sequence of these miRNAs. One of these is a highly conserved binding site for the proto-oncogene ets-1, which is a gene known to be essential for normal development of mammalian kidneys and maintenance of glomerular integrity (89a). Moreover, DNA sequences encoding for some of these miRNAs are located in chromosomal regions associated with kidney cancer (88a). Lastly, the inventors also validated the expression of three of the top dysregulated miRNAs—miR-199a*, miR-122, and miR-200c by qRT-PCR, which is considered a gold standard technique, on three different specimens from those used in the microarray analysis.

In conclusion, the inventors have presented a differential expression profile for miRNAs in kidney cancer. The inventors have also provided evidence that links the biological role of miRNAs to cancer and showed that miRNAs can undertake a variety of mechanisms by which they can affect protein translation by regulating transcription factors and eventually alter processes like apoptosis/cell cycle regulation and cell migration. Further target validation analyses are needed for better understanding of miRNA function.

While the foregoing invention has been described in some detail for purposes of clarity and understanding, it will be appreciated by one skilled in the art, from a reading of the disclosure, that various changes in form and detail can be made without departing from the true scope of the invention in the appended claims. The present invention is not to be limited in scope by the specific embodiments described herein, since such embodiments are intended as but single illustrations of one aspect of the invention, and any functionally equivalent embodiments are within the scope of thereof. Indeed, various modifications of the invention, in addition to those shown and described herein, will become apparent to those skilled in the art from the foregoing description and accompanying drawings. Such modifications are intended to fall within the scope of the appended claims.

All publications, patents, and patent applications referred to herein are incorporated by reference in their entirety to the same extent as if each individual publication, patent, or patent application were specifically and individually indicated to be incorporated by reference in its entirety. Nothing herein is to be construed as an admission that the invention is not entitled to antedate the cited references by virtue of prior invention.

Set out below are full citations for the references cited herein.
1. DeSouza, L., Diehl, G., Rodrigues, M. J., Guo, J., Romaschin, A. D., Colgan, T. J., and Siu, K. W. (2005^{a})_Search for cancer markers from endometrial tissues using differentially labeled tags iTRAQ and cICAT with multidimensional liquid chromatography and tandem mass spectrometry. J Proteome Res. 4, 377-386.
2. McLaughlin, J. K., Lipworth, L., and Tarone, R. E. (2006) Epidemiologic aspects of renal cell carcinoma. Semin Oncol. 33, 527-533.
3. Weiss, R. H. and Lin, P. Y. (2006) Kidney cancer: identification of novel targets for therapy. Kidney Int. 69, 224-232.
4. Ornstein, D. K. and Tyson, D. R. (2006) Proteomics for the identification of new prostate cancer biomarkers. Urol. Oncol. 24, 231-236.
5. Emmert-Buck, M. R., Gillespie, J. W., Paweletz, C. P., Ornstein, D. K., Basrur, V., Appella, E., Wang, Q. H., Huang, J., Hu, N., Taylor, P., and Petricoin, E. F., III (2000) An approach to proteomic analysis of human tumors. Mol Carcinog. 27, 158-165.
6. Shi, T., Dong, F., Liou, L. S., Duan, Z. H., Novick, A. C., and DiDonato, J. A. (2004) Differential protein profiling in renal-cell carcinoma. Mol Carcinog. 40, 47-61.
7. Bradford, T. J., Tomlins, S. A., Wang, X., and Chinnaiyan, A. M. (2006) Molecular markers of prostate cancer. Urol Oncol. 24, 538-551.
8. DeSouza, L., Diehl, G., Yang, E. C., Guo, J., Rodrigues, M. J., Romaschin, A. D., Colgan, T. J., and Siu, K. W. (2005b) Proteomic analysis of the proliferative and secretory phases of the human endometrium: protein identification and differential protein expression. Proteomics. 5, 270-281.
9. DeSouza, L. V., Grigull, J., Ghanny, S., Dube, V., Romaschin, A. D., Colgan, T. J., Siu, K. W. M. (2007) Endometrial carcinoma biomarker discovery and verification using differentially tagged clinical samples with multidimensional liquid chromatography and tandem mass spectrometry. Mol Cell Proteomics. 6, 1170-82.
10. Yang, E. C., Guo, J., Diehl, G., DeSouza, L., Rodrigues, M. J., Romaschin, A. D., Colgan, T. J., and Siu, K. W. (2004) Protein expression profiling of endometrial malignancies reveals a new tumor marker: chaperonin 10. J Proteome Res. 3, 636-643.
11. Grassi, J., Morishita, M., Lewis, P. D., Leonard, R. C., and Thomas, G. (2006) Profiling the breast cancer proteome—the new tool of the future? Clin Oncol. (R. Coll. Radiol.) 18, 581-586.
12. Yu, K. H., Rustgi, A. K., and Blair, I. A. (2005) Characterization of proteins in human pancreatic cancer serum using differential gel electrophoresis and tandem mass spectrometry. J Proteome Res. 4, 1742-1751.
13. Matta, A., DeSouza, L. V., Shukla, N. K., Gupta, S. D., Ralhan, R., and Siu, K. W. (2008) Prognostic Significance of Head-and-Neck Cancer Biomarkers Previously Discovered and Identified Using iTRAQ-Labeling and Multidimensional Liquid Chromatography-Tandem Mass Spectrometry. J Proteome Res. 7, 2078-2087.
14. Ralhan, R., DeSouza, L. V., Matta, A., Chandra, T. S., Ghanny, S., Datta, G. S., Bahadur, S., and Siu, K. W. (2008) Discovery and verification of head-and-neck cancer biomarkers by differential protein expression analysis using iTRAQ-labeling and multidimensional liquid chromatography and tandem mass spectrometry. Mol Cell Proteomics. 7, 1162-73
15. Aebersold, R. and Goodlett, D. R. (2001) Mass spectrometry in proteomics. Chem Rev. 101, 269-295.
16. Gygi, S. P., Rist, B., Gerber, S. A., Turecek, F., Gelb, M. H., and Aebersold, R. (1999) Quantitative analysis of complex protein mixtures using isotope-coded affinity tags. Nat Biotechnol. 17, 994-999.
17. Ross, P. L., Huang, Y. N., Marchese, J. N., Williamson, B., Parker, K., Hattan, S., Khainovski, N., Pillai, S., Dey, S., Daniels, S., Purkayastha, S., Juhasz, P., Martin, S., Bartlet-Jones, M., He, F., Jacobson, A., and Pappin, D. J. (2004) Multiplexed protein quantitation in Saccharomyces cerevisiae using amine-reactive isobaric tagging reagents. Mol Cell Proteomics. 3, 1154-1169.
18. DeSouza, L. V., Taylor, A. M., Li, W., Minkoff, M. S., Romaschin, A. D., Colgan, T. J., Siu, K. W. (2008) Multiple reaction monitoring of mTRAQ labeled peptides enables absolute quantification of endogenous levels of a potential cancer marker in cancerous and normal endometrial tissues. J Proteome Res. 7, 3525-34
19. Ariztia, E. V., Lee, C. J., Gogoi, R., Fishman, D. A. (2006) The tumor microenvironment: key to early detection. Crit Rev Clin Lab Sci. 43, 393-425.
20. Craven, R. A., Stanley, A. J., Hanrahan, S., Dods, J., Unwin, R., Totty, N., Harnden, P., Eardley, I., Selby, P. J., and Banks, R. E. (2006). Proteomic analysis of primary cell lines identifies protein changes present in renal cell carcinoma. Proteomics. 6, 2853-2864.
21. Perego, R. A., Bianchi, C., Corizzato, M., Eroini, B., Torsello, B., Valsecchi, C., Di Fonzo, A., Cordani, N., Favini, P., Ferrero, S., Pitto, M., Sarto, C., Magni, F., Rocco, F., and Mocarelli, P. (2005). Primary cell cultures arising from normal kidney and renal cell carcinoma retain the proteomic profile of corresponding tissues. J Proteome Res. 4, 1503-1510.
22. Sarto, C., Marocchi, A., Sanchez, J. C., Giannone, D., Frutiger, S., Golaz, O., Wilkins, M. R., Doro, G., Cappellano, F., Hughes, G., Hochstrasser, D. F., and Mocarelli, P. (1997.) Renal cell carcinoma and normal kidney protein expression. Electrophoresis. 18, 599-604.
23. Izquierdo, M. A., Schaffer, G. L., Flens, M. J., Giaccone, G., Broxterman, H. J., Meijer, C. J., van, d., V, and Scheper, R. J. (1996) Broad distribution of the multidrug resistance-related vault lung resistance protein in normal human tissues and tumors. Am J Pathol. 148, 877-887.
24. Ferguson, R. E., Jackson, S. M., Stanley, A. J., Joyce, A. D., Harnden, P., Morrison, E. E., Patel, P. M., Phillips, R. M., Selby, P. J., and Banks, R. E. (2005). Intrinsic chemotherapy resistance to the tubulin-binding antimitotic agents in renal cell carcinoma. Int J. Cancer. 115, 155-163.
25. Gagne J. P., Ethier, C., Gagne, P., Mercier, G., Bonicalzi, M. E., Mes-Masson, A. M., Droit, A., Winstall, E., Isabelle, M., and Poirier, G. G. (2007) Comparative proteome analysis of human epithelial ovarian cancer. Proteome Sci. 5, 16.
26. Yao, M., Tabuchi, H., Nagashima, Y., Baba, M., Nakaigawa, N., Ishiguro, H., Hamada, K., Inayama, Y., Kishida, T., Hattori, K., Yamada-Okabe, H., and Kubota, Y. (2005) Gene expression analysis of renal carcinoma: adipose differentiation-related protein as a potential diagnostic and prognostic biomarker for clear-cell renal carcinoma. J Pathol 205, 377-387.
27. Yao, M., Huang, Y., Shioi, K., Hattori, K., Murakami, T., Nakaigawa, N., Kishida, T., Nagashima, Y., and Kubota, Y. (2007). Expression of adipose differentiation-related protein: a predictor of cancer-specific survival in clear cell renal carcinoma. Clin Cancer Res. 13, 152-160.
28. Sartini, D., Santarelli, A., Rossi, V., Goteri, G., Rubini, C., Ciavarella, D., Lo, M. L., and Emanuelli, M. (2007). Nicotinamide N-methyltransferase upregulation inversely correlates with lymph node metastasis in oral squamous cell carcinoma. Mol Med. 13, 415-421.
29. Williams, A. C. and Ramsden, D. B. (2005). Nicotinamide homeostasis: a xenobiotic pathway that is key to development and degenerative diseases. Med Hypotheses 65, 353-362.
30. Kassem, H. S., Sangar, V., Cowan, R., Clarke, N., and Margison, G. P. (2002). A potential role of heat shock proteins and nicotinamide N-methyl transferase in predicting response to radiation in bladder cancer. Int J Cancer. 101, 454-460.
31. Mizutani, Y., Wada, H., Yoshida, O., Fukushima, M., Kawauchi, A., Nakao, M., and Miki, T. (2003). The significance of thymidine phosphorylase/platelet-derived endothelial cell growth factor activity in renal cell carcinoma. Cancer. 98, 730-736.
32. Takayama, T., Mugiya, S., Sugiyama, T., Aoki, T., Furuse, H., Liu, H., Hirano, Y., Kai, F., Ushiyama, T., and Ozono, S. (2006). High levels of thymidine phosphorylase as an independent prognostic factor in renal cell carcinoma. Jpn J Clin Oncol 36, 564-569.
33. Pinder, S. E., Balsitis, M., Ellis, I. O., Landon, M., Mayer, R. J., and Lowe, J. (1994) The expression of alpha B-crystallin in epithelial tumours: a useful tumour marker? J Pathol 174, 209-215.
34. Skopeliti, M., Kratzer, U., Altenberend, F., Panayotou, G., Kalbacher, H., Stevanovic, S., Voelter, W., and Tsitsilonis, O. E. (2007). Proteomic exploitation on prothymosin alpha induced mononuclear cell activation. Proteomics. 7, 1814-1824.
35. Letsas, K. P., Vartholomatos, G., Tsepi, C., Tsatsoulis, A., and Frangou-Lazaridis, M. (2007). Fine-needle aspiration biopsy-RT-PCR expression analysis of prothymosin alpha and parathymosin in thyroid: novel proliferation markers? Neoplasma. 54, 57-62.
36. Mori, M., Barnard, G. F., Staniunas, R. J., Jessup, J. M., Steele, G. D., Jr., and Chen, L. B. (1993) Prothymosin-alpha mRNA expression correlates with that of c-myc in human colon cancer. Oncogene. 8, 2821-2826.
37. Sasaki, H., Nonaka, M., Fujii, Y., Yamakawa, Y., Fukai, I., Kiriyama, M., and Sasaki, M. (2001) Expression of the prothymosin-a gene as a prognostic factor in lung cancer. Surg Today. 31, 936-938.
38. Traub, F., Jost, M., Hess, R., Schorn, K., Menzel, C., Budde, P., Schulz-Knappe, P., Lamping, N., Pich, A., Kreipe, H., and Tammen, H. (2006) Peptidomic analysis of breast cancer reveals a putative surrogate marker for estrogen receptor-negative carcinomas. Lab Invest. 86, 246-253.
39. Tsitsiloni, O. E., Stiakakis, J., Koutselinis, A., Gogas, J., Markopoulos, C., Yialouris, P., Bekris, S., Panoussopoulos, D., Kiortsis, V., Voelter, W., et al., (1993). Expression of alpha thymosins in human tissues in normal and abnormal growth. Proc Natl Acad Sci U.S.A. 90, 9504-9507.
40. Wu, C. G., Habib, N. A., Mitry, R. R., Reitsma, P. H., van Deventer, S. J., and Chamuleau, R. A. (1997). Overexpression of hepatic prothymosin alpha, a novel marker for human hepatocellular carcinoma. Br J Cancer. 76, 1199-1204.
41. Yaffe, M. B., Rittinger, K., Volinia, S., Caron, P. R., Aitken, A., Leffers, H., Gamblin, S. J., Smerdon, S. J., and Cantley, L. C. (1997) The structural basis for 14-3-3:phosphopeptide binding specificity. Cell. 91, 961-971.
42. Ostareck-Lederer, A., Ostareck, D. H., and Hentze, M. W. (1998). Cytoplasmic regulatory functions of the KH-domain proteins hnRNPs K and E1/E2. Trends Biochem Sci. 23, 409-411
43. Roy, M., Xu, Q., and Lee, C. (2005). Evidence that public database records for many cancer associated genes reflect a splice form found in tumors and lack normal splice forms. Nucleic Acids Res. 33, 5026-5033.
1a. Gottardo F, Liu C G, Ferracin M, Calin G A, Fassan M, Bassi P et al.: Micro-RNA profiling in kidney and bladder cancers. Urol Oncol 2007, 25: 387-392.
2a. Garzon R, Pichiorri F, Palumbo T, Visentini M, Aqeilan R, Cimmino A et al.: MicroRNA gene expression during retinoic acid-induced differentiation of human acute promyelocytic leukemia. Oncogene 2007, 26: 4148-4157.
3a. Calin G A, Croce C M: MicroRNA signatures in human cancers. Nat Rev Cancer 2006, 6: 857-866.
4a. Cullen B R: Transcription and processing of human microRNA precursors. Mol Cell 2004, 16: 861-865.
5a. Esquela-Kerscher A, Slack F J: Oncomirs—microRNAs with a role in cancer. Nat Rev Cancer 2006, 6: 259-269.
6a. Fulci V, Chiaretti S, Goldoni M, Azzalin G, Carucci N, Tavolaro S et al.: Quantitative technologies establish a novel microRNA profile of chronic lymphocytic leukemia. Blood 2007, 109: 4944-4951.
7a. Volinia S, Calin G A, Liu C G, Ambs S, Cimmino A, Petrocca F et al.: A microRNA expression signature of human solid tumors defines cancer gene targets. Proc Natl Acad Sci USA 2006, 103: 2257-2261.
8a. Lu J, Getz G, Miska E A, varez-Saavedra E, Lamb J, Peck D et al.: MicroRNA expression profiles classify human cancers. Nature 2005, 435: 834-838.
9a. Nikiforova M N, Tseng G C, Steward D, Diorio D, Nikiforov Y E: MicroRNA expression profiling of thyroid tumors: biological significance and diagnostic utility. J Clin Endocrinol Metab 2008, 93: 1600-1608.
10a. Bandres E, Cubedo E, Agirre X, Malumbres R, Zarate R, Ramirez N et al.: Identification by Real-time PCR of 13 mature microRNAs differentially expressed in colorectal cancer and non-tumoral tissues. Mol Cancer 2006, 5: 29.
11a. Bottoni A, Zatelli M C, Ferracin M, Tagliati F, Piccin D, Vignali C et al.: Identification of differentially expressed microRNAs by microarray: a possible role for microRNA genes in pituitary adenomas. J Cell Physiol 2007, 210: 370-377.
12a. Calin G A, Liu C G, Sevignani C, Ferracin M, Felli N, Dumitru C D et al.: MicroRNA profiling reveals distinct signatures in B cell chronic lymphocytic leukemias. Proc Natl Acad Sci USA 2004, 101: 11755-11760.
13a. Ciafre S A, Galardi S, Mangiola A, Ferracin M, Liu C G, Sabatino G et al.: Extensive modulation of a set of microRNAs in primary glioblastoma. Biochem Biophys Res Commun 2005, 334: 1351-1358.
14a. Fulci V, Chiaretti S, Goldoni M, Azzalin G, Carucci N, Tavolaro S et al.: Quantitative technologies establish a novel microRNA profile of chronic lymphocytic leukemia. Blood 2007, 109: 4944-4951.
15a. Garzon R, Pichiorri F, Palumbo T, Visentini M, Aqeilan R, Cimmino A et al.: MicroRNA gene expression during retinoic acid-induced differentiation of human acute promyelocytic leukemia. Oncogene 2007, 26: 4148-4157.
16a. Gramantieri L, Ferracin M, Formari F, Veronese A, Sabbioni S, Liu C G et al.: Cyclin G1 is a target of miR-122a, a microRNA frequently down-regulated in human hepatocellular carcinoma. Cancer Res 2007, 67: 6092-6099.
17a. Hayashita Y, Osada H, Tatematsu Y, Yamada H, Yanagisawa K, Tomida S et al.: A polycistronic microRNA cluster, miR-17-92, is overexpressed in human lung cancers and enhances cell proliferation. Cancer Res 2005, 65: 9628-9632.
18a. He H, Jazdzewski K, Li W, Liyanarachchi S, Nagy R, Volinia S et al.: The role of microRNA genes in papillary thyroid carcinoma. Proc Natl Acad Sci USA 2005, 102: 19075-19080.
19a. He L, Thomson J M, Hemann M T, Hernando-Monge E, Mu D, Goodson S et al.: A microRNA polycistron as a potential human oncogene. Nature 2005, 435: 828-833.
20a. Iorio M V, Ferracin M, Liu C G, Veronese A, Spizzo R, Sabbioni S et al.: MicroRNA gene expression deregulation in human breast cancer. Cancer Res 2005, 65: 7065-7070.
21a. Lee E J, Gusev Y, Jiang J, Nuovo G J, Lerner M R, Frankel W L et al.: Expression profiling identifies microRNA signature in pancreatic cancer. Int J Cancer 2007, 120: 1046-1054.
22a. Lui W O, Pourmand N, Patterson B K, Fire A: Patterns of known and novel small RNAs in human cervical cancer. Cancer Res 2007, 67: 6031-6043.
23a. Meng F, Henson R, Wehbe-Janek H, Ghoshal K, Jacob S T, Patel T: MicroRNA-21 regulates expression of the PTEN tumor suppressor gene in human hepatocellular cancer. Gastroenterology 2007, 133: 647-658.
24a. Murakami Y, Yasuda T, Saigo K, Urashima T, Toyoda H, Okanoue T et al.: Comprehensive analysis of microRNA expression patterns in hepatocellular carcinoma and non-tumorous tissues. Oncogene 2006, 25: 2537-2545.
25a. Pallante P, Visone R, Ferracin M, Ferraro A, Berlingieri M T, Troncone G et al.: MicroRNA deregulation in human thyroid papillary carcinomas. Endocr Relat Cancer 2006, 13: 497-508.
26a. Porkka K P, Pfeiffer M J, Waltering K K, Vessella R L, Tammela T L, Visakorpi T: MicroRNA expression profiling in prostate cancer. Cancer Res 2007, 67: 6130-6135.
27a. Szafranska A E, Davison T S, John J, Cannon T, Sipos B, Maghnouj A et al.: MicroRNA expression alterations are linked to tumorigenesis and non-neoplastic processes in pancreatic ductal adenocarcinoma. Oncogene 2007, 26: 4442-4452.
28a. Tran N, McLean T, Zhang X, Zhao C J, Thomson J M, O'Brien C et al.: MicroRNA expression profiles in head and neck cancer cell lines. Biochem Biophys Res Commun 2007, 358: 12-17.
29a. Visone R, Pallante P, Vecchione A, Cirombella R, Ferracin M, Ferraro A et al.: Specific microRNAs are downregulated in human thyroid anaplastic carcinomas. Oncogene 2007, 26: 7590-7595.
30a. Volinia S, Calin G A, Liu C G, Ambs S, Cimmino A, Petrocca F et al.: A microRNA expression signature of human solid tumors defines cancer gene targets. Proc Natl Acad Sci USA 2006, 103: 2257-2261.
31a. Wang T, Zhang X, Obijuru L, Laser J, Aris V, Lee P et al.: A micro-RNA signature associated with race, tumor size, and target gene activity in human uterine leiomyomas. Genes Chromosomes Cancer 2007, 46: 336-347.
32a. Yanaihara N, Caplen N, Bowman E, Seike M, Kumamoto K, Yi M et al.: Unique microRNA molecular profiles in lung cancer diagnosis and prognosis. Cancer Cell 2006, 9: 189-198.
33a. Griffiths-Jones S, Saini H K, van D S, Enright A J: miRBase: tools for microRNA genomics. Nucleic Acids Res 2008, 36: D154-D158.
34a. Mitelman F JBaMF. Mitelman Database of Chromosome Aberrations in Cancer (2008). Mitelman F, Johansson B and Mertens F (Eds.). literature. 5-23-0008.
35a. Baudis M. Progenetix molecular cytogenetic online database. literature. 2008.
36a. Bottoni A, Zatelli M C, Ferracin M, Tagliati F, Piccin D, Vignali C et al.: Identification of differentially expressed microRNAs by microarray: a possible role for microRNA genes in pituitary adenomas. J Cell Physiol 2007, 210: 370-377.
37a. Calin G A, Liu C G, Sevignani C, Ferracin M, Felli N, Dumitru C D et al.: MicroRNA profiling reveals distinct signatures in B cell chronic lymphocytic leukemias. Proc Natl Acad Sci USA 2004, 101: 11755-11760.
38a. Ciafre S A, Galardi S, Mangiola A, Ferracin M, Liu C G, Sabatino G et al.: Extensive modulation of a set of microRNAs in primary glioblastoma. Biochem Biophys Res Commun 2005, 334: 1351-1358.
39a. Fulci V, Chiaretti S, Goldoni M, Azzalin G, Carucci N, Tavolaro S et al.: Quantitative technologies establish a novel microRNA profile of chronic lymphocytic leukemia. Blood 2007, 109: 4944-4951.
40a. Garzon R, Pichiorri F, Palumbo T, Visentini M, Aqeilan R, Cimmino A et al.: MicroRNA gene expression during retinoic acid-induced differentiation of human acute promyelocytic leukemia. Oncogene 2007, 26: 4148-4157.
41a. Gramantieri L, Ferracin M, Formari F, Veronese A, Sabbioni S, Liu C G et al.: Cyclin G1 is a target of miR-122a, a microRNA frequently down-regulated in human hepatocellular carcinoma. Cancer Res 2007, 67: 6092-6099.
42a. Hayashita Y, Osada H, Tatematsu Y, Yamada H, Yanagisawa K, Tomida S et al.: A polycistronic microRNA cluster, miR-17-92, is overexpressed in human lung cancers and enhances cell proliferation. Cancer Res 2005, 65: 9628-9632.
43a. He H, Jazdzewski K, Li W, Liyanarachchi S, Nagy R, Volinia S et al.: The role of microRNA genes in papillary thyroid carcinoma. Proc Natl Acad Sci USA 2005, 102: 19075-19080.
44a. He L, Thomson J M, Hemann M T, Hernando-Monge E, Mu D, Goodson S et al.: A microRNA polycistron as a potential human oncogene. Nature 2005, 435: 828-833.
45a. Iorio M V, Ferracin M, Liu C G, Veronese A, Spizzo R, Sabbioni S et al.: MicroRNA gene expression deregulation in human breast cancer. Cancer Res 2005, 65: 7065-7070.
46a. Lee E J, Gusev Y, Jiang J, Nuovo G J, Lerner M R, Frankel W L et al.: Expression profiling identifies microRNA signature in pancreatic cancer. Int J Cancer 2007, 120: 1046-1054.
47a. Lui W O, Pourmand N, Patterson B K, Fire A: Patterns of known and novel small RNAs in human cervical cancer. Cancer Res 2007, 67: 6031-6043.
48a. Meng F, Henson R, Wehbe-Janek H, Ghoshal K, Jacob S T, Patel T: MicroRNA-21 regulates expression of the PTEN tumor suppressor gene in human hepatocellular cancer. Gastroenterology 2007, 133: 647-658.
49a. Murakami Y, Yasuda T, Saigo K, Urashima T, Toyoda H, Okanoue T et al.: Comprehensive analysis of microRNA expression patterns in hepatocellular carcinoma and non-tumorous tissues. Oncogene 2006, 25: 2537-2545.
50a. Pallante P, Visone R, Ferracin M, Ferraro A, Berlingieri M T, Troncone G et al.: MicroRNA deregulation in human thyroid papillary carcinomas. Endocr Relat Cancer 2006, 13: 497-508.
51a. Porkka K P, Pfeiffer M J, Waltering K K, Vessella R L, Tammela T L, Visakorpi T: MicroRNA expression profiling in prostate cancer. Cancer Res 2007, 67: 6130-6135.
52a. Szafranska A E, Davison T S, John J, Cannon T, Sipos B, Maghnouj A et al.: MicroRNA expression alterations are linked to tumorigenesis and non-neoplastic processes in pancreatic ductal adenocarcinoma. Oncogene 2007, 26: 4442-4452.
53a. Tran N, McLean T, Zhang X, Zhao C J, Thomson J M, O'Brien C et al.: MicroRNA expression profiles in head and neck cancer cell lines. Biochem Biophys Res Commun 2007, 358: 12-17.
54a. Visone R, Pallante P, Vecchione A, Cirombella R, Ferracin M, Ferraro A et al.: Specific microRNAs are downregulated in human thyroid anaplastic carcinomas. Oncogene 2007, 26: 7590-7595.
55a. Volinia S, Calin G A, Liu C G, Ambs S, Cimmino A, Petrocca F et al.: A microRNA expression signature of human solid tumors defines cancer gene targets. Proc Natl Acad Sci USA 2006, 103: 2257-2261.
56a. Wang T, Zhang X, Obijuru L, Laser J, Aris V, Lee P et al.: A micro-RNA signature associated with race, tumor size, and target gene activity in human uterine leiomyomas. Genes Chromosomes Cancer 2007, 46: 336-347.
57a. Yanaihara N, Caplen N, Bowman E, Seike M, Kumamoto K, Yi M et al.: Unique microRNA molecular profiles in lung cancer diagnosis and prognosis. Cancer Cell 2006, 9: 189-198.
58a. Blenkiron C, Miska E A: miRNAs in cancer: approaches, aetiology, diagnostics and therapy. Hum Mol Genet 2007, 16 Spec No 1: R106-R113.
59a. Griffiths-Jones S, Saini H K, van D S, Enright A J: miRBase: tools for microRNA genomics. Nucleic Acids Res 2008, 36: D154-D158.
60a. Rathmell W K, Godley P A: Renal cell carcinoma. Curr Opin Oncol 2004, 16: 247-252.
61a. Kulshreshtha R, Ferracin M, Wojcik S E, Garzon R, Alder H, gosto-Perez F J et al.: A microRNA signature of hypoxia. Mol Cell Biol 2007, 27: 1859-1867.
62a. Calin G A, Liu C G, Sevignani C, Ferracin M, Felli N, Dumitru C D et al.: MicroRNA profiling reveals distinct signatures in B cell chronic lymphocytic leukemias. Proc Natl Acad Sci USA 2004, 101: 11755-11760.
63a. Ciafre S A, Galardi S, Mangiola A, Ferracin M, Liu C G, Sabatino G et al.: Extensive modulation of a set of microRNAs in primary glioblastoma. Biochem Biophys Res Commun 2005, 334: 1351-1358.
64a. Garzon R, Pichiorri F, Palumbo T, Visentini M, Aqeilan R, Cimmino A et al.: MicroRNA gene expression during retinoic acid-induced differentiation of human acute promyelocytic leukemia. Oncogene 2007, 26: 4148-4157.
65a. Volinia S, Calin G A, Liu C G, Ambs S, Cimmino A, Petrocca F et al.: A microRNA expression signature of human solid tumors defines cancer gene targets. Proc Natl Acad Sci USA 2006, 103: 2257-2261.
66a. Camps C, Buffa F M, Colella S, Moore J, Sotiriou C, Sheldon H et al.: hsa-miR-210 Is induced by hypoxia and is an independent prognostic factor in breast cancer. Clin Cancer Res 2008, 14: 1340-1348.
67a. Camps C, Buffa F M, Colella S, Moore J, Sotiriou C, Sheldon H et al.: hsa-miR-210 Is induced by hypoxia and is an independent prognostic factor in breast cancer. Clin Cancer Res 2008, 14: 1340-1348.
68a. Fasanaro P, D'Alessandra Y, Di S, V, Melchionna R, Romani S, Pompilio G et al.: MicroRNA-210 Modulates Endothelial Cell Response to Hypoxia and Inhibits the Receptor Tyrosine Kinase Ligand Ephrin-A3. J Biol Chem 2008, 283: 15878-15883.
69a. Pulkkinen K, Malm T, Turunen M, Koistinaho J, Yla-Herttuala S: Hypoxia induces microRNA miR-210 in vitro and in vivo Ephrin-A3 and neuronal pentraxin 1 are potentially regulated by miR-210. FEBS Lett 2008.
70a. Li L, Zhang L, Zhang X, Yan Q, Minamishima Y A, Olumi A F et al.: Hypoxia-inducible factor linked to differential kidney cancer risk seen with type 2A and type 2B VHL mutations. Mol Cell Biol 2007, 27: 5381-5392.
71a. Kulshreshtha R, Ferracin M, Wojcik S E, Garzon R, Alder H, gosto-Perez F J et al.: A microRNA signature of hypoxia. Mol Cell Biol 2007, 27: 1859-1867.
72a. Kulshreshtha R, Ferracin M, Wojcik S E, Garzon R, Alder H, gosto-Perez F J et al.: A microRNA signature of hypoxia. Mol Cell Biol 2007, 27: 1859-1867.
73a. Giannakakis A, Sandaltzopoulos R, Greshock J, Liang S, Huang J, Hasegawa K et al.: miR-210 links hypoxia with cell cycle regulation and is deleted in human epithelial ovarian cancer. Cancer Biol Ther 2007, 7.
74a. Giannakakis A, Sandaltzopoulos R, Greshock J, Liang S, Huang J, Hasegawa K et al.: miR-210 links hypoxia with cell cycle regulation and is deleted in human epithelial ovarian cancer. Cancer Biol Ther 2007, 7.
75a. Cho W C: OncomiR5: the discovery and progress of microRNAs in cancers. Mol Cancer 2007, 6: 60.
76a. Kulshreshtha R, Ferracin M, Wojcik S E, Garzon R, Alder H, gosto-Perez F J et al.: A microRNA signature of hypoxia. Mol Cell Biol 2007, 27: 1859-1867.
77a. Wong C F, Tellam R L: MicroRNA-26a targets the histone methyltransferase Enhancer of Zeste homolog 2 during myogenesis. J Biol Chem 2008, 283: 9836-9843.
78a. Verghese E, Hanby A, Speirs V, Hughes T: Small is beautiful: microRNAs and breast cancer—where are the inventors now? J Pathol 2008, 215: 214-221.
79a. Lu Z, Liu M, Stribinskis V, Klinge C M, Ramos K S, Colburn N H et al.: MicroRNA-21 promotes cell transformation by targeting the programmed cell death 4 gene. Oncogene 2008.
80a. Zhu S, Si M L, Wu H, Mo Y Y: MicroRNA-21 targets the tumor suppressor gene tropomyosin 1 (TPM1). J Biol Chem 2007, 282: 14328-14336.
81a. Zhu S, Wu H, Wu F, Nie D, Sheng S, Mo Y Y: MicroRNA-21 targets tumor suppressor genes in invasion and metastasis. Cell Res 2008, 18: 350-359.
82a. Yang H, Kong W, He L, Zhao J J, O'Donnell J D, Wang J et al.: MicroRNA expression profiling in human ovarian cancer: miR-214 induces cell survival and cisplatin resistance by targeting PTEN. Cancer Res 2008, 68: 425-433.
83a. Sylvestre Y, De G, V, Querido E, Mukhopadhyay U K, Bourdeau V, Major F et al.: An E2F/miR-20a autoregulatory feedback loop. J Biol Chem 2007, 282: 2135-2143.
84a. Sengupta S, den Boon J A, Chen I H, Newton M A, Stanhope S A, Cheng Y J et al.: MicroRNA 29c is down-regulated in nasopharyngeal carcinomas, up-regulating mRNAs encoding extracellular matrix proteins. Proc Natl Acad Sci USA 2008, 105: 5874-5878.
85a. Rosa A, Ballarino M, Sorrentino A, Sthandier O, De Angelis F G, Marchioni M et al.: The interplay between the master transcription factor PU.1 and miR-424 regulates human monocyte/macrophage differentiation. Proc Natl Acad Sci USA 2007, 104: 19849-19854.
86a. Wenguang Z, Jianghong W, Jinquan L, Yashizawa M: A subset of skin-expressed microRNAs with possible roles in goat and sheep hair growth based on expression profiling of mammalian microRNAs. OMICS 2007, 11: 385-396.
87a. Gottardo F, Liu C G, Ferracin M, Calin G A, Fassan M, Bassi P et al.: Micro-RNA profiling in kidney and bladder cancers. Urol Oncol 2007, 25: 387-392.
88a. Sun Y, Koo S, White N, Peralta E, Esau C, Dean N M et al.: Development of a micro-array to detect human and mouse microRNAs and characterization of expression in human organs. Nucleic Acids Res 2004, 32: e188.
89a. Razzaque M S, Naito T, Taguchi T: Proto-oncogene Ets-1 and the kidney. Nephron 2001, 89: 1-4.

TABLE 1
Distribution of proteins and their expression levels
in RCC compared to their normal counterparts.

| | | Expression levels | | |
|---|---|---|---|---|
| | | No of Proteins | | |
| | | <0.67 | 0.67-1.50 | >1.50 |
| First run | 574 | 107 | 351 | 116 |
| Second run | 626 | 110 | 424 | 89 |
| Proteins identified in | 262 | 65* | 163* | 34* |
| both runs | | | | |
| Proteins identified in | 937 | 168* | 613* | 156* |
| either of the runs | | | | |

*Average of the expression levels in both runs.

TABLE 2
A partial list of differentially expressed proteins in kidney cancer tissue
compared to normal counterparts from the same patient.

| | | Gene | SwissProt | Fold | |
|---|---|---|---|---|---|
| No. | Protein Name | Symbol | ID | Change1 | Regulation1 |
| 1 | KIAA1865 protein | C14orf4 | Q9H1B7 | 4.8624 | UP |
| 2 | tyrosine 3/tryptophan 5-monooxygenase | YWHAH | Q04917 | 4.2912 | UP |
| | activation protein | | | | |
| 3 | CAPNS1 protein | CAPNS1 | P04632 | 3.9777 | UP |
| 4 | Complement component 1 inhibitor | SERPING1 | Q96FE0 | 3.66505 | UP |
| 5 | UDP-glucose 6-dehydrogenase | UGDH | O60701 | 3.3497 | UP |
| 6 | L-lactate dehydrogenase A chain | LDHA | P00338 | 3.32095 | UP |
| 7 | Nicotinamide N-methyltransferase | NNMT | P40261 | 3.2572 | UP |
| 8 | Hypothetical protein DKFZp686H13163 | GSTO1 | Q7Z3T2 | 3.1252 | UP |
| 9 | Poly(RC)-binding protein 2, isoform b | PCBP2 | Q6PKG5 | 3.0866 | UP |
| 10 | ADP-ribosylation factor 3 | ARF3 | P61204 | 3.0753 | UP |
| 11 | Calnexin precursor | CANX | P27824 | 2.8414 | UP |
| 12 | 3′(2′),5′-bisphosphate nucleotidase 1 | BPNT1 | O95861 | 2.7755 | UP |
| 13 | Hypothetical protein DKFZp547J2313 | FABP7 | Q9H047 | 2.7722 | UP |
| 14 | Catechol O-methyltransferase, membrane- | COMT | P21964 | 2.7512 | UP |
| | bound form | | | | |
| 15 | Glyceraldehyde-3-phosphate dehydrogenase, | GAPDHS | O14556 | 2.6716 | UP |
| | testis-specific | | | | |
| 16 | Hypothetical protein | ANXA4 | Q6P452 | 2.6156 | UP |
| 17 | Hypothetical protein DKFZp686I04222 | SERPINB6 | Q7Z2Y7 | 2.5934 | UP |
| 18 | Echinoderm microtubule-associated protein-like 4 | EML4 | Q9HC35 | 2.575 | UP |
| 19 | vimentin - human | VIM | P08670 | 2.5615 | UP |
| 20 | Cytoplasmic dynein intermediate chain 2C | DYNC1I2 | Q7Z4X1 | 2.5234 | UP |
| 21 | Calpain small subunit 1 | CAPNS1 | P04632 | 2.5198 | UP |
| 22 | Glutathione S-transferase | GSTA2 | P09210 | 2.49785 | UP |
| 23 | Alpha crystallin β | CRYAB | P02511 | 2.48945 | UP |
| 24 | ALDOC protein | ALDOC | Q6P0L5 | 2.4321 | UP |
| 25 | Rab GDP dissociation inhibitor alpha | GDI1 | P31150 | 2.4198 | UP |
| 26 | PRKAR2A protein | PRKAR2A | Q9BUB1 | 2.4146 | UP |
| 27 | Chloride intracellular channel protein 1 | CLIC1 | O00299 | 2.4112 | UP |
| 28 | Pre-B-cell colony enhancing factor 1, isoform b | PBEF1 | Q8WW95 | 2.4046 | UP |
| 29 | Annexin A5 | ANXA5 | P08758 | 2.3679 | UP |
| 30 | glyceraldehyde-3-phosphate dehydrogenase | GAPDH | P04406 | 2.3475 | UP |
| 31 | Endothelial cell growth factor 1 (platelet-derived) | ECGF1 | P19971 | 1.834 | UP |
| 32 | Major vault protein | MVP | Q14764 | 1.6983 | UP |
| 33 | Adipose differentiation-related protein | ADFP | Q99541 | 1.6629 | UP |
| 34 | 60 kDa heat shock protein | HSPD1 | P10809 | 0.4896 | DOWN |
| 35 | ATP synthase delta chain | ATP5D | P30049 | 0.4879 | DOWN |
| 36 | Coronin 1A | CORO1A | P31146 | 0.4878 | DOWN |
| 37 | GCSH protein | GCSH | Q6IAT2 | 0.4873 | DOWN |
| 38 | TAGLN protein | TAGLN | Q6FI52 | 0.4862 | DOWN |
| 39 | Ksp-cadherin | CDH16 | Q6UW93 | 0.4818 | DOWN |
| 40 | splicing factor, arginine/serine-rich 2 | SFRS2IP | Q99590 | 0.4802 | DOWN |
| 41 | Zinc finger protein 207 | ZNF207 | O43670 | 0.4726 | DOWN |
| 42 | 40S ribosomal protein S17 | RPS17 | P08708 | 0.4702 | DOWN |
| 43 | Secreted cement gland protein XAG-2 homolog | AGR2 | O95994 | 0.46395 | DOWN |
| 44 | Thymosin beta-4 | TMSB4X | P62328 | 0.4631 | DOWN |
| 45 | CKB protein | CKB | Q6FG40 | 0.4589 | DOWN |
| 46 | Calmodulin | CALM1 | Q13942 | 0.4562 | DOWN |
| 47 | Hypothetical protein FLJ46684 | C9orf58 | Q6ZR40 | 0.4479 | DOWN |
| 48 | Elongation factor Tu | TUFM | P49411 | 0.4476 | DOWN |
| 49 | Tumor protein p53 inducible protein 3 | TP53I3 | Q9BWB8 | 0.4383 | DOWN |
| 50 | Calmodulin | CALM1 | P62158 | 0.4245 | DOWN |
| 51 | Hypothetical protein DKFZp586K2222 | TPM1 | Q9Y427 | 0.4187 | DOWN |
| 52 | creatine kinase-B | CKB | P12277 | 0.4162 | DOWN |
| 53 | ATP synthase beta chain | ATP5B | P06576 | 0.41455 | DOWN |
| 54 | Hemoglobin beta | HBB | Q6R7N2 | 0.4127 | DOWN |
| 55 | Histone H2A | HIST3H2A | Q7L7L0 | 0.4112 | DOWN |
| 56 | AP endonuclease 1 | APEX1 | P27695 | 0.4085 | DOWN |
| 57 | Acyl-CoA dehydrogenase, medium-chain specific | ACADM | P11310 | 0.3938 | DOWN |
| 58 | Nonhistone chromosomal protein HMG-17 | HMGN2 | P05204 | 0.39295 | DOWN |
| 59 | HES1 protein | C21orf33 | P30042 | 0.3925 | DOWN |
| 60 | Eukaryotic translation initiation factor 3 subunit 3 | EIF3H | O15372 | 0.3894 | DOWN |
| 61 | Calreticulin | CALB2 | Q96BK4 | 0.3772 | DOWN |
| 62 | Programmed cell death protein 5 | PDCD5 | O14737 | 0.3623 | DOWN |
| 63 | Chromosome 10 open reading frame 65 | C10orf65 | Q86XE5 | 0.3616 | DOWN |
| 64 | adenylate kinase 3 alpha | AK3 | Q9UIJ7 | 0.3613 | DOWN |
| 65 | LOC112817 protein | C10orf65 | Q96EV5 | 0.3554 | DOWN |
| 66 | Ubiquinol-cytochrome-c reductase complex core | UQCRC1 | P31930 | 0.3531 | DOWN |
| | protein I | | | | |
| 67 | Plastin 3 | PLS3 | Q86YI6 | 0.3394 | DOWN |
| 68 | Membrane associated protein SLP-2 | STOML2 | Q9UJZ1 | 0.3341 | DOWN |
| 69 | MHC class II antigen | HLA- | Q9MYD9 | 0.3294 | DOWN |
| | | DRB1 | | | |
| 70 | Reticulocalbin 1 precursor | RCN1 | Q15293 | 0.312 | DOWN |
| 71 | DNA-binding protein B | YBX1 | P67809 | 0.31 | DOWN |
| 72 | Cytochrome c | CYCS | Q6NUR2 | 0.2901 | DOWN |
| 73 | NADH-ubiquinone oxidoreductase 13 kDa-A | NDUFS6 | O75380 | 0.2759 | DOWN |
| | subunit | | | | |
| 74 | Lupus La protein | SSB | P05455 | 0.2709 | DOWN |
| 75 | Pyruvate dehydrogenase E1 component beta | PDHB | P11177 | 0.2662 | DOWN |
| | subunit | | | | |
| 76 | FERM, RhoGEF, and pleckstrin domain protein 1, | FARP1 | Q9Y4F1 | 0.2628 | DOWN |
| | isoform 1 | | | | |
| 77 | Mitochondrial aldehyde dehydrogenase 2 | ALDH2 | Q6IV71 | 0.2603 | DOWN |
| 78 | 2,4-dienoyl-CoA reductase | DECR1 | Q16698 | 0.21255 | DOWN |
| 79 | OXCT protein | OXCT1 | Q6IAV5 | 0.1973 | DOWN |
| 80 | Mitochondrial glycine cleavage system H-protein | GCSH | Q6QN92 | 0.164 | DOWN |
| 81 | Prothymosin alpha | PTMA | Q15202 | 0.1322 | DOWN |
| 82 | Hypothetical protein DKFZp564K0164 | PDHB | Q9UFK3 | 0.1253 | DOWN |
| 83 | Hemoglobin beta | HBB | Q6J1Z7 | 0.0835 | DOWN |
| 84 | Hypothetical protein DKFZp761J19 | C9orf58 | Q9BQI0 | 0.0412 | DOWN |

TABLE 3
Protein expression¹ in RCC sample compared to
non-cancerous renal failure and transitional cell carcinoma (TCC).

| | | No of proteins (%) with expression | | |
|---|---|---|---|---|
| | | status: | | |
| | Mean ± SE | <0.67 | 0.67-1.50 | >1.50 |
| RCC: renal failure | 1.088 ± 0.016 | 151 (16.1) | 637 (68.0) | 149 (15.9) |
| RCC: TCC | 1.156 ± 0.018 | 148 (15.8) | 614 (65.5) | 175 (18.7) |

¹Protein expression levels were normalized by normal kidney tissue expression and expressed as fold changes from normal.

TABLE 4
Dysregulated proteins that were found to be differentially expressed
in kidney cancer by other independent reports.

| | Swiss Prot | Craven | Perego | Sarto | |
|---|---|---|---|---|---|
| Protein | ID | 22 | 23 | 18 | Shi 5 |
| 60 kDa heat shock protein | P10809 | | | | |
| 60S acidic ribosomal | P05387 | | | | |
| protein P2 | | | | | |
| 78 kDa glucose-regulated | P11021 | | | | |
| protein | | | | | |
| Albumin | P02768 | | | | |
| Alpha Crystallin B chain | P02511 | | | | |
| Alpha enolase | P06733 | | | | |
| Aminoacylase | Q03154 | | | | |
| ATP synthase D chain | P30049 | | | | |
| Calreticulin | P27797 | | | | |
| Cathepsin B | P07858 | | | | |
| Cathepsin D | P07339 | | | | |
| Elongation factor 1-beta | P24534 | | | | |
| Elongation factor 2 | P13639 | | | | |
| Glutamate dehydrogenase | P00367 | | | | |
| Glutathione S transferase | P09211 | | | | |
| Heat shock 27 kDa protein | P04792 | | | | |
| Heat shock cognate 71 kDa | P11142 | | | | |
| protein | | | | | |
| Heterogeneous nuclear | P22626 | | | | |
| ribonucleoprotein A2/B1 | | | | | |
| Lactate dehydrogenase A | P00338 | | | | |
| Lactate dehydrogenase B | P07195 | | | | |
| Peptidyl-prolyl cis-trans | P23284 | | | | |
| isomerase B | | | | | |
| Protein disulfide isomerase | P07237 | | | | |
| Septin 2 | Q15019 | | | | |
| Tropomyosin alpha 3 chain | P06753 | | | | |
| Total Proteins identified: | 243 | 34 | 28 | 27 | 23 |

TABLE 5
Partial list of proteins with documented kidney expression
in the SwissProt database.

| | Gene |
|---|---|
| SwissProt | symbol |
| Q03154 | ACY1 |
| P30038 | ALDH4A1 |
| P49189 | ALDH9A1 |
| P05090 | APOD |
| Q93088 | BHMT |
| P46940 | IQGAP1 |
| O00151 | PDLIM1 |
| Q01082 | SPTBN1 |
| P27348 | YWHAQ |
| Q9UKL9 | AKR1C3 |
| Q6DN90 | IQSEC1 |
| Q8IZK5 | LDHD |
| Q6IPK9 | CES2 |
| Q6I9V1 | DPEP1 |
| O75531 | BANF1 |
| Q16698 | DECR1 |
| Q96H54 | ECHS1 |
| Q96CU2 | EEF1G |
| Q9Y4F1 | FARP1 |
| P35558 | PCK1 |
| Q9Y371 | SH3GLB1 |
| O95994 | AGR2 |
| P38117 | ETFB |

TABLE 6
Comparison between protein and SAGE mRNA expression data
for upregulated proteins that were detected in both runs.

| | | |
|---|---|---|
| | | SAGE² Normal |
| Gene Symbol | Protein¹ | Cancer |

| | | | |
|---|---|---|---|
| SERPING1 | 3.6651 | 9 | 31 |
| LDHA | 3.3210 | 29 | 71 |
| FABP7 | 2.7722 | 1 | 6 |
| GAPDHS | 2.6716 | 1 | 3 |
| CRYAB | 2.4895 | 136 | 5 |
| CLIC1 | 2.4112 | 48 | 57 |
| ANXA5 | 2.3679 | 4 | 25 |
| GAPDH | 2.2959 | 87 | 1326 |
| HPX | 2.2292 | 1 | 4 |
| PCBP1 | 2.1984 | 24 | 15 |
| KRT18 | 2.1134 | 39 | 239 |
| ENO1 | 2.0265 | 1 | 15 |
| PRDX6 | 1.9705 | 4 | 23 |
| ACTB | 1.8686 | 331 | 285 |
| CNDP2 | 1.8460 | 53 | 167 |
| AKR1A1 | 1.8214 | 19 | 25 |
| PTBP1 | 1.8200 | 1 | 15 |
| NDRG1 | 1.8022 | 4 | 9 |

¹Protein expression is expressed as the fold change of expression of cancer: normal.
²SAGE expression data are presented as relative expression value (tag/200,000 tags).

TABLE 7
A partial list of consistently overexpressed proteins (in both runs)
in RCC compared to two different in silico EST databases.

| | | | Digital Gene | | |
|---|---|---|---|---|---|
| | | | Expression | | |
| Gene | EST ProfileViewer¹ | | Displayer² | | |
| symbol | Normal Cancer | | Normal Cancer | | Overall value |
| ACTB | 3090 | 3631 | 111 | 185 | Increased in both |
| ACTR3 | 291 | 86 | 0 | 1 | Increased in one |
| AKR1A1 | 136 | 144 | N/A | N/A | Increased in one |
| ALB | 1180 | 14 | 155 | 0 | Decreased in |
| | | | | | both |
| ANXA5 | 112 | 187 | 7 | 11 | Increased in both |
| CLIC1 | 211 | 446 | 2 | 34 | Increased in both |
| CNDP2 | 508 | 489 | 57 | 26 | Decreased in |
| | | | | | both |
| CTSD | 329 | 879 | 2 | 0 | Increased in one |
| DCTN2 | 230 | 230 | 7 | 15 | Increased in one |
| DYNC1I2 | 235 | 230 | 28 | 3 | Decreased in |
| | | | | | both |
| EEF2 | 197 | 288 | 4 | 15 | Increased in both |
| FABP7 | 32 | 14 | 0 | 7 | Increased in one |
| FAM49B | 51 | 57 | 4 | 3 | Increased in one |
| GAPDH | 2309 | 6066 | 11 | 260 | Increased in both |
| GAPDHS | 9 | 14 | 1 | 0 | Increased in one |
| GSTA2 | 42 | 14 | 5 | 3 | Decreased in |
| | | | | | both |
| KRT18 | 310 | 547 | 6 | 38 | Increased in both |
| LDHA | 870 | 835 | 47 | 52 | Increased in one |
| MTPN | 23 | 28 | 4 | 7 | Increased in both |
| MVP | 399 | 244 | 65 | 7 | Decreased in |
| | | | | | both |
| PCBP1 | 89 | 115 | 0 | 7 | Increased in both |
| PRDX6 | 225 | 259 | 22 | 15 | Increased in one |
| PTBP1 | 169 | 288 | 14 | 16 | Increased in both |

¹EST ProfileViewer values are calculated as transcripts/million
²Digital gene expression displayer values are calculated as sequence odds ratio.

TABLE 8
List of novel differentially expressed proteins in RCC tissue compared to normal
counterparts from the same patient.

| SEQ | | | | SwissProt | Average Fold |
|---|---|---|---|---|---|
| NO. | Protein Name | Gene Symbol | Accession # | ID | Change |
| 1 | tyrosine 3/tryptophan 5- | YWHAH | rf\|NP_003396.1 | Q04917 | Upregulated |
| | monooxygenase activation | | | | |
| | protein, eta polypeptide | | | | |
| 2 | Calpain, small subunit 1 | CAPNS1 | gb\|AAH64998.1 | P04632 | Upregulated |
| 4 | Serpin peptidase inhibitor, clade G | SERPING1 | trm\|Q96FE0 | Q96FE0 | Upregulated |
| | (C1 inhibitor), member 1, | | | | |
| | (angioedema, hereditary) | | | | |
| 5 | Poly(rC) binding protein 2 | PCBP2 | trm\|Q6PKG5 | Q6PKG5 | Upregulated |
| 6 | Chromosome 14 open reading | C14orf4 | dbj\|BAB47494.1 | Q9H1B7 | Upregulated |
| | frame 4 | | | | |
| 7 | UDP-glucose dehydrogenase | UGDH | spt\|O60701 | O60701 | Upregulated |
| 8 | Lactate dehydrogenase A | LDHA | spt\|P00338 | P00338 | Upregulated |
| 9 | Glutathione S-transferase omega 1 | GSTO1 | trm\|Q7Z3T2 | Q7Z3T2 | Upregulated |
| 10 | annexin A2 isoform 1 | ANXA2 | rf\|NP_001002858.1 | P07355 | Upregulated |
| 11 | Calnexin | CANX | spt\|P27824 | P27824 | Upregulated |
| 12 | 3′(2′),5′-bisphosphate | BPNT1 | spt\|O95861 | O95861 | Upregulated |
| | nucleotidase 1 | | | | |
| 13 | Echinoderm microtubule | EML4 | spt\|Q9HC35 | Q9HC35 | Upregulated |
| | associated protein like 4 | | | | |
| 14 | Protein kinase, cAMP-dependent, | PRKAR2A | trm\|Q9BUB1 | Q9BUB1 | Upregulated |
| | regulatory, type II, alpha | | | | |
| 15 | Chloride intracellular channel 1 | CLIC1 | spt\|O00299 | O00299 | Upregulated |
| 16 | In multiple clusters | ANXA5 | spt\|P08758 | P08758 | Upregulated |
| 17 | Kininogen 1 | KNG1 | spt\|P01042 | P01042 | Upregulated |
| 18 | Dipeptidase 1 (renal) | DPEP1 | trm\|Q6I9V1 | Q6I9V1 | Upregulated |
| 19 | Annexin A1 | ANXA1 | spt\|P04083 | P04083 | Upregulated |
| 20 | RAB5C, member RAS oncogene | RAB5C | pir\|I38703 | P51148 | Upregulated |
| | family | | | | |
| 21 | Cell division cycle 37 homolog (S. cerevisiae) | CDC37 | spt\|Q16543 | Q16543 | Upregulated |
| 22 | Prothymosin alpha | PTMA | trm\|Q15202 | Q15202 | Downregulated |
| 23 | Chromosome 9 open reading | C9orf58 | trm\|Q9BQI0 | Q9BQI0 | Downregulated |
| | frame 58 | | | | |
| 24 | 3-oxoacid CoA transferase 1 | OXCT1 | trm\|Q6IAV5 | Q6IAV5 | Downregulated |
| 25 | Aldehyde dehydrogenase 2 family | ALDH2 | trm\|Q6IV71 | Q6IV71 | Downregulated |
| | (mitochondrial) | | | | |
| 26 | FERM, RhoGEF (ARHGEF) and | FARP1 | trm\|Q9Y4F1 | Q9Y4F1 | Downregulated |
| | pleckstrin domain protein 1 | | | | |
| | (chondrocyte-derived) | | | | |
| 27 | Dihydroxyacetone kinase 2 | DAK | trm\|Q9H895 | Q9H895 | Downregulated |
| | homolog (S. cerevisiae) | | | | |
| 28 | Y box binding protein 1 | YBX1 | gb\|AAA35750.1 | P67809 | Downregulated |
| 29 | Nuclear protein Hcc-1 (HSPC316) | CIP29 | cra\|hCP1880337 | P82979 | Downregulated |
| | (Proliferation associated cytokine- | | | | |
| | inducible protein CIP29) | | | | |
| 30 | Adenylate kinase 3 | AK3 | dbj\|BAA87913.1 | Q9UIJ7 | Downregulated |
| 31 | Programmed cell death 5 | PDCD5 | spt\|O14737 | O14737 | Downregulated |
| 32 | 3-hydroxyisobutyryl-CoA | HIBCH | cra\|hCP1793588 | Q6NVY1 | Downregulated |
| | hydrolase, mitochondrial | | | | |
| | [Precursor] | | | | |
| 33 | Eukaryotic translation initiation | EIF3H | spt\|O15372 | O15372 | Downregulated |
| | factor 3, subunit H | | | | |
| 34 | High-mobility group nucleosomal | HMGN2 | spt\|P05204 | P05204 | Downregulated |
| | binding domain 2 | | | | |
| 35 | APEX nuclease (multifunctional | APEX1 | spt\|P27695 | P27695 | Downregulated |
| | DNA repair enzyme) 1 | | | | |
| 36 | In multiple clusters | TMSB4X | spt\|P62328 | P62328 | Downregulated |
| 37 | Anterior gradient homolog 2 | AGR2 | trm\|O95994 | O95994 | Downregulated |
| | (Xenopus laevis) | | | | |
| 38 | Barrier to autointegration factor 1 | BANF1 | spt\|O75531 | O75531 | Downregulated |
| 39 | Coronin, actin binding protein, 1A | CORO1A | spt\|P31146 | P31146 | Downregulated |
| 40 | Lectin, galactoside-binding, | LGALS3BP | spt\|Q08380 | Q08380 | Downregulated |
| | soluble, 3 binding protein | | | | |

TABLE 9
The top 35 miRNAs differentially expressed in clear-cell
RCC compared to normal counterparts.

| | p- | Fold | Chro. | | |
|---|---|---|---|---|---|
| miRNA | value | Change | Arm | Band | Sequence |
| Upregulated | | | | | |
| hsa-miR-122 | 28.16 | <1e−07 | 18q | 21 | UGGAGUGUGACAAUGGUGUUUG |
| hsa-miR-155 | 11.24 | 1.00E−07 | 21q | 21 | UUAAUGCUAAUCGUGAUAGGGU |
| hsa-miR-210 | 9.47 | <1e−07 | 11p | 15 | CUGUGCGUGUGACAGCGGCUGA |
| hsa-miR-34a | 4.90 | 3.00E−07 | 1p | 36 | UGGCAGUGUCUUAGCUGGUUGU |
| hsa-miR-15a | 3.76 | 5.80E−06 | 13q | 14 | UAGCAGCACAUAAUGGUUUGUG |
| hsa-miR-223 | 3.53 | 0.00017 | Xq | 12 | UGUCAGUUUGUCAAAUACCCCA |
| hsa-miR-21 | 3.16 | 5.70E−07 | 17q | 23 | UAGCUUAUCAGACUGAUGUUGA |
| hsa-miR-451 | 3.06 | 0.004842 | 17q | 11 | AAACCGUUACCAUUACUGAGUU |
| hsa-miR-15b | 3.04 | 6.00E−07 | 3q | 26 | UAGCAGCACAUCAUGGUUUACA |
| hsa-miR-342-3p | 3.03 | 2.45E−05 | 14q | 32 | UCUCACACAGAAAUCGCACCCGU |
| hsa-miR-224 | 3.01 | 8.60E−06 | Xq | 28 | CAAGUCACUAGUGGUUCCGUU |
| hsa-miR-486-5p | 2.89 | 0.006009 | 8p | 11 | UCCUGUACUGAGCUGCCCCGAG |
| hsa-miR-106b | 2.78 | 6.00E−07 | 7q | 22 | UAAAGUGCUGACAGUGCAGAU |
| hsa-miR-150 | 2.62 | 0.013689 | 19q | 13 | UCUCCCAACCCUUGUACCAGUG |
| hsa-miR-342-5p | 2.32 | 1.00E−06 | 14q | 32 | AGGGGUGCUAUCUGUGAUUGA |
| hsa-miR-146b-5p | 2.27 | 0.001901 | 10q | 24 | UGAGAACUGAAUUCCAUAGGCU |
| hsa-miR-130b | 2.22 | <1e−07 | 22q | 11 | CAGUGCAAUGAUGAAAGGGCAU |
| hsa-miR-1270 | 2.15 | 7.76E−05 | 19p | 12 | CUGGAGAUAUGGAAGAGCUGUGU |
| Downregulated | | | | | |
| hsa-miR-200c | 0.05 | <1e−07 | 12p | 13 | UAAUACGCGGGUAAUGAUGGA |
| hsa-miR-335 | 0.16 | 5.00E−07 | 7q | 32 | UCAAGAGCAAUAACGAAAAAUGU |
| hsa-miR-218 | 0.18 | 1.40E−06 | 4p | 15 | UUGUGCUUGAUCUAACCAUGU |
| hsa-miR-1974 | 0.18 | 3.00E−07 | 5q | 15 | UGGUUGUAGUCCGUGCGAGAAUA |
| hsa-miR-1246 | 0.22 | 0.000485 | 2q | 31 | AAUGGAUUUUUGGAGCAGG |
| hsa-miR-1977 | 0.23 | 2.00E−07 | 1p | 36 | GAUTAUUUTGCUUAGCUGUUAA |
| hsa-miR-200b | 0.24 | 2.20E−06 | 1p | 36 | UAAUACUGCCUGGUAAUGAUGA |
| hsa-miR-429 | 0.26 | 2.00E−07 | 1p | 36 | UAAUACUGUCUGGUAAAACCGU |
| hsa-miR-187 | 0.28 | 5.70E−06 | 18q | 12 | UCGUGUCUUGUGUUGCAGCCGG |
| hsa-miR-532-5p | 0.29 | <1e−07 | Xp | 11 | CAUGCCUUGAGUGUAGGACCGU |
| hsa-miR-204 | 0.30 | 0.001959 | 9q | 21 | UUCCCUUUGUCAUCCUAUGCCU |
| hsa-miR-30e | 0.33 | 2.12E−05 | 1p | 34 | UGUAAACAUCCUUGACUGGAAG |
| hsa-miR-363 | 0.34 | 8.00E−07 | Xq | 26 | AAUUGCACGGUAUCCAUCUGUA |
| hsa-miR-10a | 0.34 | 4.98E−05 | 17q | 21 | UACCCUGUAGAUCCGAAUUUGUG |
| hsa-miR-532-3p | 0.35 | 4.99E−05 | Xp | 11 | CCUCCCACACCCAAGGCUUGCA |
| hsa-miR-660 | 0.36 | 2.00E−07 | Xp | 11 | UACCCAUUGCAUAUCGGAGUUG |
| hsa-miR-200a | 0.39 | 0.000164 | 1p | 36 | UAACACUGUCUGGUAACGAUGU |

* Only the top 35 significantly dysregulated miRNAs are shown here. For a complete list of all 166 dysregulated miRNAs, see Table 12.

TABLE 10
A partial list of the differentially expressed miRNAs in clear-cell RCC and their
association with other types of cancer.

| miRNA | Cancer | Function | Target | Reference |
|---|---|---|---|---|
| hsa-miR-122 | Downregulated in | Cell cycle and | Bcl-W | (1) |
| | HCC^{a} | apoptosis | CCNG1 | |
| | | | Cyclin G1 | (2) |
| hsa-miR-155 | Increased in breast | Tight junction | RHOA1 | (3) |
| | cancer | dissolution, cell | | |
| | | polarity, EMT | | |
| | | regulation | | |
| | | Cell survival and | FOXO3 | (4) |
| | | chemoresistance | | |
| | | Cell proliferation | SOCS1 | (5) |
| | Increased in B-cell | Cell proliferation | SHIP1 | (6) |
| | lymphona | | | |
| has-miR-21 | Increased in | Involved in | PDCD4 | (7-10) |
| | multiple cancers | cellular growth, | RECK | (11, 12) |
| | | migration, and | TIMP3 | (12) |
| | | invasion | MARKS | (13) |
| | | | LRRF1P1 | (14) |
| | | | PTEN | (15, 16) |
| | | | Sprouty2 | (17) |
| | | | TPM1 | (18) |
| | | | RHOB | (19) |
| hsa-miR-210 | Increased upon | Increased cell | Ephrin-A | (20, 21) |
| | hypoxia in | survival and | | |
| | endothelial cells | migration | | |
| | Increased in | Regulate cell | E2F3 | (22) |
| | ovarian cancer | cycle | | |
| | Increased in | Response to | MNT | (23) |
| | multiple cancers | hypoxia | | |
| hsa-miR-34a | Decreased in | Involved in | E2F3 | (24) |
| | neuroblastoma | tumor cell | | |
| | | proliferation, | MYNC | (25, 26) |
| | | migration and | BCL2 | (26) |
| | Decreased in HCC^{a} | invasion | c-Met | (27) |
| hsa-miR-15a | Decreased in CLL^{b} | Involved in cell | BCL2 | (28) |
| | Decreased in | proliferation, cell | Bmi1 | (29) |
| | ovarian cancer | cycle and | | |
| | Decreased in lung | proliferation | Cyclins D1, D2, E1 | (30) |
| | cancer | | | |
| hsa-miR-15b | Decreased in | Involved in cell | CCNE1 | (31) |
| | glioma | cycle regulation | | |
| | Decreased in | and multidrug | Bcl2 | (32) |
| | gastric cancer | resistance | | |
| hsa-miR-223 | Decreased in AML^{c} | Involved in | E2F1 | (33) |
| | | regulation of the | | |
| | | cell cycle | | |
| hsa-miR-451 | Decreased in | Cell proliferation | MIF | (34) |
| | gastric and colon | | | |
| | cancer | | | |
| | Increased in | Drug resistance | MDR1/P-glycoprotein | (35) |
| | multidrug resistant | | | |
| | cell lines | | | |
| hsa-miR-150 | Increased in gastric | Cell proliferation | EGR2 | (36) |
| | cancer | | | |
| hsa-miR-224 | Increased in HCC^{a} | Apoptotic cell | API-5 | (37) |
| | | death | | |
| hsa-miR-106b | Increased in | Involved in cell | p21/CDKN1 | (38) |
| | multiple tumors | cycle | | |
| | | | E2F1 | (39) |
| hsa-miR-146b | Increased in glioma | Cell migration | MMP16 | (40) |
| | | and invasion | | |
| hsa-miR-130b | Increased in gastric | Involved in the | RNUX3 | (41) |
| | cancer | regulation of | | |
| | | cell viability | | |
| miR-200 family | Decreased in breast | Repression | ZEB1 | (42-46) |
| (hsa-miR-200a, | cancer | during EMT | ZEB2 | |
| hsa-miR-200b, | | sustains the | TGFβ2 | |
| hsa-miR-200c, | | process | | |
| hsa-miR-141, | | | | |
| hsa-miR-429) | | | | |
| hsa-miR-335 | Decreased in breast | Cellular | SOX4 | (47) |
| | cancer | migration and | | |
| | | invasion | | |
| hsa-miR-218 | Decreased in | Cellular | Robo1 receptor | (48) |
| | gastric cancer | migration and | | |
| | | invasion | | |
| hsa-miR-532- | Increased in | Cell progression | RUNX3 | (49) |
| 5p | melanoma | | | |

1. HCC, hepatocellular carcinoma
2. CLL, chronic lymphocytic leukemia
3. AML, acute myeloid leukemia
(1) Ma L, Liu J, Shen J, Liu L, Wu J, Li W, et al. Expression of miR-122 mediated by adenoviral vector induces apoptosis and cell cycle arrest of cancer cells. Cancer Biol Ther 2010; 9.
(2) Gramantieri L, Ferracin M, Fornari F, Veronese A, Sabbioni S, Liu CG, et al. Cyclin G1 is a target of miR-122a, a microRNA frequently down-regulated in human hepatocellular carcinoma. Cancer Res 2007; 67: 6092-9.
(3) Kong W, Yang H, He L, Zhao JJ, Coppola D, Dalton WS, et al. MicroRNA-155 is regulated by the transforming growth factor beta/Smad pathway and contributes to epithelial cell plasticity by targeting RhoA. Mol Cell Biol 2008; 28: 6773-84.
(4) Kong W, He L, Coppola M, Guo J, Esposito NN, Coppola D, et al. MicroRNA-155 regulates cell survival, growth and chemosensitivity by targeting FOXO3a in breast cancer. J Biol Chem 2010.
(5) Jiang S, Zhang HW, Lu MH, He XH, Li Y, Gu H, et al. MicroRNA-155 functions as an OncomiR in breast cancer by targeting the suppressor of cytokine signaling 1 gene. Cancer Res 2010; 70: 3119-27.
(6) Pedersen IM, Otero D, Kao E, Miletic AV, Hother C, Ralfkiaer E, et al. Onco-miR-155 targets SHIP1 to promote TNFalpha-dependent growth of B cell lymphomas. EMBO Mol Med 2009; 1: 288-95.
(7) Lu Z, Liu M, Stribinskis V, Klinge CM, Ramos KS, Colburn NH, et al. MicroRNA-21 promotes cell transformation by targeting the programmed cell death 4 gene. Oncogene 2008; 27: 4373-9.
(8) Asangani IA, Rasheed SA, Nikolova DA, Leupold JH, Colburn NH, Post S, et al. MicroRNA-21 (miR-21) post-transcriptionally downregulates tumor suppressor Pdcd4 and stimulates invasion, intravasation and metastasis in colorectal cancer. Oncogene 2008; 27: 2128-36.
(9) Yao Q, Xu H, Zhang QQ, Zhou H, Qu LH. MicroRNA-21 promotes cell proliferation and down-regulates the expression of programmed cell death 4 (PDCD4) in HeLa cervical carcinoma cells. Biochem Biophys Res Commun 2009; 388: 539-42.
(10) Frankel LB, Christoffersen NR, Jacobsen A, Lindow M, Krogh A, Lund AH. Programmed cell death 4 (PDCD4) is an important functional target of the microRNA miR-21 in breast cancer cells. J Biol Chem 2008; 283: 1026-33.
(11) Gabriely G, Wurdinger T, Kesari S, Esau CC, Burchard J, Linsley PS, et al. MicroRNA 21 promotes glioma invasion by targeting matrix metalloproteinase regulators. Mol Cell Biol 2008; 28: 5369-80.
(12) Song B, Wang C, Liu J, Wang X, Lv L, Wei L, et al. MicroRNA-21 regulates breast cancer invasion partly by targeting tissue inhibitor of metalloproteinase 3 expression. J Exp Clin Cancer Res 2010; 29: 29.
(13) Li T, Li D, Sha J, Sun P, Huang Y. MicroRNA-21 directly targets MARCKS and promotes apoptosis resistance and invasion in prostate cancer cells. Biochem Biophys Res Commun 2009; 383: 280-5.
(14) Li Y, Li W, Yang Y, Lu Y, He C, Hu G, et al. MicroRNA-21 targets LRRFIP1 and contributes to VM-26 resistance in glioblastoma multiforme. Brain Res 2009; 1286: 13-8.
(15) Meng F, Henson R, Wehbe-Janek H, Ghoshal K, Jacob ST, Patel T. MicroRNA-21 regulates expression of the PTEN tumor suppressor gene in human hepatocellular cancer. Gastroenterology 2007; 133: 647-58.
(16) Zhang JG, Wang JJ, Zhao F, Liu Q, Jiang K, Yang GH. MicroRNA-21 (miR-21) represses tumor suppressor PTEN and promotes growth and invasion in non-small cell lung cancer (NSCLC). Clin Chim Acta 2010.
(17) Sayed D, Rane S, Lypowy J, He M, Chen IY, Vashistha H, et al. MicroRNA-21 Targets Sprouty2 and Promotes Cellular Outgrowths. Mol Biol Cell 2008.
(18) Zhu S, Si ML, Wu H, Mo YY. MicroRNA-21 targets the tumor suppressor gene tropomyosin 1 (TPM1). J Biol Chem 2007; 282: 14328-36.
(19) Connolly EC, Van DK, Rogler LE, Rogler CE. Overexpression of miR-21 promotes an in vitro metastatic phenotype by targeting the tumor suppressor RHOB. Mol Cancer Res 2010; 8: 691-700.
(20) Fasanaro P, D'Alessandra Y, Di S, V, Melchionna R, Romani S, Pompilio G, et al. MicroRNA-210 Modulates Endothelial Cell Response to Hypoxia and Inhibits the Receptor Tyrosine Kinase Ligand Ephrin-A3. J Biol Chem 2008; 283: 15878-83.
(21) Pulkkinen K, Maim T, Turunen M, Koistinaho J, Yla-Herttuala S. Hypoxia induces microRNA miR-210 in vitro and in vivo Ephrin-A3 and neuronal pentraxin 1 are potentially regulated by miR-210. FEBS Lett 2008.
(22) Giannakakis A, Sandaltzopoulos R, Greshock J, Liang S, Huang J, Hasegawa K, et al. miR-210 links hypoxia with cell cycle regulation and is deleted in human epithelial ovarian cancer Cancer Biol Ther 2007; 7.
(23) Zhang Z, Sun H, Dai H, Walsh RM, Imakura M, Schelter J, et al. MicroRNA miR-210 modulates cellular response to hypoxia through the MYC antagonist MNT. Cell Cycle 2009; 8: 2756-68.
(24) Welch C, Chen Y, Stallings RL. MicroRNA-34a functions as a potential tumor suppressor by inducing apoptosis in neuroblastoma cells. Oncogene 2007; 26: 5017-22.
(25) Wei JS, Song YK, Durinck S, Chen QR, Cheuk AT, Tsang P, et al. The MYCN oncogene is a direct target of miR-34a. Oncogene 2008; 27: 5204-13.
(26) Cole KA, Attiyeh EF, Mosse YP, Laquaglia MJ, Diskin SJ, Brodeur GM, et al. A functional screen identifies miR-34a as a candidate neuroblastoma tumor suppressor gene. Mol Cancer Res 2008; 6: 735-42.
(27) Li N, Fu H, Tie Y, Hu Z, Kong W, Wu Y, et al. miR-34a inhibits migration and invasion by down-regulation of c-Met expression in human hepatocellular carcinoma cells. Cancer Lett 2009; 275: 44-53.
(28) Cimmino A, Calin GA, Fabbri M, Iorio MV, Ferracin M, Shimizu M, et al. miR-15 and miR-16 induce apoptosis by targeting BCL2. Proc Natl Acad Sci USA 2005; 102: 13944-9.
(29) Bhattacharya R, Nicoloso M, Arvizo R, Wang E, Cortez A, Rossi S, et al. MiR-15a and MiR-16 control Bmi-1 expression in ovarian cancer. Cancer Res 2009; 69: 9090-5.
(30) Bandi N, Zbinden S, Gugger M, Arnold M, Kocher V, Hasan L, et al. miR-15a and miR-16 are implicated in cell cycle regulation in a Rb-dependent manner and are frequently deleted or down-regulated in non-small cell lung cancer. Cancer Res 2009; 69: 5553-9.
(31) Xia H, Qi Y, Ng SS, Chen X, Chen S, Fang M, et al. MicroRNA-15b regulates cell cycle progression by targeting cyclins in glioma cells. Biochem Biophys Res Commun 2009; 380: 205-10.
(32) Xia L, Zhang D, Du R, Pan Y, Zhao L, Sun S, et al. miR-15b and miR-16 modulate multidrug resistance by targeting BCL2 in human gastric cancer cells. Int J Cancer 2008; 123: 372-9.
(33) Pulikkan JA, Dengler V, Peramangalam PS, Peer Zada AA, Muller-Tidow C, Bohlander SK, et al. Cell-cycle regulator E2F1 and microRNA-223 comprise an autoregulatory negative feedback loop in acute myeloid leukemia. Blood 2010; 115: 1768-78.
(34) Bandres E, Bitarte N, Arias F, Agorreta J, Fortes P, Agirre X, et al. microRNA-451 regulates macrophage migration inhibitory factor production and proliferation of gastrointestinal cancer cells. Clin Cancer Res 2009; 15: 2281-90.
(35) Zhu H, Wu H, Liu X, Evans BR, Medina DJ, Liu CG, et al. Role of MicroRNA miR-27a and miR-451 in the regulation of MDR1/P-glycoprotein expression in human cancer cells. Biochem Pharmacol 2008; 76: 582-8.
(36) Wu Q, Jin H, Yang Z, Luo G, Lu Y, Li K, et al. MiR-150 promotes gastric cancer proliferation by negatively regulating the pro-apoptotic gene EGR2. Biochem Biophys Res Commun 2010; 392: 340-5.
(37) Wang Y, Lee AT, Ma JZ, Wang J, Ren J, Yang Y, et al. Profiling microRNA expression in hepatocellular carcinoma reveals microRNA-224 up-regulation and apoptosis inhibitor-5 as a microRNA-224-specific target. J Biol Chem 2008; 283: 13205-15.
(38) lvanovska I, Ball AS, Diaz RL, Magnus JF, Kibukawa M, Schelter JM, et al. MicroRNAs in the miR-106b family regulate p21/CDKN1A and promote cell cycle progression Mol Cell Biol 2008; 28: 2167-74.
(39) Li Y, Tan W, Neo TW, Aung MO, Wasser S, Lim SG, et al. Role of the miR-106b-25 microRNA cluster in hepatocellular carcinoma. Cancer Sci 2009; 100: 1234-42.
(40) Xia H, Qi Y, Ng SS, Chen X, Li D, Chen S, et al. microRNA-146b inhibits glioma cell migration and invasion by targeting MMPs. Brain Res 2009; 1269: 158-65.
(41) Lai KW, Koh KX, Loh M, Tada K, Subramaniam MM, Lim XY, et al. MicroRNA-130b regulates the tumour suppressor RUNX3 in gastric cancer. Eur J Cancer 2010; 46: 1456-63.
(42) Gregory PA, Bert AG, Paterson EL, Barry SC, Tsykin A, Farshid G, et al. The miR-200 family and miR-205 regulate epithelial to mesenchymal transition by targeting ZEB1 and SIP1. Nat Cell Biol 2008; 10: 593-601.
(43) Korpal M, Lee ES, Hu G, Kang Y. The miR-200 family inhibits epithelial-mesenchymal transition and cancer cell migration by direct targeting of E-cadherin transcriptional repressors ZEB1 and ZEB2. J Biol Chem 2008; 283: 14910-4.
(44) Bracken CP, Gregory PA, Kolesnikoff N, Bert AG, Wang J, Shannon MF, et al. A double-negative feedback loop between ZEB1-SIP1 and the microRNA-200 family regulates epithelial-mesenchymal transition. Cancer Res 2008; 68: 7846-54.
(45) Burk U, Schubert J, Wellner U, Schmalhofer O, Vincan E, Spaderna S, et al. A reciprocal repression between ZEB1 and members of the miR-200 family promotes EMT and invasion in cancer cells. EMBO Rep 2008; 9: 582-9.
(46) Gregory PA, Bracken CP, Bert AG, Goodall GJ. MicroRNAs as regulators of epithelial-mesenchymal transition. Cell Cycle 2008; 7: 3112-8.
(47) Tavazoie SF, Alarcon C, Oskarsson T, Padua D, Wang Q, Bos PD, et al. Endogenous human microRNAs that suppress breast cancer metastasis. Nature 2008; 451: 147-52.
(48) Tie J, Pan Y, Zhao L, Wu K, Liu J, Sun S, et al. MiR-218 inhibits invasion and metastasis of gastric cancer by targeting the Robo1 receptor. PLoS Genet 2010; 6: e1000879.
(49) Kitago M, Martinez SR, Nakamura T, Sim MS, Hoon DS. Regulation of RUNX3 tumor suppressor gene expression in cutaneous melanoma. Clin Cancer Res 2009; 15: 2988-94.

TABLE 11
Hypoxia inducible miRNAs that are dysregulated in clear-cell RCC.

| | | | Dysregulation | |
|---|---|---|---|---|
| miRNA | Fold change | p-value | previously reported | Reference |
| hsa-let-7e | 1.66 | 8.58E−05 | Up | (1) |
| hsa-let-7g | 1.26 | 0.0008547 | Up | (1) |
| hsa-let-7i | 1.64 | 2.60E−06 | Up | (1) |
| hsa-miR- | 1.37 | 0.0146308 | Up | (2) |
| 106a | | | | |
| hsa-miR- | 1.39 | 0.0024526 | Up | (2) |
| 181a | | | | |
| hsa-miR-195 | 1.39 | 0.0192754 | Up | (2) |
| hsa-miR-21 | 3.16 | 5.70E−06 | Up | (2) |
| hsa-miR-210 | 9.47 | <1e−07 | Up | (1, 2) |
| hsa-miR-24 | 1.55 | 7.88E−05 | Up | (2) |
| hsa-miR-30e | 0.33 | 2.12E−05 | Down | (1) |
| hsa-miR-93 | 1.92 | 1.69E−05 | Up | (2) |

(1) Hebert C, Norris K, Scheper MA, Nikitakis N, Sauk JJ. High mobility group A2 is a target for miRNA-98 in head and neck squamous cell carcinoma. Mol Cancer 2007; 6: 5.
(2) Kulshreshtha R, Ferracin M, Wojcik SE, Garzon R, Alder H, Agosto-Perez FJ, et al. A microRNA signature of hypoxia. Mol Cell Biol 2007; 27: 1859-67.

TABLE 12
A complete list of 166 differentially expressed (89 upregulated and 77
downregulated) miRNAs in clear cell renal cell carcinoma when compared to
normal kidney tissue.

| | p- | Fold | Ch. | | Mature | Accession |
|---|---|---|---|---|---|---|
| miRNA ID | value | Change | Arm | Band | Sequence | Number |
| Upregulated in ccRCC | | | | | | |
| hsa-miR-122 | 28.16 | <1e−07 | 18q | 21 | UGGAGUGUGACAAUGGUGUU | MIMAT0000421 |
| | | | | | UG | |
| hsa-miR-155 | 11.24 | 1.00E−07 | 21q | 21 | UUAAUGCUAAUCGUGAUAGG | MIMAT0000646 |
| | | | | | GGU | |
| hsa-miR-210 | 9.47 | <1e−07 | 11p | 15 | CUGUGCGUGUGACAGCGGCU | MIMAT0000267 |
| | | | | | GA | |
| hsa-miR-34a | 4.9 | 3.00E−07 | 1p | 36 | UGGCAGUGUCUUAGCUGGUU | MIMAT0000255 |
| | | | | | GU | |
| hsa-miR-15a | 3.76 | 5.80E−06 | 13q | 14 | UAGCAGCACAUAAUGGUUUGU | MIMAT0000068 |
| | | | | | G | |
| hsa-miR-223 | 3.53 | 0.00017 | Xq | 12 | UGUCAGUUUGUCAAAUACCCC | MIMAT0000280 |
| | | | | | A | |
| hsa-miR-21 | 3.16 | 5.70E−06 | 17q | 23 | UAGCUUAUCAGACUGAUGUU | MIMAT0000076 |
| | | | | | GA | |
| hsa-miR-451 | 3.06 | 0.004842 | 17q | 11 | AAACCGUUACCAUUACUGAGU | MIMAT0001631 |
| | | | | | U | |
| hsa-miR-15b | 3.04 | 6.00E−07 | 3q | 26 | UAGCAGCACAUCAUGGUUUAC | MIMAT0000417 |
| | | | | | A | |
| hsa-miR-342- | 3.03 | 2.45E05 | 14q | 32 | UCUCACACAGAAAUCGCACCC | MIMAT0000753 |
| 3p | | | | | GU | |
| hsa-miR-224 | 3.01 | 8.60E−06 | Xq | 28 | CAAGUCACUAGUGGUUCCGU | MIMAT0000281 |
| | | | | | U | |
| hsa-miR-486- | 2.89 | 0.006009 | 8p | 11 | UCCUGUACUGAGCUGCCCCG | MIMAT0002177 |
| 5p | | | | | AG | |
| hsa-miR-106b | 2.78 | 6.00E−07 | 7q | 22 | UAAAGUGCUGACAGUGCAGAU | MIMAT0000680 |
| hsa-miR-150 | 2.62 | 0.013689 | 19q | 13 | UCUCCCAACCCUUGUACCAGU | MIMAT0000451 |
| | | | | | G | |
| hsa-miR-342- | 2.32 | 1.00E−06 | 14q | 32 | AGGGGUGCUAUCUGUGAUUG | MIMAT0004694 |
| 5p | | | | | A | |
| hsa-miR-146b- | 2.27 | 0.001901 | 10q | 24 | UGAGAACUGAAUUCCAUAGGC | MIMAT0002809 |
| 5p | | | | | U | |
| hsa-miR-130b | 2.22 | <1e−07 | 22q | 11 | CAGUGCAAUGAUGAAAGGGCA | MIMAT0000691 |
| | | | | | U | |
| hsa-miR-1270 | 2.15 | 7.76E05 | 19p | 12 | CUGGAGAUAUGGAAGAGCUG | MIMAT0005924 |
| | | | | | UGU | |
| hsa-miR-489 | 2.14 | 0.03382 | 7q | 21 | GUGACAUCACAUAUACGGCAG | MIMAT0002805 |
| | | | | | C | |
| hsa-miR-146a | 2.04 | 0.002297 | 5q | 34 | UGAGAACUGAAUUCCAUGGG | MIMAT0000449 |
| | | | | | UU | |
| hsa-miR-16 | 2.02 | 1.03E−05 | 13q | 14 | UAGCAGCACGUAAAUAUUGGC | MIMAT0000069 |
| | | | | | G | |
| hsa-miR-28-3p | 2.02 | 1.10E−06 | 3q | 28 | CACUAGAUUGUGAGCUCCUG | MIMAT0004502 |
| | | | | | GA | |
| hsa-miR-185 | 1.96 | 1.27E−05 | 22q | 11 | UGGAGAGAAAGGCAGUUCCU | MIMAT0000455 |
| | | | | | GA | |
| hsa-miR-28-5p | 1.93 | 0.00022 | 3q | 28 | AAGGAGCUCACAGUCUAUUGA | MIMAT0000085 |
| | | | | | G | |
| hsa-miR-93 | 1.92 | 1.69E−05 | 7q | 22 | CAAAGUGCUGUUCGUGCAGG | MIMAT0000093 |
| | | | | | UAG | |
| hsa-miR-197 | 1.87 | 6.76E−05 | 1p | 13 | UUCACCACCUUCUCCACCCAG | MIMAT0000227 |
| | | | | | C | |
| hsa-miR-181d | 1.86 | 0.003279 | 19p | 13 | AACAUUCAUUGUUGUCGGUG | MIMAT0002821 |
| | | | | | GGU | |
| hsa-miR-454 | 1.86 | 0.006036 | 17q | 22 | UAGUGCAAUAUUGCUUAUAGG | MIMAT0003885 |
| | | | | | GU | |
| hsa-miR-455- | 1.86 | 0.000899 | 9q | 32 | GCAGUCCAUGGGCAUAUACAC | MIMAT0004784 |
| 3p | | | | | | |
| hsa-miR-629 | 1.86 | 2.60E−05 | 15q | 23 | UGGGUUUACGUUGGGAGAAC | MIMAT0004810 |
| | | | | | U | |
| hsa-miR-340 | 1.86 | 0.001809 | 5q | 35 | UUAUAAAGCAAUGAGACUGAU | MIMAT0004692 |
| | | | | | U | |
| hsa-miR-320d | 1.86 | 0.000257 | 13q | 14 | AAAAGCUGGGUUGAGAGGA | MIMAT0006764 |
| hsa-miR-25 | 1.86 | 1.20E−06 | 7q | 22 | CAUUGCACUUGUCUCGGUCU | MIMAT0000081 |
| | | | | | GA | |
| hsa-miR-126* | 1.86 | 0.011809 | 9q | 34 | CAUUAUUACUUUUGGUACGC | MIMAT0000444 |
| | | | | | G | |
| hsa-let-7e | 1.86 | 8.58E−05 | 19q | 13 | UGAGGUAGGAGGUUGUAUAG | MIMAT0000066 |
| | | | | | UU | |
| hsa-let-7f | 1.86 | 7.60E−06 | 9q | 22 | UGAGGUAGUAGAUUGUAUAG | MIMAT0000067 |
| | | | | | UU | |
| hsa-let-7i | 1.86 | 2.60E−06 | 12q | 14 | UGAGGUAGUAGUUUGUGCUG | MIMAT0000415 |
| | | | | | UU | |
| hsa-miR-126 | 1.86 | 7.27E−05 | 9q | 34 | UCGUACCGUGAGUAAUAAUGC | MIMAT0000445 |
| | | | | | G | |
| hsa-let-7a | 1.86 | 4.80E−06 | 9q | 22 | UGAGGUAGUAGGUUGUAUAG | MIMAT0000062 |
| | | | | | UU | |
| hsa-miR-145* | 1.86 | 0.007853 | 5q | 32 | GGAUUCCUGGAAAUACUGUU | MIMAT0004601 |
| | | | | | Cu | |
| hsa-let-7d | 1.86 | 8.00E−07 | 9q | 22 | AGAGGUAGUAGGUUGCAUAG | MIMAT0000065 |
| | | | | | UU | |
| hsa-miR-20a | 1.86 | 0.000618 | 13q | 31 | UAAAGUGCUUAUAGUGCAGG | MIMAT0000075 |
| | | | | | UAG | |
| hsa-miR-574- | 1.86 | 0.024169 | 3p | 25 | CACGCUCAUGCACACACCCAC | MIMAT0003239 |
| 3p | | | | | A | |
| hsa-miR-320b | 1.55 | 0.00187 | 1p | 13 | AAAAGCUGGGUUGAGAGGGC | MIMAT0005792 |
| | | | | | AA | |
| hsa-let-7c | 1.54 | 2.50E−06 | 21q | 21 | UGAGGUAGUAGGUUGUAUGG | MIMAT0000064 |
| | | | | | UU | |
| hsa-miR-320c | 1.53 | 0.00114 | 18q | 11 | AAAAGCUGGGUUGAGAGGGU | MIMAT0005793 |
| hsa-miR-505* | 1.51 | 2.53E−05 | Xq | 27 | GGGAGCCAGGAAGUAUUGAU | MIMAT0004776 |
| | | | | | GU | |
| hsa-miR-24-2* | 1.5 | 0.000126 | 19p | 13 | UGCCUACUGAGCUGAAACACA | MIMAT0004497 |
| | | | | | G | |
| hsa-miR-143 | 1.5 | 0.007681 | 5q | 32 | UGAGAUGAAGCACUGUAGCU | MIMAT0000435 |
| | | | | | C | |
| hsa-miR-151- | 1.49 | 5.20E−06 | 8q | 24 | UCGAGGAGCUCACAGUCUAG | MIMAT0004697 |
| 5p | | | | | U | |
| hsa-miR-1979 | 1.48 | 0.019955 | 4q | 32 | CUCCCACUGCUUCACUUGACU | MIMAT0009454 |
| | | | | | A | |
| hsa-miR-320a | 1.48 | 0.002086 | 8p | 21 | AAAAGCUGGGUUGAGAGGGC | MIMAT0000510 |
| | | | | | GA | |
| hsa-miR-423- | 1.44 | 0.010699 | 17q | 11 | UGAGGGGCAGAGAGCGAGAC | MIMAT0004748 |
| 5p | | | | | UUU | |
| hsa-let-7b | 1.43 | 3.19E−05 | 22q | 13 | UGAGGUAGUAGGUUGUGUGG | MIMAT0000063 |
| | | | | | UU | |
| hsa-miR-625 | 1.43 | 0.001354 | 14q | 23 | UGAGUGUGUGUGUGUGAGUG | MIMAT0003294 |
| | | | | | UGU | |
| hsa-miR-503 | 1.41 | 0.001843 | Xq | 26 | UAGCAGCGGGAACAGUUCUG | MIMAT0002874 |
| | | | | | CAG | |
| hsa-miR-17 | 1.4 | 0.008017 | 13q | 31 | CAAAGUGCUUACAGUGCAGG | MIMAT0000070 |
| | | | | | UAG | |
| hsa-miR-181a | 1.39 | 0.002453 | 1q | 32 | AACAUUCAACGCUGUCGGUGA | MIMAT0000256 |
| | | | | | GU | |
| hsa-miR-195 | 1.39 | 0.019275 | 17p | 13 | UAGCAGCACAGAAAUAUUGGC | MIMAT0000461 |
| hsa-miR-151- | 1.37 | 0.00012 | 8q | 24 | CUAGACUGAAGCUCCUUGAG | MIMAT0000757 |
| 3p | | | | | G | |
| hsa-miR-455- | 1.37 | 0.012337 | 9q | 32 | UAUGUGCCUUUGGACUACAU | MIMAT0003150 |
| 5p | | | | | CG | |
| hsa-miR-331- | 1.37 | 0.000476 | 12q | 22 | ACUGCCCCAGGUGCUGCUGG | MIMAT0000760 |
| 3p | | | | | | |
| hsa-miR-106a | 1.37 | 0.014631 | Xq | 26 | AAAAGUGCUUACAGUGCAGGU | MIMAT0000103 |
| | | | | | AG | |
| hsa-miR-484 | 1.36 | 0.000492 | 16p | 13 | UCAGGCUCAGUCCCCUCCCG | MIMAT0002174 |
| | | | | | AU | |
| hsa-miR-486- | 1.36 | 0.00178 | 8p | 11 | CGGGGCAGCUCAGUACAGGA | MIMAT0004762 |
| 3p | | | | | U | |
| hsa-miR-497 | 1.33 | 0.023474 | 17p | 13 | CAGCAGCACACUGUGGUUUG | MIMAT0002820 |
| | | | | | U | |
| hsa-miR-885- | 1.31 | 0.016326 | 3p | 25 | UCCAUUACACUACCCUGCCUC | MIMAT0004947 |
| 5p | | | | | U | |
| hsa-miR-361- | 1.28 | 0.002573 | Xq | 21 | UCCCCCAGGUGUGAUUCUGA | MIMAT0004682 |
| 3p | | | | | UUU | |
| hsa-miR-425 | 1.27 | 0.036619 | 3p | 21 | AAUGACACGAUCACUCCCGUU | MIMAT0003393 |
| | | | | | GA | |
| hsa-miR-7-1* | 1.27 | 0.004588 | 9q | 21 | CAACAAAUCACAGUCUGCCAU | MIMAT0004553 |
| | | | | | A | |
| hsa-let-7g | 1.26 | 0.000855 | 3p | 21 | UGAGGUAGUAGUUUGUACAG | MIMAT0000414 |
| | | | | | UU | |
| hsa-miR-92b | 1.25 | 0.00745 | 1q | 22 | UAUUGCACUCGUCCCGGCCU | MIMAT0003218 |
| | | | | | CC | |
| hsa-miR-1301 | 1.21 | 0.027026 | 2p | 23 | UUGCAGCUGCCUGGGAGUGA | MIMAT0005797 |
| | | | | | CUUC | |
| hsa-miR-1913 | 1.21 | 0.021766 | 6q | 27 | UCUGCCCCCUCCGCUGCUGC | MIMAT0007888 |
| | | | | | CA | |
| hsa-miR-18a | 1.2 | 0.029114 | 13q | 31 | UAAGGUGCAUCUAGUGCAGA | MIMAT0000072 |
| | | | | | UAG | |
| hsa-miR-26b | 1.19 | 0.043238 | 2q | 35 | UUCAAGUAAUUCAGGAUAGGU | MIMAT0000083 |
| hsa-miR-16-2* | 1.19 | 0.025278 | 3q | 25 | CCAAUAUUACUGUGCUGCUUU | MIMAT0004518 |
| | | | | | A | |
| hsa-miR-766 | 1.19 | 0.004078 | Xq | 24 | ACUCCAGCCCCACAGCCUCAG | MIMAT0003888 |
| | | | | | C | |
| hsa-miR-224* | 1.18 | 0.035749 | Xq | 28 | AAAAUGGUGCCCUAGUGACUA | MIMAT0009198 |
| | | | | | CA | |
| hsa-miR-92a | 1.17 | 0.021296 | 13q | 31 | UAUUGCACUUGUCCCGGCCU | MIMAT0000092 |
| | | | | | GU | |
| hsa-miR-181a- | 1.15 | 0.01839 | 9q | 33 | ACCACUGACCGUUGACUGUAC | MIMAT0004558 |
| 2* | | | | | C | |
| hsa-miR-29b-1* | 1.15 | 0.012358 | 7q | 32 | GCUGGUUUCAUAUGGUGGUU | MIMAT0004514 |
| | | | | | UAGA | |
| hsa-miR-361- | 1.15 | 0.015719 | Xq | 21 | UUAUCAGAAUCUCCAGGGGUA | MIMAT0000703 |
| 5p | | | | | C | |
| hsa-miR-7 | 1.15 | 0.025632 | 9q | 21 | UGGAAGACUAGUGAUUUUGU | MIMAT0000252 |
| | | | | | UGU | |
| hsa-miR-584 | 1.15 | 0.01747 | 5q | 32 | UUAUGGUUUGCCUGGGACUG | MIMAT0003249 |
| | | | | | AG | |
| hsa-miR-193a- | 1.11 | 0.036752 | 17q | 11 | AACUGGCCUACAAAGUCCCAG | MIMAT0000459 |
| 3p | | | | | U | |
| hsa-miR-139- | 1.02 | 0.044238 | 11q | 13 | GGAGACGCGGCCCUGUUGGA | MIMAT0004552 |
| 3p | | | | | GU | |
| hsa-miR-193a- | 1.6 | 0.000424 | 17q | 11 | UGGGUCUUUGCGGGCGAGAU | MIMAT0004614 |
| 5p | | | | | GA | |
| hsa-miR-24 | 1.55 | 7.88E−05 | 9q | 22 | UGGCUCAGUUCAGCAGGAAC | MIMAT0000080 |
| | | | | | AG | |
| Downregulated in ccRCC | | | | | | |
| hsa-miR-200c | 0.05 | <1e−07 | 12p | 13 | UAAUACUGCCGGGUAAUGAU | MIMAT0000617 |
| | | | | | GGA | |
| hsa-miR-335 | 0.16 | 5.00E−07 | 7q | 32 | UCAAGAGCAAUAACGAAAAAU | MIMAT0000765 |
| | | | | | GU | |
| hsa-miR-218 | 0.18 | 1.40E−06 | 4p | 15 | UUGUGCUUGAUCUAACCAUG | MIMAT0000275 |
| | | | | | U | |
| hsa-miR-1974 | 0.18 | 3.00E−07 | 5q | 15 | UGGUUGUAGUCCGUGCGAGA | MI0009984 |
| | | | | | AUA | |
| hsa-miR-1246 | 0.22 | 0.000485 | 2q | 31 | AAUGGAUUUUUGGAGCAGG | MIMAT0005898 |
| hsa-miR-1977 | 0.23 | 2.00E−07 | 1p | 36 | GAUUAGGGUGCUUAGCUGUU | MI0009987 |
| | | | | | AA | |
| hsa-miR-200b | 0.24 | 2.20E−06 | 1p | 36 | UAAUACUGCCUGGUAAUGAUG | MIMAT0000318 |
| | | | | | A | |
| hsa-miR-429 | 0.26 | 2.00E−07 | 1p | 36 | UAAUACUGUCUGGUAAAACCG | MIMAT0001536 |
| | | | | | U | |
| hsa-miR-187 | 0.28 | 5.70E−06 | 18q | 12 | UCGUGUCUUGUGUUGCAGCC | MIMAT0000262 |
| | | | | | GG | |
| hsa-miR-532- | 0.29 | <1e−07 | Xp | 11 | CAUGCCUUGAGUGUAGGACC | MIMAT0002888 |
| 5p | | | | | GU | |
| hsa-miR-204 | 0.30 | 0.001959 | 9q | 21 | UUCCCUUUGUCAUCCUAUGC | MIMAT0000265 |
| | | | | | CU | |
| hsa-miR-30e | 0.33 | 2.12E−05 | 1p | 34 | UGUAAACAUCCUUGACUGGAA | MIMAT0000692 |
| | | | | | G | |
| hsa-miR-363 | 0.34 | 8.00E−07 | Xq | 26 | AAUUGCACGGUAUCCAUCUGU | MIMAT0000707 |
| | | | | | A | |
| hsa-miR-10a | 0.34 | 4.98E−05 | 17q | 21 | UACCCUGUAGAUCCGAAUUUG | MIMAT0000253 |
| | | | | | UG | |
| hsa-miR-532- | 0.35 | 4.99E−05 | Xp | 11 | CCUCCCACACCCAAGGCUUGC | MIMAT0004780 |
| 3p | | | | | A | |
| hsa-miR-660 | 0.36 | 2.00E−07 | Xp | 11 | UACCCAUUGCAUAUCGGAGUU | MIMAT0003338 |
| | | | | | G | |
| hsa-miR-200a | 0.39 | 0.000164 | 1p | 36 | UAACACUGUCUGGUAACGAUG | MIMAT0000682 |
| | | | | | U | |
| hsa-miR-99a | 0.4 | 1.12E−05 | 21q | 21 | AACCCGUAGAUCCGAUCUUGU | MIMAT0000097 |
| | | | | | G | |
| hsa-miR-502- | 0.4 | 5.50E−06 | Xp | 11 | AAUGCACCUGGGCAAGGAUU | MIMAT0004775 |
| 3p | | | | | CA | |
| hsa-miR-199a- | 0.41 | 6.08E−05 | 19p | 13 | CCCAGUGUUCAGACUACCUG | MIMAT0000231 |
| 5p | | | | | UUC | |
| hsa-miR-500* | 0.42 | 3.70E−06 | Xp | 11 | UAAUCCUUGCUACCUGGGUG | MIMAT0002871 |
| | | | | | AGA | |
| hsa-miR-720 | 0.43 | 0.002713 | 3q | 26 | UCUCGCUGGGGCCUCCA | MIMAT0005954 |
| hsa-miR-10b* | 0.43 | 2.20E−06 | 2q | 31 | ACAGAUUCGAUUCUAGGGGAA | MIMAT0004556 |
| | | | | | U | |
| hsa-miR-194 | 0.44 | 0.00239 | 1q | 41 | UGUAACAGCAACUCCAUGUGG | MIMAT0000460 |
| | | | | | A | |
| hsa-miR-10a* | 0.46 | 3.43E−05 | 17q | 21 | CAAAUUCGUAUCUAGGGGAAU | MIMAT0004555 |
| | | | | | A | |
| hsa-miR-1915 | 0.48 | 0.007719 | 10p | 12 | CCCCAGGGCGACGCGGCGGG | MIMAT0007892 |
| hsa-miR-362- | 0.48 | 1.86E−05 | Xp | 11 | AAUCCUUGGAACCUAGGUGU | MIMAT0000705 |
| 5p | | | | | GAGU | |
| hsa-miR-30a | 0.48 | 9.10E−06 | 6q | 13 | UGUAAACAUCCUCGACUGGAA | MIMAT0000087 |
| | | | | | G | |
| hsa-miR-30e* | 0.49 | 0.008022 | 1p | 34 | CUUUCAGUCGGAUGUUUACA | MIMAT0000693 |
| | | | | | GC | |
| hsa-miR-199a- | 0.49 | 0.003563 | 19p | 13 | ACAGUAGUCUGCACAUUGGU | MIMAT0000232 |
| 3p | | | | | UA | |
| hsa-miR-30a* | 0.49 | 0.002141 | 6q | 13 | UGUAAACAUCCUCGACUGGAA | MIMAT0000088 |
| | | | | | G | |
| hsa-miR-30d | 0.5 | 2.00E−07 | 8q | 24 | UGUAAACAUCCCCGACUGGAA | MIMAT0000245 |
| | | | | | G | |
| hsa-miR-215 | 0.5 | 0.028542 | 1q | 41 | AUGACCUAUGAAUUGACAGAC | MIMAT0000272 |
| hsa-miR-100 | 0.5 | 0.000571 | 11q | 24 | AACCCGUAGAUCCGAACUUGU | MIMAT0000098 |
| | | | | | G | |
| hsa-miR-184 | 0.52 | 0.000188 | 15q | 25 | UGGACGGAGAACUGAUAAGG | MIMAT0000454 |
| | | | | | GU | |
| hsa-miR-494 | 0.53 | 0.008632 | 14q | 32 | UGAAACAUACACGGGAAACCU | MIMAT0002816 |
| | | | | | C | |
| hsa-miR-509- | 0.53 | 8.30E−06 | Xq | 27 | UGAUUGGUACGUCUGUGGGU | MIMAT0002881 |
| 3p | | | | | AG | |
| hsa-miR-149* | 0.53 | 0.020616 | 2q | 37 | AGGGAGGGACGGGGGCUGUG | MIMAT0004609 |
| | | | | | C | |
| hsa-miR-203 | 0.54 | 0.048521 | 14q | 32 | GUGAAAUGUUUAGGACCACUA | MIMAT0000264 |
| | | | | | G | |
| hsa-miR-1975 | 0.55 | 0.001068 | 7q | 36 | CCCCCACAACCGCGCUUGACU | MIMAT0009450 |
| | | | | | AGCU | |
| hsa-miR-1826 | 0.55 | 0.000486 | 16p | 11 | AUUGAUCAUCGACACUUCGAA | MIMAT0006766 |
| | | | | | CGCAAU | |
| hsa-miR-378 | 0.55 | 0.015903 | 5q | 32 | ACUGGACUUGGAGUCAGAAG | MIMAT0000732 |
| | | | | | G | |
| hsa-miR-10b | 0.56 | 6.86E−05 | 2q | 31 | UACCCUGUAGAACCGAAUUUG | MIMAT0000254 |
| | | | | | UG | |
| hsa-miR-138 | 0.56 | 0.000241 | 3p | 21 | AGCUGGUGUUGUGAAUCAGG | MIMAT0000430 |
| | | | | | CCG | |
| hsa-miR-127- | 0.57 | 0.000119 | 14q | 32 | UCGGAUCCGUCUGAGCUUGG | MIMAT0000446 |
| 3p | | | | | Cu | |
| hsa-miR-422a | 0.59 | 0.000808 | 15q | 22 | ACUGGACUUAGGGUCAGAAG | MIMAT0001339 |
| | | | | | GC | |
| hsa-miR-1207- | 0.59 | 0.016775 | 8q | 24 | UGGCAGGGAGGCUGGGAGGG | MIMAT0005871 |
| 5p | | | | | G | |
| hsa-miR-125b | 0.59 | 0.0007 | 11q | 24 | UCCCUGAGACCCUAACUUGU | MIMAT0000423 |
| | | | | | GA | |
| hsa-miR-324- | 0.6 | 3.80E−05 | 17p | 13 | CGCAUCCCCUAGGGCAUUGG | MIMAT0000761 |
| 5p | | | | | UGU | |
| hsa-miR-1290 | 0.6 | 0.055302 | 1p | 36 | UGGAUUUUUGGAUCAGGGA | MIMAT0005880 |
| hsa-miR-30c | 0.61 | 0.004302 | 1p | 34 | UGUAAACAUCCUACACUCUCA | MIMAT0000244 |
| | | | | | GC | |
| hsa-miR-1275 | 0.62 | 0.03108 | 6p | 21 | GUGGGGGAGAGGCUGUC | MIMAT0005929 |
| hsa-miR-933 | 0.62 | 0.01505 | 2q | 31 | UGUGCGCAGGGAGACCUCUC | MIMAT0004976 |
| | | | | | CC | |
| hsa-miR-182 | 0.63 | 0.037264 | 7q | 32 | UUUGGCAAUGGUAGAACUCAC | MIMAT0000259 |
| | | | | | ACU | |
| hsa-miR-1307 | 0.63 | 0.009403 | 10q | 24 | ACUCGGCGUGGCGUCGGUCG | MIMAT0005951 |
| | | | | | UG | |
| hsa-miR-214 | 0.63 | 0.011858 | 1q | 24 | ACAGCAGGCACAGACAGGCAG | MIMAT0000271 |
| | | | | | U | |
| hsa-miR-30c-2* | 0.64 | 0.00347 | 6q | 13 | CUGGGAGAAGGCUGUUUACU | MIMAT0004550 |
| | | | | | CU | |
| hsa-miR-183 | 0.65 | 0.014407 | 7q | 32 | UAUGGCACUGGUAGAAUUCAC | MIMAT0000261 |
| | | | | | U | |
| hsa-miR-29c | 0.65 | 0.043709 | 1q | 32 | UAGCACCAUUUGAAAUCGGUU | MIMAT0000681 |
| | | | | | A | |
| hsa-miR-501- | 0.66 | 5.47E−05 | Xp | 11 | AAUCCUUUGUCCCUGGGUGA | MIMAT0002872 |
| 5p | | | | | GA | |
| hsa-miR-139- | 0.67 | 0.032629 | 11q | 13 | UCUACAGUGCACGUGUCUCC | MIMAT0000250 |
| 5p | | | | | AG | |
| hsa-miR-1231 | 0.67 | 0.016951 | 1p | 32 | GUGUCUGGGCGGACAGCUGC | MIMAT0005586 |
| hsa-miR-30b | 0.68 | 0.038334 | 8q | 24 | UGUAAACAUCCUACACUCAGC | MIMAT0000420 |
| | | | | | U | |
| hsa-miR-638 | 0.69 | 0.050114 | 19p | 13 | AGGGAUCGCGGGCGGGUGGC | MIMAT0003308 |
| | | | | | GGCCU | |
| hsa-miR-145 | 0.73 | 0.038204 | 5q | 32 | GUCCAGUUUUCCCAGGAAUC | MIMAT0000437 |
| | | | | | CCU | |
| hsa-miR-125a- | 0.73 | 0.050443 | 19q | 13 | UCCCUGAGACCCUUUAACCUG | MIMAT0000443 |
| 5p | | | | | UGA | |
| hsa-miR-29c* | 0.74 | 0.000869 | 1q | 32 | UGACCGAUUUCUCCUGGUGU | MIMAT0004673 |
| | | | | | UC | |
| hsa-miR-221 | 0.75 | 0.033687 | Xp | 11 | AGCUACAUUGUCUGCUGGGU | MIMAT0000278 |
| | | | | | UUC | |
| hsa-miR-362- | 0.77 | 0.004763 | Xp | 11 | AACACACCUAUUCAAGGAUUC | MIMAT0004683 |
| 3p | | | | | A | |
| hsa-miR-135b* | 0.77 | 0.016227 | 1q | 32 | AUGUAGGGCUAAAAGCCAUG | MIMAT0004698 |
| | | | | | GG | |
| hsa-miR-652 | 0.79 | 0.008486 | Xp | 23 | AAUGGCGCCACUAGGGUUGU | MIMAT0003322 |
| | | | | | G | |
| hsa-miR-487b | 0.81 | 0.032506 | 14q | 32 | AAUCGUACAGGGUCAUCCACU | MIMAT0003180 |
| | | | | | U | |
| hsa-miR-99b | 0.82 | 0.014186 | 19q | 13 | CACCCGUAGAACCGACCUUGC | MIMAT0000689 |
| | | | | | G | |
| hsa-miR-134 | 0.82 | 0.024889 | 14q | 32 | UGUGACUGGUUGACCAGAGG | MIMAT0000447 |
| | | | | | GG | |
| hsa-miR-1201 | 0.84 | 0.037614 | 14q | 11 | AGCCUGAUUAAACACAUGCUC | MIMAT0005864 |
| | | | | | UGA | |
| hsa-miR-500 | 0.85 | 0.044186 | Xp | 11 | UAAUCCUUGCUACCUGGGUG | MIMAT0004773 |
| | | | | | AGA | |
| hsa-miR-216a | 0.94 | 0.054713 | 2p | 16 | UAAUCUCAGCUGGCAACUGU | MIMAT0000273 |
| | | | | | GA | |

What is claimed is:

1. A method of screening, diagnosing or monitoring the progression of renal cancer in a subject, the method comprising:
(a) obtaining a biological sample from the subject;
(b) detecting in the sample an amount of at least one renal cancer marker chosen from the group consisting of:
(i) at least one of the renal cancer markers listed in Table 8;
(ii) at least one polynucleotide encoding at least one of the renal cancer markers listed in Table 8; or
(iii) at least one of the renal cancer markers listed in Table 12 or a precursor thereof; and,
(c) comparing the detected amount of step (b) with a predetermined standard amount, wherein a difference between the detected level of the at least one renal cancer marker and the standard amount is indicative of renal cancer or the progression of renal cancer.

2. The method of claim 1, wherein the renal cancer marker is a protein, miRNA or mRNA.

3. The method of claim 1, wherein the step of detecting the renal cancer marker comprises contacting the sample with at least one binding agent that specifically binds to the renal cancer marker.

4. The method of claim 3, wherein the binding agent is an antibody.

5. The method of claim 1, wherein an amount of the renal cancer marker or polynucleotide significantly higher than the standard amount is indicative of renal cancer.

6. The method of claim 1, wherein an amount of the renal cancer marker or polynucleotide significantly lower than the standard amount is indicative of renal cancer.

7. The method of claim 1, wherein the sample is obtained from a tissue, extract, cell culture, cell lysate, lavage fluid, or physiological fluid of the subject or from renal tumour tissue of the subject.

8. The method of claim 1, wherein the renal cancer marker is a polynucleotide, a Table 12 marker, or a precursor thereof and wherein the marker is detected with at least one oligonucleotide probe that hybridizes to the marker or to complement thereof.

9. The method of claim 8, wherein the renal cancer marker is detected by:
isolating RNA from the sample;
combining the RNA with at least one reagent, to convert the RNA to cDNA;
treating the cDNA with at least one amplification reaction reagent and at least one primer that hybridizes to the cDNA, to produce at least one amplification product;
analyzing the at least one amplification product to detect an amount of RNA encoding the at least one renal cancer marker; and
comparing the amount of RNA to an amount detected against a panel of expected values for normal tissue derived using similar primers.

10. The method of claim 1, wherein the standard is an amount of the renal cancer marker obtained at an earlier time and wherein progression of renal cancer in the subject is monitored by comparing the amount of the renal cancer marker with the standard amount.

11. The method of claim 1, wherein the renal cancer marker is a renal cell carcinoma (RCC) marker or a clear-cell renal cell carcinoma (ccRCC) marker.

12. The method of claim 1, wherein the standard amount comprises a value corresponding to normal levels of the renal cancer marker in a control sample and wherein a significant difference between the amount of the marker detected in the sample and the standard amount is indicative of an aggressive or indolence of the cancer.

13. A method for determining in a subject whether renal cancer has metastasized or is likely to metastasize in the future, comprising comparing:
(a) levels of a renal cancer marker in a sample obtained from the subject, said renal cancer marker comprising:
(i) at least one of the renal cancer markers listed in Table 8,
(ii) at least one polynucleotide encoding at least one of the renal cancer markers listed in Table 8; or
(iii) at least one of the renal cancer markers listed in Table 12 or a precursor thereof;
and
(b) normal levels or non-metastatic levels of the renal cancer marker in a control sample, wherein a significant difference between the levels in the sample and the normal levels or non-metastatic levels is indicative of metastasis of the cancer.

14. A diagnostic composition comprising:
an agent that:
(i) binds to at least one of the renal cancer markers listed in Table 8,
(ii) hybridizes to at least one polynucleotide encoding at least one of the renal cancer markers listed in Table 8, or
(iii) binds to at least one of the renal cancer markers listed in Table 12 or a precursor thereof; and,
a carrier or diluent.

15. A method for assessing the potential efficacy of a test agent for inhibiting renal cancer in a subject, comprising comparing:
(a) levels of a renal cancer marker in a first sample obtained from the subject, said renal cancer marker comprising:
(i) at least one of the renal cancer markers listed in Table 8;
(ii) at least one polynucleotide encoding at least one of the renal cancer markers listed in Table 8; or
(iii) at least one of the renal cancer markers listed in Table 12 or a precursor thereof;
and
(b) levels of the renal cancer marker in a second sample obtained from the subject, said renal cancer marker;
wherein the first sample is exposed to the test agent and the second sample is not exposed to the test agent and wherein a lower level of the renal cancer marker in the first sample relative to the second sample is an indication that the test agent inhibits renal cancer in the subject.

16. An in vivo method for imaging a renal disease, comprising:
(a) injecting a subject with one or more agents that binds to a renal cancer marker listed in Table 8, the agent carrying a label for imaging the renal cancer marker;
(b) allowing the agent to incubate in vivo and to bind with the renal cancer marker; and
(c) detecting the presence of the label localized to diseased kidney tissue.

17. The method of claim 16, wherein the agent is an antibody that specifically bind with the renal cancer marker.

18. A kit for determining the presence of renal cancer in a subject, comprising at least one binding agent that binds to a renal cancer marker comprising:
(i) at least one of the renal cancer markers listed in Table 8, or a part thereof;
(ii) at least one polynucleotide encoding at least one of the renal cancer markers listed in Table 8, or a part thereof; or,
(iii) at least one of the renal cancer markers listed in Table 12 or a precursor thereof, or a part thereof;
wherein the binding agent comprises a detectable substance or an agent that binds directly or indirectly to a detectable substance.

19. The kit as claimed in claim 18, wherein the binding agent comprises an oligonucleotide that hybridizes to (a) a polynucleotide encoding a renal cancer marker listed in Table 8, or (b) a miRNA renal cancer marker listed in Table 12 or a precursor thereof.
